(19) United States
(12) Patent Application Publication
(10) Pub. No.: US 2016/0024540 A1
(43) Pub. Date: Jan. 28, 2016

(54) Process and Intermediates for the Preparation of Pregabalin

(71) Applicant: PFIZER IRELAND PHARMACEUTICALS, County Cork (IE)

(72) Inventors: Sebastien Debarge, Singapore (SG); David Thomas Erdman, Kalamazoo, MI (US); Michael John Karmilowicz, Groton, CT (US); Rajesh Kumar, Groton, CT (US); Padraig Mary O'Neill, Ringaskiddy (IE)

(21) Appl. No.: 14/775,979

(22) PCT Filed: Mar. 25, 2014
(86) PCT No.: PCT/IB2014/060140

§ 371 Date: Sep. 14, 2015

Related U.S. Application Data
(60) Provisional application No. 61/805,786, filed on Mar. 27, 2013.

(51) Int. Cl. C12P 13/00; C12N 9/10; C07C 59/74; C07D 307/60; C07C 227/12; C12P 17/04; C07D 307/66

(52) U.S. Cl. CPC C12P 13/001; C12P 17/04; C12N 9/1096; C07D 307/66; C07D 307/60; C07C 227/12; C07C 59/74

(57) ABSTRACT

The invention provides processes for the manufacture of a compound of formula (I^{A}). The invention further provides improved methods for the conversion of the compound of formula (I^{A}) into pregabalin.

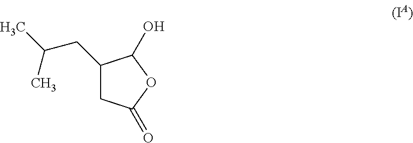

### FIELD OF THE INVENTION

The present invention relates to the manufacture of 5-hydroxy-4-(2-methylpropyl)-3,4-dihydro-5H-2-furanone, and derivatives thereof, for use in the manufacture of (S)-(+)-3-aminomethyl-5-methylhexanoic acid (Pregabalin). The invention further relates to an improved process for the conversion of 5-hydroxy-4-(2-methylpropyl)-3,4-dihydro-5H-2-furanone into Pregabalin. Pregabalin is a γ-amino acid that exhibits binding affinity to the human α₂δ calcium channel subunit.

### BACKGROUND TO THE INVENTION

Pregabalin, or (S)-(+)-3-aminomethyl-5-methyl-hexanoic acid ((S)-II),

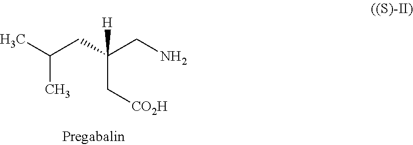

is the active agent in Lyrica®, which is approved for the treatment of epilepsy, neuropathic pain, fibromyalgia and generalized anxiety disorder. It exhibits anti-seizure activity, as discussed in U.S. Pat. No. 5,563,175, and anti-nociceptive activity, as discussed in U.S. Pat. No. 6,001,876. It is hypothesised that the pharmacological activity of Pregabalin (II) is the result of binding to the alpha-2-delta (α₂δ) subunit of a calcium channel. Pregabalin (II) is also described as having utility in other conditions, such as physiological conditions associated with psychomotor stimulants, inflammation, gastrointestinal damage, alcoholism, insomnia, and various psychiatric disorders, including anxiety, depression, mania, and bipolar disorder.

A number of methods for manufacturing Pregabalin have been disclosed. 5-Hydroxy-4-(2-methylpropyl)-3,4-dihydro-5H-2-furanone (I^{A}) has been identified as a useful precursor.

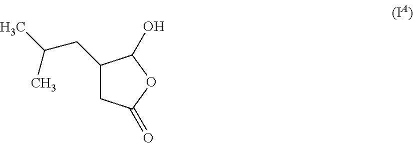

It will be appreciated that compound (I^{A}), in common with a number of the other compounds discussed herein, can be considered to be the cyclized isomer of a 4-oxobutanoic acid derivative. Derivatives of 4-oxobutanoic acid can exist as either the open-chain form, or as the cyclic form.

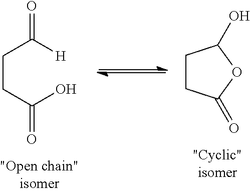 [SMILES: O=CCCC(=O)O ; O=C1CCC(O)O1]

These two isomeric forms may co-exist in equilibrium, and the relative contributions of the two forms will depend on the precise chemical nature of the species.

International Patent Application PCT/US2008/004699 (published as WO2008/127646A2) proposes the conversion of 5-hydroxy-4-(2-methylpropyl)-3,4-dihydro-5H-2-furanone (I^{A}) or an ester of the ring-opened isomeric form (XIII, wherein R^{A} is alkyl) to (II) using a chemical or an enzyme-mediated reductive amination. It is suggested that the use of a transaminase enzyme will provide selectively the preferred ((S)-II) enantiomer.

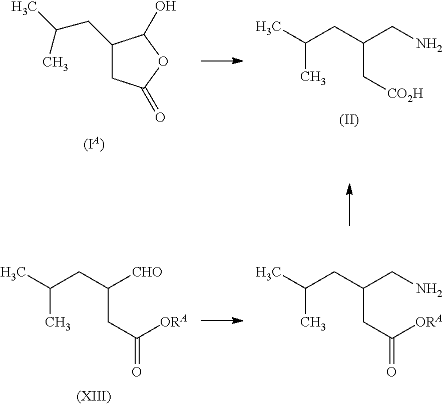

The ester (XIII) is prepared from 4-methylpentanal (III) by alkylation with an appropriate haloacetate in the presence of diisobutylamine. The precursor (I^{A}) is made either by ester hydrolysis of ester (XIII) or by condensation of 4-methylpentanal (III) with glyoxylic acid (IV) and subsequent reduction of the double bond. The use of an enzyme-mediated reduction is also suggested as a way of introducing the desired stereochemistry.

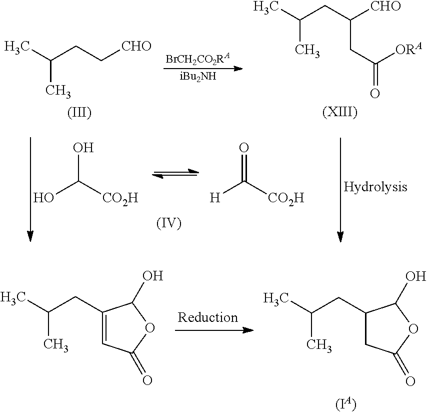

International Patent Application PCT/IN2010/000140 (published as WO2011/086565) discloses a related process. The condensation product of 4-methylpentanal (III) and glyoxylic acid (IV) is reacted with a chiral amine such as α-methylbenzylamine to give a pyrrolone (V). Hydrogenation gives some degree of stereoselectivity, and deprotection gives Pregabalin (II) in chiral form.

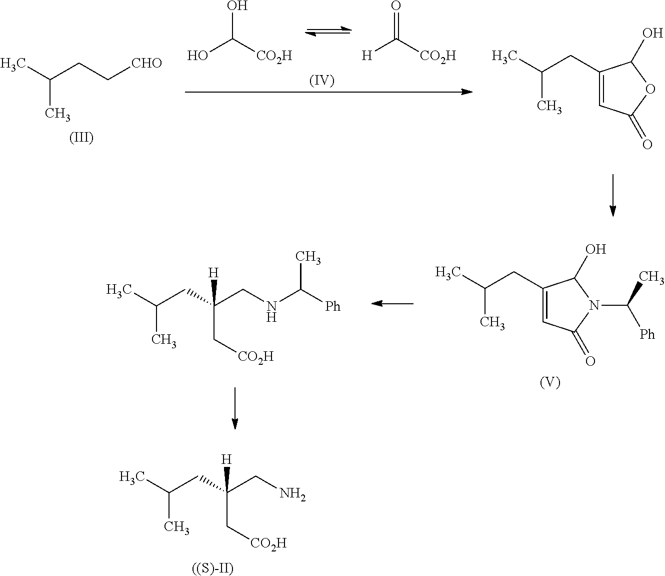

Still further improved syntheses of Pregabalin (II) are sought. It is especially desirable to provide a process which is cost effective and safe. In particular, it is important to provide a synthesis of Pregabalin (II) which can be carried out on a commercial scale, which uses readily available, inexpensive and safe starting materials and reagents, and which avoids the need for difficult separations.

### SUMMARY OF THE INVENTION

The present invention provides improved methods for the manufacture of 5-hydroxy-4-(2-methylpropyl)-3,4-dihydro-5H-2-furanone (I^{A}), and intermediates that are useful in these improved methods.

In a first aspect A1, the invention provides a compound according to formula (VI)

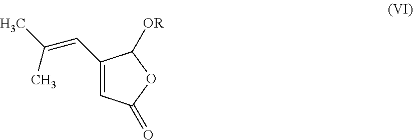

In a first embodiment A1E1, the invention provides a compound according to formula (VI) wherein R is selected from:
hydrogen,
(C₁-C₆)alkyl,
(C₁-C₆)haloalkyl,
(C₁-C₃)alkoxy(C₂-C₆)alkyl,
(C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy,
aryl(C₁-C₆)alkyl, wherein the aryl group may optionally be substituted with 1, 2 or 3 groups independently selected from halo, (C₁-C₃)alkyl, and (C₁-C₃)alkyloxy,
R¹—C(O)—, and

R²—SO₂—;

R¹ is selected from:
hydrogen,
(C₁-C₆)alkyl,
(C₁-C₆)haloalkyl,
(C₁-C₃)alkoxy(C₂-C₆)alkyl,
(C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy, and
aryl(C₁-C₆)alkyl, wherein the aryl group may optionally be substituted with 1, 2 or 3 groups independently selected from halo, (C₁-C₃)alkyl, and (C₁-C₃)alkyloxy,

and R² is selected from:
(C₁-C₆)alkyl,
(C₁-C₆)haloalkyl,
(C₁-C₃)alkoxy(C₂-C₆)alkyl,
(C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy, and
aryl(C₁-C₆)alkyl, wherein the aryl group may optionally be substituted with 1, 2 or 3 groups independently selected from halo, (C₁-C₃)alkyl, and (C₁-C₃)alkyloxy.

In a further embodiment A1E2, the invention provides a compound according to embodiment A1E1 wherein R is hydrogen such that the compound of formula (VI) is 5-hydroxy-4-(2-methyl-1-propenyl)-5H-2-furanone according to formula (VI^{A}).

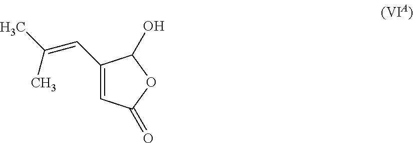

In a further embodiment A1E3, the invention provides a compound according to embodiment A1E1 of formula (VI^{B})

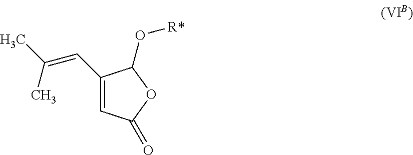

wherein R* is a chiral (C₅-C₁₅) hydrocarbon group.

In a further embodiment A1E4, the invention provides a compound according to embodiment A1E3 wherein R* is selected from (R)- or (S)-α-methylbenzyl, (R)- or (S)-1-(1-naphthyl)ethyl, (R)- or (S)-1-(2-naphthyl)ethyl, menthyl and bornyl, such that the compound of formula (VI) is the compound of formula (VI^{C})-(VI^{K}).

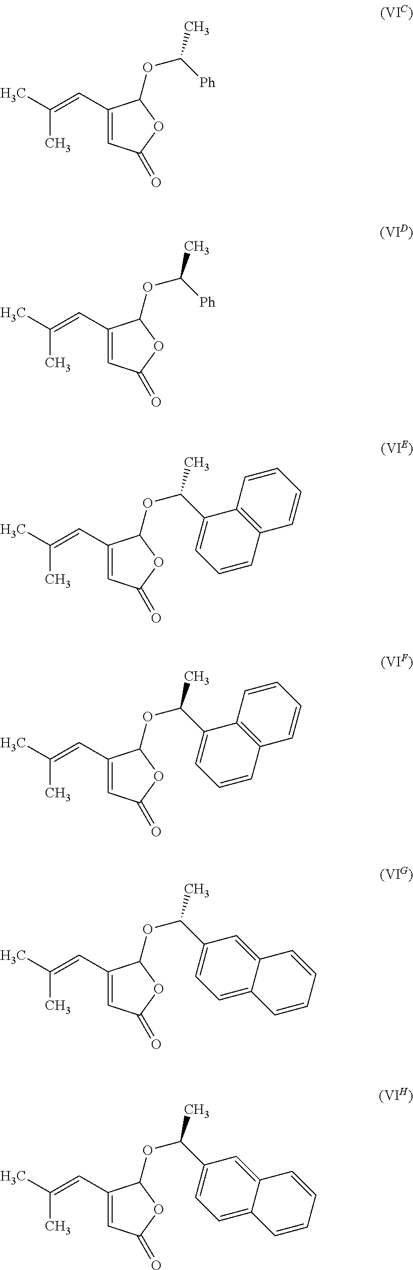 [SMILES: CC(C)=CC1=CC(=O)OC1O[C@H](C)c1ccccc1 ; CC(C)=CC1=CC(=O)OC1O[C@@H](C)c1ccccc1 ; CC(C)=CC1=CC(=O)OC1O[C@H](C)c1cccc2ccccc12 ; CC(C)=CC1=CC(=O)OC1O[C@@H](C)c1cccc2ccccc12 ; CC(C)=CC1=CC(=O)OC1O[C@H](C)c1ccc2ccccc2c1 ; CC(C)=CC1=CC(=O)OC1O[C@@H](C)c1ccc2ccccc2c1]

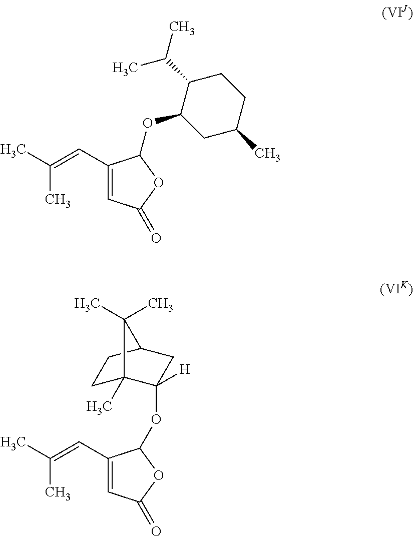

In a further embodiment A1E5, the invention provides a compound according to embodiment A1E1 wherein R is R¹—C(O)— or R²—SO₂— and R¹ and R² are chiral (C₅-C₁₅) hydrocarbon groups.

In another aspect A2, the invention provides a compound of formula (IX)

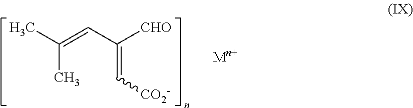

The compound of formula (IX) may exist as either the (E)- or (Z)-geometric isomer, or as a mixture of the two geometric isomers.

In a first embodiment A2E1, the invention provides a compound of formula (IX) wherein:

n is 1 and M⁺ is selected from Li⁺, Na⁺, K⁺, Rb⁺, NH₄⁺, ((C₁-C₃)alkyl)NH₃⁺, ((C₁-C₃)alkyl)₂NH₂⁺, ((C₁-C₃)alkyl)₃NH⁺ and ((C₁-C₃)alkyl)₄N⁺; or
n is 2 and M²⁺ is selected from Mg²⁺, Ca²⁺ and Zn²⁺.

In a further embodiment A2E2, the invention provides a compound according to embodiment A2E1 wherein n is 1 and M⁺ is selected from NH₄⁺ and ((C₁-C₃)alkyl)NH₃⁺.

In a further embodiment A2E3, the invention provides a compound according to embodiment A2E1 wherein n is 1 and M⁺ is selected from Li⁺, Na⁺ and K⁺.

In another aspect A3, the invention provides a compound of formula (VII).

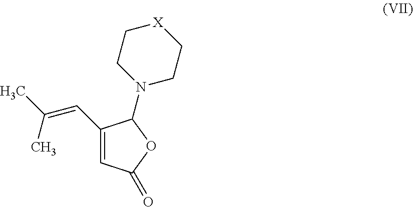

In a first embodiment A3E1, the invention provides a compound of formula (VII) wherein —X— represents a single bond, —CH₂—, —O—; —NH—, —N((C₁-C₃)alkyl)-, —N(benzyl)-, or

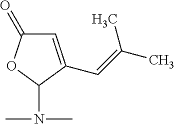

In a further embodiment A3E2, the invention provides a compound according to embodiment A3E1 selected from:
4-(2-methylpropenyl)-5-pyrrolidin-1-yl-5H-furan-2-one;
4-(2-methylpropenyl)-5-piperidin-1-yl-5H-furan-2-one;
4-(2-methylpropenyl)-5-morpholin-4-yl-5H-furan-2-one; and
1,4-bis-(4-(2-methylpropenyl)-5H-furan-2-on-5-yl)piperazine.

In another aspect A4, the invention provides a compound of formula (VIII).

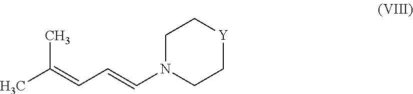

In a first embodiment A4E1, the invention provides a compound of formula (VIII) wherein —Y— represents a single bond, —CH₂—, —O—; —NH—, —N((C₁-C₃)alkyl)-, —N(benzyl)-, or

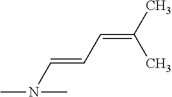

In a further embodiment A4E2, the invention provides a compound according to embodiment A4E1 selected from:
4-(4-methyl-1,3-pentadien-1-yl)morpholine;
1-(4-methyl-1,3-pentadien-1-yl)-piperazine,
1-(4-methyl-1,3-pentadien-1-yl)-4-methylpiperazine,
4-ethyl-1-(4-methyl-1,3-pentadien-1-yl)-piperazine,
4-benzyl-1-(4-methyl-1,3-pentadien-1-yl)-piperazine, and
1,4-bis-(4-methyl-1,3-pentadien-1-yl)piperazine.

In another aspect A5, the invention provides a process for the manufacture of the compound of formula (VI^{A}) comprising the step of treating a compound of formula (VII) with water in the presence of an acid catalyst.

In a first embodiment A5E1, the invention provides a process for the manufacture of the compound of formula (VI^{A}), comprising the steps of:
(a) preparing a compound of formula (VII)

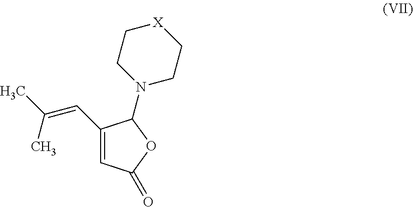

wherein —X— represents a single bond, —CH₂—, —O—, —NH—, —N((C₁-C₃)alkyl)-, —N(benzyl)-, or

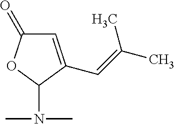

and
(b) treating the compound of formula (VII) with water in the presence of an acid catalyst.

In a further embodiment A5E2, the invention provides a process according to embodiment A5E1 wherein the compound of formula (VII) from step (a) is isolated prior to hydrolysis step (b).

In a further embodiment A5E3, the invention provides a process according to embodiment A5E1 wherein hydrolysis step (b) is carried out directly following step (a) such that the compound of formula (VII) or (VII^{B}) is not isolated prior to hydrolysis step (b).

In a further embodiment A5E4, the invention provides a process according to embodiments A5E1, A5E2 or A5E3 wherein the compound of formula (VII) is prepared by treating a compound of formula (VIII)

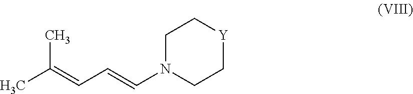

wherein —Y— represents a single bond, —CH₂—, —O—; —NH—, —N((C₁-C₃)alkyl)-, —N(benzyl)-, or

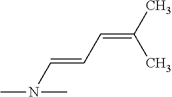

with glyoxylic acid or its hydrate.

In another aspect A6, the invention provides a process for the manufacture of a compound of formula (VI) wherein R is other than hydrogen, R¹—C(O)—, and R²—SO₂—, comprising the step of treating a compound of formula (VI^{A}) with an alcohol R—OH in the presence of an acid catalyst.

In an embodiment A6E1, the invention provides a process for the manufacture of a compound of formula (VI) wherein R is other than hydrogen, R¹—C(O)—, and R²—SO₂—, comprising the steps of:
(a) preparing a compound of formula (VI^{A}) using a process according to any of embodiments A5E1, A5E2, A5E3 and A5E4 as defined above; and
(b) treating the compound of formula (VI^{A}) with an alcohol R—OH in the presence of an acid catalyst.

In another aspect A7, the invention provides a process for the manufacture of a compound of formula (VI) wherein R is other than hydrogen, R¹—C(O)—, and R²—SO₂—, comprising the step of treating a compound of formula (VII) with an alcohol R—OH in the presence of stoichiometric acid.

In an embodiment A7E1, the invention provides a process for the manufacture of a compound of formula (VI) wherein R is other than hydrogen, R¹—C(O)—, and R²—SO₂—, comprising the steps of:
(a) preparing a compound of formula (VII); and
(b) treating the compound of formula (VII) with an alcohol R—OH in the presence of stoichiometric acid.

In another aspect A8, the invention provides a process for the manufacture of a compound of formula (VI) wherein R is R¹—C(O)—, comprising the step of treating a compound of formula (VI^{A}) with an acid chloride R¹—C(O)—Cl or acid anhydride (R¹—C(O))₂O.

In an embodiment A8E1, the invention provides a process for the manufacture of a compound of formula (VI) wherein R is R¹—C(O)—, comprising the steps of:
(a) preparing a compound of formula (VI^{A}) using a process according to any of embodiments A5E1, A5E2, A5E3 and A5E4 as defined above; and
(b) treating the compound of formula (VI^{A}) with an acid chloride R¹—C(O)—Cl or acid anhydride (R¹—C(O))₂O.

In another aspect A9, the invention provides a process for the manufacture of a compound of formula (VI) wherein R is R²—SO₂—, comprising the step of treating a compound of formula (VI^{A}) with a sulfonyl chloride R²—SO₂—Cl.

In an embodiment A9E1, the invention provides a process for the manufacture of a compound of formula (VI) wherein R is R²—SO₂—, comprising the steps of:
(a) preparing a compound of formula (VI^{A}) using a process according to any of embodiments A5E1, A5E2, A5E3 and A5E4 as defined above; and
(b) treating the compound of formula (VI^{A}) with a sulfonyl chloride R²—SO₂—Cl.

In another aspect A10, the invention provides a process for the manufacture of an enamine derivative of 4-methyl-2-pentenal.

In a first embodiment A10E1, the invention provides a process for the manufacture of an enamine derivative of 4-methyl-2-pentenal comprising reacting acetaldehyde with isobutyraldehyde in the presence of a suitable amine.

In a further embodiment A10E2, the invention provides a process according to embodiment A10E1 wherein the suitable amine is a secondary amine.

In a further embodiment A10E3, the invention provides a process according to embodiment A10E2 wherein the secondary amine is selected from: ((C₁-C₄)alkyl)₂NH, pyrrolidine, piperidine, morpholine, piperazine, N-methylpiperazine, N-ethylpiperazine and N-benzylpiperazine.

In a further embodiment A10E4, the invention provides a process according to embodiment A10E3 wherein the secondary amine is selected from pyrrolidine, piperidine, morpholine, and piperazine.

In a further embodiment A10E5, the invention provides a process according to any of embodiments A10E1, A10E2, A10E3 and A10E4 wherein the reaction is performed in the presence of an acid catalyst.

In a further embodiment A10E6, the invention provides a process according to any of embodiments A10E1, A10E2, A10E3, A10E4 and A10E5 wherein the isobutyraldehyde is combined with the suitable amine before addition of the acetaldehyde.

In a further embodiment A10E6, the invention provides a process according to any of embodiments A10E1, A10E2, A10E3, A10E4 and A10E5 wherein the isobutyraldehyde and acetaldehyde are added to the reaction vessel simultaneously.

In another aspect A11, the invention provides a process for the manufacture of 3-aminomethyl-5-methylhexanoic acid (II).

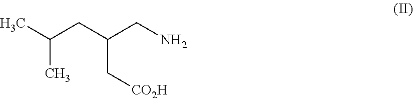

In a first embodiment A11E1, the invention provides a process for the manufacture of 3-aminomethyl-5-methylhexanoic acid (II) or a pharmaceutically acceptable salt thereof, comprising the steps:
(a) preparing 5-hydroxy-4-(2-methyl-1-propenyl)-5H-2-furanone (VI^{A})

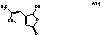

(b) converting said 5-hydroxy-4-(2-methyl-1-propenyl)-5H-2-furanone (VI^{A}) into 5-hydroxy-4-(2-methylpropyl)-3,4-dihydro-5H-2-furanone (I^{A})

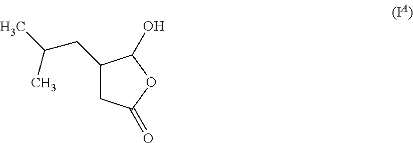

and
(c) converting said 5-hydroxy-4-(2-methylpropyl)-3,4-dihydro-5H-2-furanone (I^{A}) into 3-aminomethyl-5-methylhexanoic acid (II).

In a further embodiment A11E2, the invention provides a process according to embodiment A11E1 wherein the 3-aminomethyl-5-methylhexanoic acid (II) is (S)-3-aminomethyl-5-methylhexanoic acid ((S)-II)

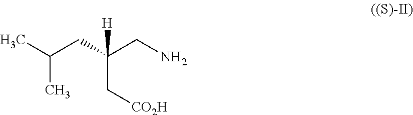

wherein said (S)-3-aminomethyl-5-methylhexanoic acid has an enantiomeric excess of at least 80%.

In a further embodiment A11E3, the invention provides a process according to embodiment A11E1 or A11E2 wherein step (a) comprises a process according to embodiments A5E1, A5E2, A5E3 or A5E4 as described above.

In a further embodiment A11E4, the invention provides a process according to embodiment A11E1, A11E2 or A11E3 wherein step (b) comprises the steps:
(b1) treating the 5-hydroxy-4-(2-methyl-1-propenyl)-5H-2-furanone (VI^{A}) with a metal oxide, hydroxide, carbonate or bicarbonate, ammonia, a mono- di- or tri-(C₁-C₃)alkylamine, or a tetra-(C₁-C₃)alkylammonium hydroxide to form a salt of formula (IX)

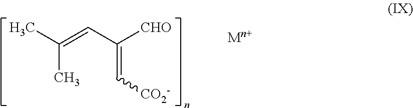

wherein:
n is 1 and M⁺ is selected from Li⁺, Na⁺, K⁺, Rb⁺, NH₄⁺, ((C₁-C₃)alkyl)NH₃⁺, ((C₁-C₃)alkyl)₂NH₂⁺, ((C₁-C₃)alkyl)₃NH⁺ and ((C₁-C₃)alkyl)₄N⁺; or
n is 2 and M²⁺ is selected from Mg²⁺, Ca²⁺ and Zn²⁺;
(b2) hydrogenating the salt of formula (IX) to obtain a salt of formula (X)

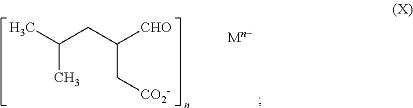

and
(b3) treating the salt of formula (X) with an acid.

In a further embodiment A11E5, the invention provides a process according to embodiment A11E1, A11E2 or A11E3 wherein step (b) comprises the steps:
(b1) converting the 5-hydroxy-4-(2-methyl-1-propenyl)-5H-2-furanone (VI^{A}) to a compound of formula (VI) as defined in embodiment A1E3, wherein R is a chiral (C₅-C₁₅)hydrocarbon group;
(b2) hydrogenating the compound of formula (VI) to obtain a compound of formula (XI)

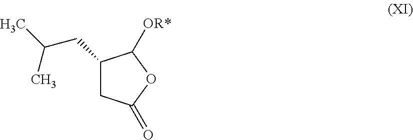

wherein R* is a chiral (C₅-C₁₅)hydrocarbon group; and
(b3) treating the compound of formula (XI) with an acid to give ((S)-I^{A}).

In another aspect A12, the invention provides a further process for the manufacture of 3-aminomethyl-5-methylhexanoic acid (II).

In a first embodiment A12E1, the invention provides a process for the manufacture of 3-aminomethyl-5-methylhexanoic acid (II) or a pharmaceutically acceptable salt thereof, comprising the steps:
(a) preparing 5-hydroxy-4-(2-methyl-1-propenyl)-5H-2-furanone (VI^{A})

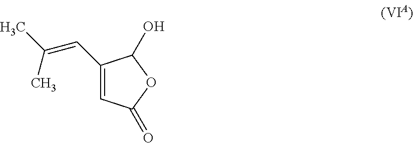

(b) treating the 5-hydroxy-4-(2-methyl-1-propenyl)-5H-2-furanone (VI^{A}) with ammonia or a mono-(C₁-C₃)alkylamine to form a salt of formula (IX^{A})

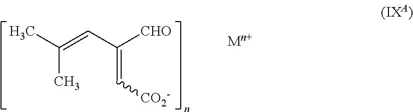

wherein n is 1 and M⁺ is selected from NH₄⁺ and ((C₁-C₃)alkyl)NH₃⁺
(c) hydrogenating the salt of formula (IX^{A}) to obtain a salt of formula (X^{A})

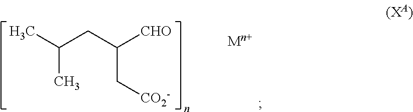

and
(d) treating the salt of formula (X^{A}) with a transaminase or an amine oxidase/imine reductase enzyme to provide 3-aminomethyl-5-methylhexanoic acid (II).

In a further embodiment A12E2, the invention provides a process according to embodiment A12E1 wherein the 3-aminomethyl-5-methylhexanoic acid (II) is (S)-3-aminomethyl-5-methylhexanoic acid ((S)-II)

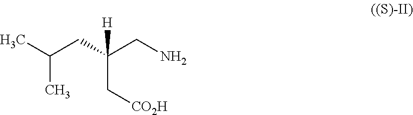

wherein said (S)-3-aminomethyl-5-methylhexanoic acid has an enantiomeric excess of at least 80%.

In a further embodiment A12E3, the invention provides a process according to embodiment A12E1 or A12E2 wherein step (a) comprises a process according to embodiments A5E1, A5E2, A5E3 or A5E4 as described above.

In another aspect A13, the invention provides a further process for the manufacture of 5-hydroxy-4-(2-methylpropyl)-3,4-dihydro-5H-2-furanone (I^{A}).

In a first embodiment A13E1, the invention provides a further process for the manufacture of 5-hydroxy-4-(2-methylpropyl)-3,4-dihydro-5H-2-furanone (I^{A}) which comprises the steps of:
(a) obtaining 3-isobutylidene-2-oxopentanedioic acid (XII^{A}) or its cyclised isomer (XII^{B})

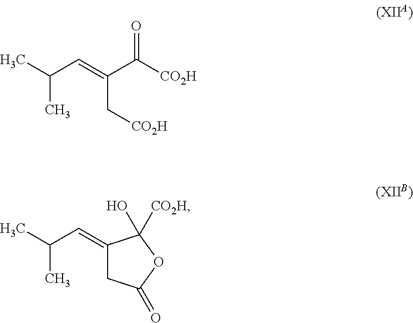

and
(b) sequentially or simultaneously reducing the carbon-carbon double bond and decarboxylating the α-keto acid functional group.

In a further embodiment A13E2, the invention provides a process according to embodiment A13E1 wherein the carbon-carbon double bond is reduced to provide 3-isobutyl-2-oxopentanedioic acid (XV) or its cyclised isomer (XV^{A})

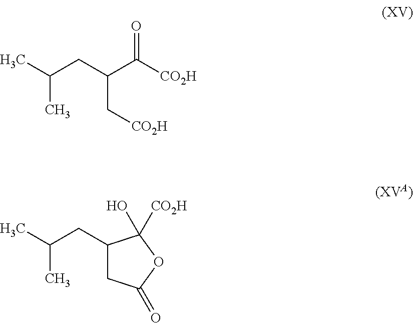

before the decarboxylation of the α-keto acid functional group.

In a further embodiment A13E3, the invention provides a process according to embodiment A13E1 wherein the α-keto acid functional group is decarboxylated to provide 3-formyl-5-methyl-3-pentenoic acid (XVI or its cyclised isomer (XVI^{A})

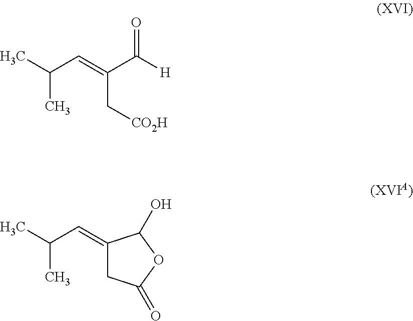

before the reduction of the carbon-carbon double bond.

In a further embodiment A13E4, the invention provides a process according to embodiment A13E1 wherein the α-keto acid functional group is decarboxylated and the carbon-carbon double bond is reduced simultaneously.

In a further embodiment A13E5, the invention provides a process according to embodiments A13E1, A13E2, A13E3 or A13E4 wherein the decarboxylation is carried out in the presence of a decarboxylase enzyme.

In a further embodiment A13E6, the invention provides a process according to embodiments A13E1, A13E2, A13E3, A13E4 or A13E5 wherein the reduction of the carbon-carbon double bond is carried out in the presence of an enoate reductase enzyme.

In another aspect A14, the invention provides a compound selected from:
3-isobutylidene-2-oxopentanedioic acid;
3-isobutyl-2-oxopentanedioic acid; and
3-formyl-5-methyl-3-pentenoic acid,
   or a salt, (C₁-C₆)alkyl ester or cyclised isomer thereof.

In another aspect A15, the invention provides a process for the manufacture of (S)-3-aminomethyl-5-methylhexanoic acid ((S)-II)

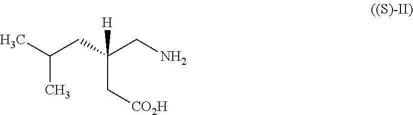

or a pharmaceutically acceptable salt thereof, comprising the steps:
(a) manufacturing 5-hydroxy-4-(2-methylpropyl)-3,4-dihydro-5H-2-furanone (I^{A}) using a process according to any one of embodiments A13E1, A13E2, A13E3, A13E4, A13E5 or A13E6 as defined above, and
(b) converting said 5-hydroxy-4-(2-methylpropyl)-3,4-dihydro-5H-2-furanone into (S)-3-aminomethyl-5-methylhexanoic acid.

In another aspect A16, the invention provides a process for converting (R)-3-aminomethyl-5-methylhexanoic acid into (S)-3-aminomethyl-5-methylhexanoic acid comprising treating the (R)-3-aminomethyl-5-methylhexanoic acid with a transaminase enzyme or an amine oxidase/imine reductase enzyme.

In another aspect A17, the invention provides a process for increasing the proportion of (S)-3-aminomethyl-5-methylhexanoic acid in a mixture of (R)- and (S)-3-aminomethyl-5-methylhexanoic acid comprising treating the mixture with a transaminase enzyme or an amine oxidase/imine reductase enzyme.

In another aspect A18, the invention provides a transaminase enzyme that is useful for the conversion of 5-hydroxy-4-(2-methylpropyl)-3,4-dihydro-5H-2-furanone (I^{A}) into Pregabalin.

In a first embodiment A18E1, the invention provides a transaminase enzyme having an amino acid sequence that has at least 95% homology to the amino acid sequence

(SEQ ID NO. 1)

MNKPQSWEARAETYSLYGFTDMPSLHX²⁷RGTVVVTHGEGPYX⁴¹
VDVX⁴⁵GRRYLDANSGLYNMVAGFDHKGLIDAAKAQYERFPGYH
SFFGRMSDQTVMLSEKLVEVSPFDSGRVFYTNSGSEANDTMVKMLWFLH
AAEGKPQKRKILTRX¹⁴⁷NAYHGVTAVSASMTGX¹⁶³P
X¹⁶⁵NSVFGLPLPGFVHLX¹⁸⁰CPHYVVRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELX³⁰⁴KRLETAIEAIEEFPHGFTAX³²⁴GHPVG
CAIALKAIDVVMNEGLAENVRRLAPRFEERLKHIAERPNIGEYRGIGFM
WALEAVKDKASKTPFDGNLSVSX⁴⁰¹RIANTCX⁴⁰⁸DLGLI
CX⁴¹⁵X⁴¹⁶X⁴¹⁷GQSVILX⁴²⁴PPFILTEAQM
DEMFDKLEKALDKVFAEVA

wherein
X²⁷ is selected from glutamine (Q) and glutamic acid (E);
X⁴¹ is selected from isoleucine (I) and valine (V);
X⁴⁵ is selected from asparigine (N) and histidine (H);
X¹⁴⁷ is selected from asparigine (N) and glutamine (Q);
X¹⁶³ is selected from leucine (L) and methionine (M);
X¹⁶⁵ is selected from tyrosine (Y) and histidine (H);
X¹⁸⁰ is selected from threonine (T); glycine (G) and serine (S);
X³⁰⁴ is selected from alanine (A) and serine (S);
X³²⁴ is selected from glycine (G) and serine (S);
X⁴⁰¹ is selected from lysine (K) and glutamic acid (E);
X⁴⁰⁸ is selected from threonine (T) and glutamine (Q);
X⁴¹⁵ is selected from serine (S) and alanine (A);
X⁴¹⁶ is selected from proline (P) and alanine (A);
X⁴¹⁷ is selected from leucine (L) and methionine (M); and
X⁴²⁴ is selected from cysteine (C) and serine (S).

In a further embodiment A18E2, the invention provides a transaminase enzyme according to embodiment A18E1 having the amino acid sequence of SEQ ID NO. 1.

In a further embodiment A18E3, the invention provides a transaminase enzyme according to embodiment A18E1 or A18E2 wherein:

X²⁷ is glutamic acid (E);
X¹⁴⁷ A is glutamine (Q);
X¹⁶⁵ is histidine (H);
X³⁰⁴ is serine (S);
X³²⁴ is glycine (G);
X⁴⁰¹ is lysine (K);
X⁴⁰⁸ is glutamine (Q);
X⁴¹⁶ is alanine (A);
X⁴¹⁷ is methionine (M); and
X⁴²⁴ is serine (S).

In a further embodiment A18E4, the invention provides a transaminase enzyme according to embodiment A18E2 having an amino acid sequence selected from:

(SEQ ID NO. 2)

MNKPQSWEARAETYSLYGFTDMPSLHQRGTVVVTHGEGPYIVDVNGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRN
NAYHGVTAVSASMTGLPYNSVFGLPLPGFVHLTCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELAKRLETAIEAIEEFPHGFTASGHPVGCAIALKAIDWMNEG
LAENVRRLAPRFEERLKHIAERPNIGEYRGIGFMWALEAVKDKASKTPF
DGNLSVSERIANTCTDLGLICSPMGQSVILCPPFILTEAQMDEMFDKLE
KALDKVFAEVA;

(SEQ ID NO. 3)

MNKPQSWEARAETYSLYGFTDMPSLHERGTVVVTHGEGPYIVDVNGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRN
NAYHGVTAVSASMTGLPYNSVFGLPLPGFVHLTCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELSKRLETAIEAIEEFPHGFTAGGHPVGCAIALKAIDWMNEG
LAENVRRLAPRFEERLKHIAERPNIGEYRGIGFMWALEAVKDKASKTPF
DGNLSVSKRIANTCQDLGLICSALGQSVILCPPFILTEAQMDEMFDKLE
KALDKVFAEVA;

(SEQ ID NO. 4)

MNKPQSWEARAETYSLYGFTDMPSLHERGTVVVTHGEGPYVVDVNGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRQ
NAYHGVTAVSASMTGLPHNSVFGLPLPGFVHLTCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELSKRLETAIEAIEEFPHGFTAGGHPVGCAIALKAIDWMNEG
LAENVRRLAPRFEERLKHIAERPNIGEYRGIGFMWALEAVKDKASKTPF
DGNLSVSKRIANTCQDLGLICSALGQSVILSPPFILTEAQMDEMFDKLE
KALDKVFAEVA;

(SEQ ID NO. 5)

MNKPQSWEARAETYSLYGFTDMPSLHERGTVVVTHGEGPYIVDVNGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRQ
NAYHGVTAVSASMTGMPHNSVFGLPLPGFVHLTCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPALSKRLETAIEAIEEFPHGFTAGGHPVGCAIALKAIDWMNEG
LAENVRRLAPRFEERLKHIAERPNIGEYRGIGFMWALEAVKDKASKTPF
DGNLSVSKRIANTCQDLGLICSAMGQSVILSPPFILTEAQMDEMFDKLE
KALDKVFAEVA;

(SEQ ID NO. 6)

MNKPQSWEARAETYSLYGFTDMPSLHERGTVVVTHGEGPYVVDVNGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRQ
NAYHGVTAVSASMTGLPHNSVFGLPLPGFVHLGCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELSKRLETAIEAIEEFPHGFTAGGHPVGCAIALKAIDWMNEG
LAENVRRLAPRFEERLKHIAERPNIGEYRGIGFMWALEAVKDKASKTPF
DGNLSVSKRIANTCQDLGLICAAMGQSVILSPPFILTEAQMDEMFDKLE
KALDKVFAEVA;
and

(SEQ ID NO. 7)

MNKPQSWEARAETYSLYGFTDMPSLHERGTVVVTHGEGPYIVDVHGRRY
LDANSGLYNMVAGFDHKGLIDAAKAQYERFPGYHSFFGRMSDQTVMLSE
KLVEVSPFDSGRVFYTNSGSEANDTMVKMLWFLHAAEGKPQKRKILTRQ
NAYHGVTAVSASMTGLPHNSVFGLPLPGFVHLSCPHYWRYGEEGETEEQ
FVARLARELEETIQREGADTIAGFFAEPVMGAGGVIPPAKGYFQAILPI
LRKYDIPVISDEVICGFGRTGNTWGCVTYDFTPDAIISSKNLTAGFFPV
GAVILGPELSKRLETAIEAIEEFPHGFTAGGHPVGCAIALKAIDWMNEG
LAENVRRLAPRFEERLKHIAERPNIGEYRGIGFMWALEAVKDKASKTPF
DGNLSVSKRIANTCQDLGLICSAMGQSVILSPPFILTEAQMDEMFDKLE
KALDKVFAEVA.

In another aspect A19, the invention provides a process for the manufacture of (S)-3-aminomethyl-5-methylhexanoic acid ((S)-II)

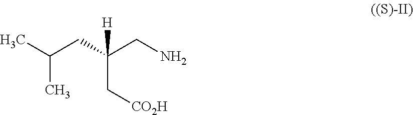

or a pharmaceutically acceptable salt thereof, comprising the step of treating 5-hydroxy-4-(2-methylpropyl)-3,4-dihydro-5H-2-furanone (I^{A}) and an amine with a transaminase enzyme according to any one of embodiments A18E1, A18E2, A18E3 and A18E4.

### DETAILED DESCRIPTION OF PREFERRED EMBODIMENTS

The term “alkyl” means a straight-chain or branched-chain saturated aliphatic hydrocarbon radical containing the specified number of carbon atoms. Examples of alkyl radicals include methyl, ethyl, n-propyl, isopropyl, n-butyl, isobutyl, sec-butyl, tert-butyl, neopentyl and n-hexyl.

The term “alkoxy” means a group made up of an alkyl group as defined above connected to an oxygen atom. Examples of alkoxy groups include methoxy, ethoxy and isopropoxy.

The term “alkoxy-alkyl” means a straight-chain or branched-chain saturated aliphatic hydrocarbon radical in which an alkoxy group is substituted for an alkyl hydrogen atom. An example of an alkoxy-alkyl group is 2-methoxyethyl.

The term “haloalkyl” means an alkyl group as defined above wherein one or more hydrogen atoms are replaced by fluorine, chlorine, bromine or iodine. When more than one hydrogen atom is replaced, the replacing halogen atoms may be the same or different. Examples of haloalkyl groups include fluoromethyl, difluoromethyl, trifluoromethyl, chlorodifluoromethyl, 2,2,2-trifluoroethyl and 3-bromopropyl.

The term “aryl” means a phenyl or naphthyl group.

The term “aryl-alkyl” means a straight-chain or branched-chain saturated aliphatic hydrocarbon radical in which an aryl group is substituted for an alkyl hydrogen atom. An example of an aryl-alkyl group is benzyl.

The term “cycloalkyl” means a saturated monocyclic or polycyclic carbocyclic ring containing the specified number of carbon atoms. Examples of monocyclic cycloalkyl groups include cyclopropyl, cyclobutyl, cyclopentyl and cyclohexyl. Examples of polycyclic cycloalkyl groups include bicyclo[2,2,1]heptyl and bicyclo[3,2,1]octyl.

The term “optionally substituted” with reference to an alkyl or aryl group means that a hydrogen atom of the alkyl or aryl group may be replaced by one of the groups listed. The substitution may be made at any position within the alkyl or aryl group. When the optional substitution is with “one or more groups” then any number of hydrogen atoms of the alkyl or aryl group, up to a maximum equal to the number of hydrogens present in the alkyl or aryl group, may be replaced, and each replacement is independent of the others.

The term “enantiomeric excess”, sometimes abbreviated as “e.e.”, is a measure, for a given sample, of the excess of one enantiomer in excess of its antipode and is expressed as a percentage. Enantiomeric excess is defined as:

100×(er−1)/(er+1)

where “er” is the ratio of the more abundant enantiomer to the less abundant enantiomer.

5-Hydroxy-4-(2-methylpropyl)-3,4-dihydro-5H-2-furanone (I^{A}) is a convenient intermediate in the manufacture of 3-aminomethyl-5-methylhexanoic acid (II). Treatment of racemic (I^{A}) with ammonia in the presence of a chemical reducing agent provides racemic 3-aminomethyl-5-methylhexanoic acid ((R/S)-II) (see WO2008/127646A2). The reaction is presumed to involve the ring-opened isomer (I^{B}).

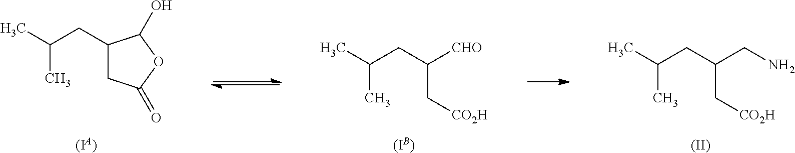

Reductive amination with an amine donor in the presence of a transaminase enzyme can provide enantiomerically enriched 3-aminomethyl-5-methylhexanoic acid. Suitable amine donors are primary amines such as mono-alkylamines, particularly isopropylamine, and α-amino acids.

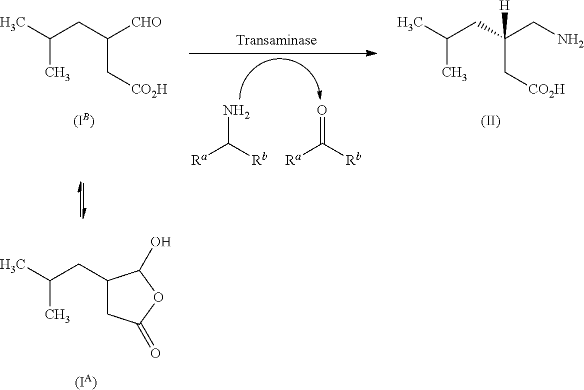

The reaction of (I^{A}) with a suitable amine dehydrogenase/imine reductase in the presence of ammonia can also be a suitable route to pregabalin. A co-factor such as NADH or NADPH may be needed in a stoichiometric amount, or a second oxidoreductase, such as formate dehydrogenase, may be included to recycle the co-factor.

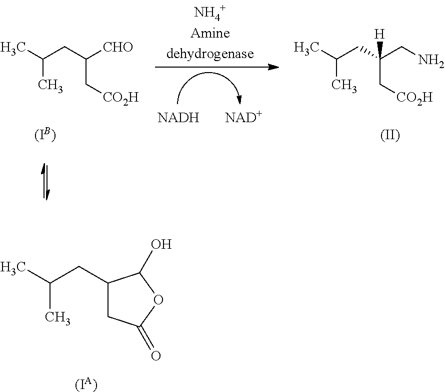

Where the dihydrofuranone (I^{A}) has a defined stereochemistry at the C-4 position, this stereochemistry may be preserved during the reductive amination reaction. Since Pregabalin has the (S)-stereochemistry it may be preferred to have this stereochemistry already present in the dihydrofuranone.

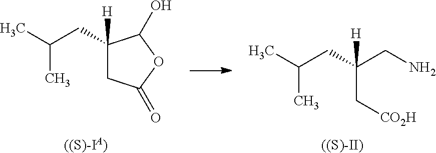

Alternatively, the dihydrofuranone (I^{A}) may be used as in racemic form. The desired stereoisomer of the product may then be obtained either by carrying out the reductive amination reaction under conditions that allow for the stereoselective formation of a single enantiomer, such as by carrying out the transformation in the presence of a transaminase enzyme, or by subjecting the product to a separate resolution step, such as by crystallization with a chiral acid or base.

The dihydrofuranone (I^{A}) may be conveniently prepared in racemic or enantiomerically enriched form using the methods set out below.

In a first method, the dihydrofuranone (I^{A}) is prepared by reduction of 5-hydroxy-4-(2-methyl-1-propenyl)-5H-2-furanone (VI^{A}).

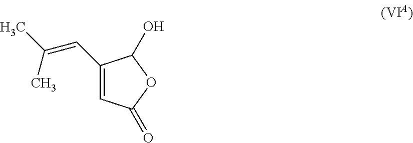

The reduction may conveniently be accomplished by hydrogenation in the presence of a suitable catalyst. Suitable catalysts include homogeneous and heterogeneous catalysts. The catalyst typically comprises a transition metal such as palladium, platinum, rhodium, ruthenium or nickel, or a salt or oxide thereof. Heterogeneous catalysts include finely divided metals and substrate-supported metals and metal oxides, where the substrate may be carbon, silica, alumina or any other suitable inert material. Homogeneous catalysts include phosphine ligand complexes of transition metals. When the phosphine ligand is chiral then the catalyst is chiral. When an achiral catalyst is used, then the product is the racemic dihydrofuranone (I^{A}). The use of a chiral catalyst may provide the dihydrofuranone (I^{A}) in an enantioselective manner.

The selectivity of the hydrogenation reaction, and the overall yield, maybe improved when the reaction is carried out in an alkaline medium. Without being bound by theory, it is thought that in the presence of a base the furanone exists predominantly as the ring-opened salt (IX).

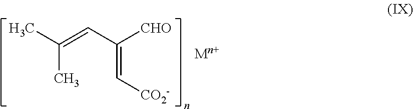

Any suitable base may be used provided that it does not interfere with the hydrogenation process, such as by poisoning the catalyst. Examples of suitable bases include alkali (such as Li, Na, K and Rb) and alkaline earth metal (such as Ca and Mg) oxides, hydroxides, carbonates and bicarbonates. Other metal salts, such as zinc salts, may also be used. Alkali metal salts may be preferred due to their good solubility and/or low toxicity. Amine bases such as ammonia, and primary, secondary and tertiary amines may be used to prepare ammonium salts. Tetra-alkylammonium hydroxide may also be used, leading to the formation of tetra-alkylammonium salts.

Hydrogenation of the salt of formula (IX) provides a salt of formula (X).

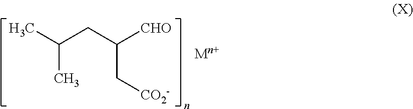

The dihydrofuranone (I^{A}) is recovered, after the hydrogenation reaction, by treatment of this salt with a suitable acid. Alternatively, the salt may be converted directly to 3-aminomethyl-5-methylhexanoic acid (II) by treatment with a transaminase or amine oxidase/imine reductase enzyme. In this case it may be preferable to use the ammonium salt (M⁺=NH₄⁺) or a primary alkylammonium salt (M⁺=alkyl-NH₃⁺) since the ammonium or alkylammonium ion provides the co-substrate for the enzyme. The use of the isopropylammonium salt in combination with a transaminase enzyme is a preferred example.

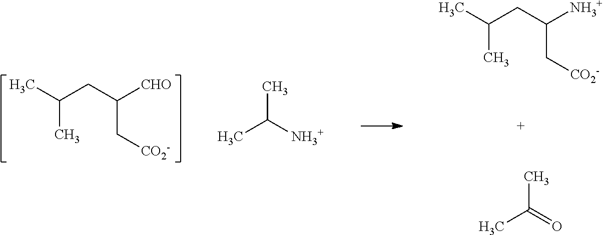

Furanones of formula (VI) wherein R is other than hydrogen may also be reduced by hydrogenation. Where R is an alkyl, haloalkyl, alkoxyalkyl alkenyl, cycloalkyl, cycloalkyl-alkyl, aryl or aryl-alkyl group, these furanones may be prepared from the compound of formula (VI^{A}) by reaction with an alcohol R—OH in the presence of an acid catalyst. Where R is R¹—C(O)— the furanones may be prepared from the compound of formula (VI^{A}) by reaction with an acid anhydride (R¹—C(O))₂O or acid chloride R¹—C(O)—Cl, optionally in the presence of a base, for example a tertiary amine. Where R is R²—SO₂— the furanones may be prepared from the compound of formula (VI^{A}) by reaction with a sulfonyl chloride R²—SO₂—Cl, optionally in the presence of a base, for example a tertiary amine.

Following the hydrogenation step the dihydrofuranone of formula (I^{A}) can be generated from the reduction product by treatment with acid (where R is an alkyl, haloalkyl, alkoxyalkyl alkenyl, cycloalkyl, cycloalkyl-alkyl, aryl or aryl-alkyl group) or base (where R is R¹—C(O)— or R²—SO₂—).

The use of a chiral R group may provide a chiral hydrogenation product without the need for a chiral catalyst.

For example, the furanone (VI^{A}) is reacted with a chiral alcohol R*—OH to afford a chiral ether derivative (VI^{B}).

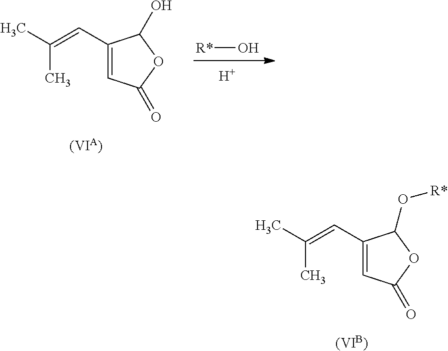

Suitable chiral alcohols may include α-aryl alcohols such as 1-phenylethanol and 1-naphthylethanol, as well as terpene alcohols such as menthol and borneol. Hydrogenation of derivative (VI^{B}) may proceed in an enantioselective manner, and treatment of the resulting product with a suitable acid and in the presence of water then provides the dihydrofuranone (I^{A}) in chiral form.

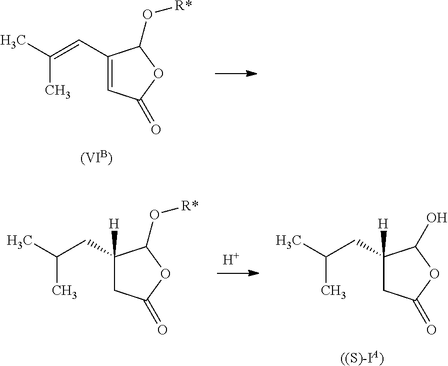

The furanone (VI^{A}) may be prepared from a compound of formula (VII)

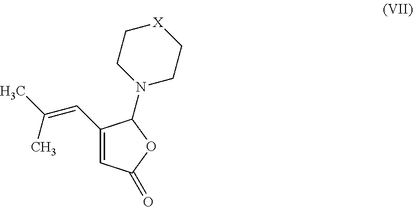

wherein —X— represents a single bond, —CH₂—, —O—, —NH—, —N((C₁-C₃)alkyl)-, —N(benzyl)-, or

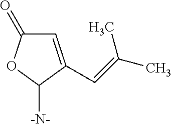

by treating the compound of formula (VII) with water in the presence of an acid catalyst. Suitable acids include mineral acids such as sulphuric acid. Alternatively, the compound of formula (VII) can be treated with an alcohol R—OH to provide directly a compound of formula (VI) wherein R is an alkyl, haloalkyl, alkoxyalkyl alkenyl, cycloalkyl, cycloalkyl-alkyl, aryl or aryl-alkyl group.

The compounds of formula (VII) can be prepared by the reaction of a dienamine of formula (VIII).

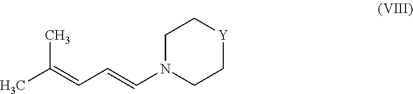

wherein —Y— represents a single bond, —CH₂—, —O—; —NH—, —N((C₁-C₃)alkyl)-, —N(benzyl)-, or

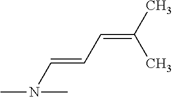

with glyoxylic acid or its hydrate.

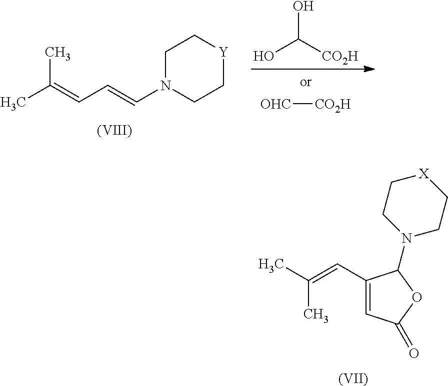

It will be understood that —X— in formula (VII) corresponds to —Y— in the starting material of formula (VIII), except that when —Y— is

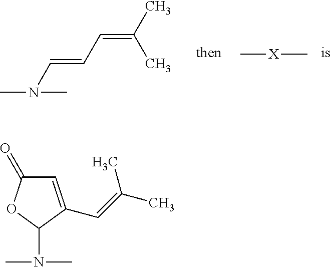

The compounds of formula (VIII) wherein —Y— represents a single bond or —CH₂— have been prepared by the reaction of 4-methyl-2-pentenal with pyrrolidine or piperidine (Kienzle, F. et al., Helv. Chim. Acta 1985, 68(5), 1133-39). Other compounds of formula (VIII) may be prepared analogously.

Alternatively, condensation of isobutyraldehyde and acetaldehyde in the presence of a suitable amine such as pyrrolidine, piperidine or morpholine with catalytic acid in a solvent such as acetonitrile provides the dienamine derivatives (VIII).

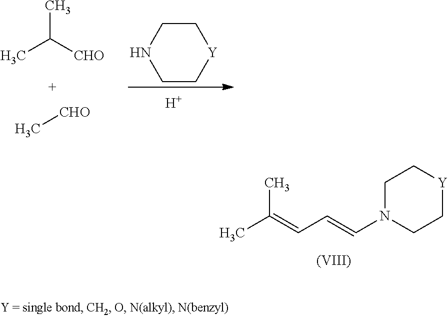

If piperazine is used as the amine then a bis-dienamine is obtained.

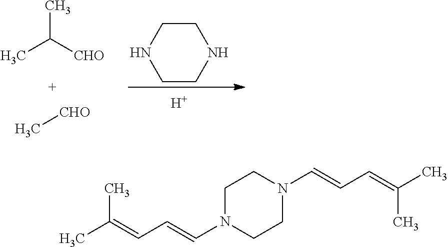

The compound of formula (VIII) wherein Y is NH may be obtained by using mono-protected piperazine as the amine, followed by a deprotection step.

Acyclic secondary amines such as diethylamine and di-isopropylamine may also be used, but cyclic secondary amines are preferred.

This mode of reaction of isobutyraldehyde and acetaldehyde differs from the direct base catalysed reaction (eg using potassium carbonate as base: UK patent GB834100) which only gives the adduct 2,2-dimethyl-3-hydroxybutanal where isobutyraldehyde acts as a nucleophile.

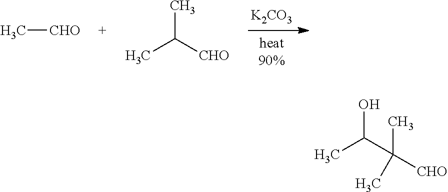

Without wishing to be bound by any particular theory, it is postulated that both the acetaldehyde and the isobutyraldehyde are initially converted to their enamine derivatives. In the presence of the acid catalyst, the more basic isobutyraldehyde enamine is converted to its iminium ion. This electrophilic species reacts preferentially with the less sterically hindered nucleophile, which is the acetaldehyde enamine—this ensures reaction the desired way around.

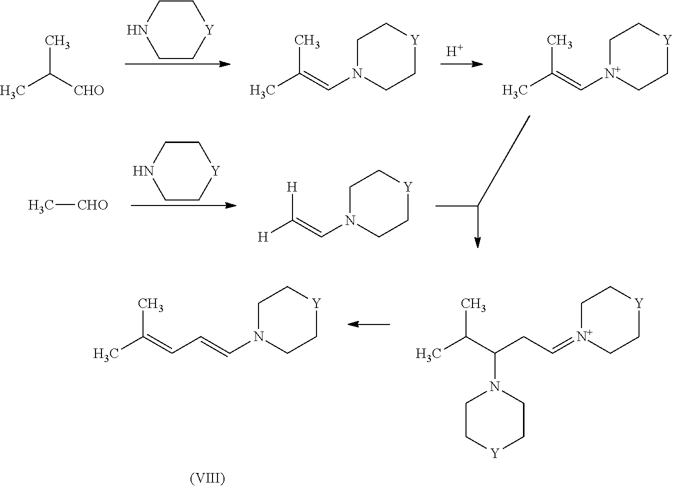

The method of the present invention is more economical and more amenable to scale-up than the literature methods of effecting this ‘umpolung’ of normal acetaldehyde reactivity, in which acetaldehyde is converted to an O-silylated enol derivative and coupled with the isobutyraldehyde under Mukiyama aldol conditions, In the present invention, both coupling partners are activated simultaneously—electronic and steric effects directing the observed reactivity pattern. The other advantage is that the product dienamine is the desired ‘activated’ form of 4-methyl-2-pentenal for reaction with glyoxylic acid to form the desired 5-aminofuranones (VII).

These dienamine derivatives of formula (VIII) may be isolated and purified, or alternatively they can be treated directly with glyoxylic acid (or its hydrate), which provides furanone derivative (VII) directly.

The furanone derivatives (VII) may be isolated and purified. Treatment with aqueous acid then provides furanone (VI^{A}).

Overall, the transformations set out above provide a short route for the manufacture of pregabalin using inexpensive and safe starting materials.

In an alternative embodiment, the dihydrofuranone (I) is prepared from 3-isobutylidene-2-oxopentanedioic acid (XII), which is readily obtained by the condensation of isobutyraldehyde with 2-oxopentanedioic acid (α-ketoglutaric acid) (XIV).

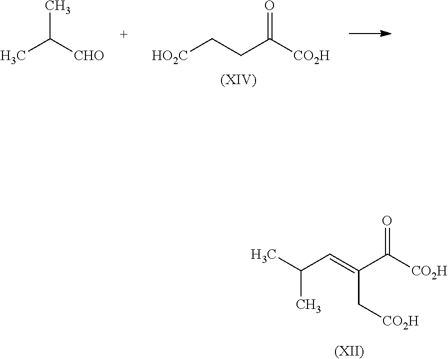

Conversion of diacid (XII) to dihydrofuranone (I) requires a decarboxylation step and a reduction step. These two process steps may be carried out separately, in which case either the decarboxylation step or the reduction step may be the first step, or the two processes may be carried out simultaneously. When the reduction step is carried out first, the intermediate (XV) will be produced. When the decarboxylation step is carried out first the intermediate (XVI) will be produced.

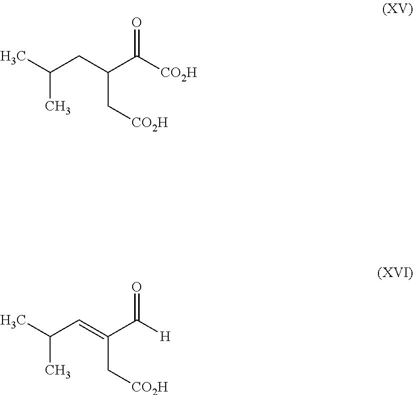

The reduction step may be carried out chemically, such as by hydrogenation, but is preferably achieved using an enzyme-mediated reduction, such as by treating with an enoate reductase enzyme. The decarboxylation step is preferably performed by treating the compound with a decarboxylase enzyme.

Where enzyme-mediated transformations are contemplated, the enzyme may be an isolated enzyme, including an enzyme immobilized on a carrier, it may be a partially isolated enzyme preparation such as a cell homogenate, or it may be a non-isolated enzyme, in which case a whole-cell preparation is used. Cells may include those which express the desired enzyme naturally, and cells that have been manipulated so as to express the desired enzyme.

The enzyme-mediated reductive amination of the compound of formula (I^{A}) is reversible, and so treatment of 3-aminomethyl-5-methylhexanoic acid (II) with a transaminase enzyme or an amine oxidase/imine reductase enzyme can lead to the formation of the dihydrofuranone (I^{A}). The ring-opened isomer of this is compound (I^{B}), which is epimerizable. In view of this, it is possible to convert ((R)-II) into ((S)-II), or to increase the optical purity of a mixture of ((R)-II) and ((S)-II) using such an enzyme.

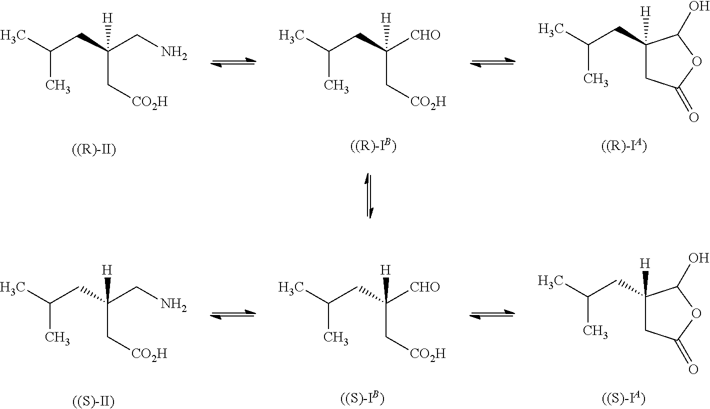

### EXAMPLES

The invention is illustrated by the following non-limiting examples in which the following abbreviations and definitions are used:
bp Boiling point
CPME Cyclopentyl methyl ether
d Doublet
DIW De-ionised water
dd Doublet of doublets
eq or eq. Equivalent
e.e. or ee Enantiomeric excess
ES⁺ Positive mode electrospray ionization
EtOAc Ethyl Acetate
EtOH Ethanol
GC Gas chromatography
GC/MS Gas chromatography/Mass spectroscopy
HOAc Acetic acid
HPLC High Performance Liquid Chromatography
Hr or h Hour
¹H NMR Proton Nuclear Magnetic Resonance Spectroscopy
L Litre
LCMS Liquid chromatography/Mass spectroscopy
m Multiplet
mbar Millibar
MeOH Methanol
min Minute
mL Millilitre
mmol Millimole
mol Mole
Mp Melting point
MTBE Methyl tert-butyl Ether
NAD⁺ Nicotinamide adenine dinucleotide (oxidised)
NADP⁺ Nicotinamide adenine dinucleotide phosphate (oxidised)
NADH Nicotinamide adenine dinucleotide (reduced)
NADPH Nicotinamide adenine dinucleotide phosphate (reduced)
PLP Pyridoxal phosphate
ppm Parts per million
pTsOH Para-toluenesulfonic acid
q Quartet
qNMR Quantitative nuclear magnetic resonance spectroscopy
R_{f} Retention factor
RT Room temperature
s Singlet
t Triplet
ThDP Thiamine diphosphate
TLC Thin layer chromatography
TsOH Toluenesulfonic acid (=pTsOH)
UPLC Ultra Performance Liquid Chromatography
XRD X-ray diffraction crystallography
δ Chemical shift

Commercial chemicals were used as received unless stated otherwise. Thin layer chromatography was performed on pre-coated plastic plates (Merck silica 60F254), and visualised using UV light and KMnO₄ dip. Proton (¹H) and carbon (¹³C) NMR spectra were recorded on a Varian INOVA 300 MHz spectrometer. Chemical shifts are quoted relative to tetramethylsilane and referenced to residual solvent peaks as appropriate. Unless otherwise indicated, chiral HPLC analysis was performed using an Agilent 1200 HPLC system and data was processed using the Chemstation software or with a Varian semiprep/analytical HPLC using Galaxie software.

### Example 1

Preparation of 4-(2-methyl-1-propenyl)-5-morpholino-5H-2-furanone from 4-methyl-2-pentenal

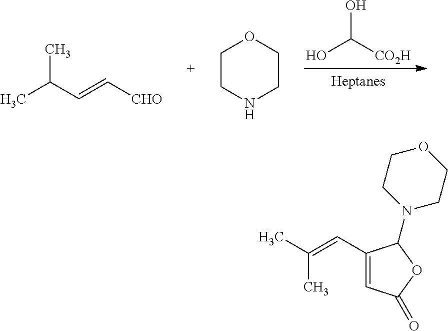

A 50% solution of glyoxylic acid in water (29.6 g, 0.2 mol) was added to a biphasic stirred mixture of morpholine (17.8 g, 0.2 mol) and heptanes (75 mL), which had been pre-cooled to 0-10° C. The temperature was kept less than 10° C. The mixture was warmed to 20° C. and 4-methyl-2-pentenal (19.6 g, 0.2 mol) was added. The mixture was stirred at 45° C. for 20 h. A large quantity of solid was formed. Water (100 mL) was added at ambient temperature and the mixture stirred for 2 h. The mixture was extracted with cyclopentyl methyl ether (100 mL) and the organic solution washed twice with water (100 mL) and concentrated to leave 30.4 g of crude product. This solid was purified by recrystallisation from methanol (100 mL) to afford 17.7 g (40%) of pure 4-(2-methyl-1-propenyl)-5-morpholino-5H-2-furanone.

GC/MS: m/z=223

¹H NMR: δ 6.0 (s, 1H); 5.9 (s, 1H); 5.5 (s, 1H), 3.7 (d, 4H), 2.7 (d, 4H), 2.00 (s, 3H), 1.95 (s, 3H).

### Example 2

### Preparation of 4-(4-methyl-1,3-pentadien-1-yl)morpholine

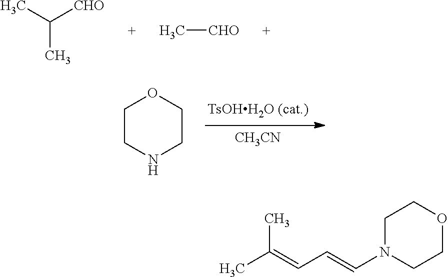

Isobutyraldehyde (46.90 g, 0.65 mol, 1.43 eq.) was stirred in acetonitrile (300 mL). Morpholine (56.63 g, 0.65 mol, 1.43 eq.) followed by pTsOH (8.63 g, 0.1 equiv) were slowly added at room temperature to the isobutyraldehyde solution. A solution of acetaldehyde (20 g, 0.454 mol) in acetonitrile (100 mL) was added drop-wise over 1 hr with internal temperature monitoring at 50° C. After complete addition the mixture was stirred for 30 min at 50° C. and then cooled to room temperature before evaporation of the solvent in vacuo to give an orange oil (123.3 g). Assay of the crude by quantitative NMR using benzyl benzoate as the internal standard was 40% which give 65% yield of the desired dienamine.

GC/MS: m/z=167

¹H NMR: δ 6.01 (d, 1H); 5.69 (d, 1H); 5.31 (dd, 1H), 3.70 (m, 4H), 2.89 (m, 4H), 1.71 (s, 3H), 1.63 (s, 3H).

### Example 3

### Preparation of 4-(2-methyl-1-propenyl)-5-morpholino-5H-2-furanone from crude 4-(4-methyl-1,3-pentadien-1-yl)morpholine

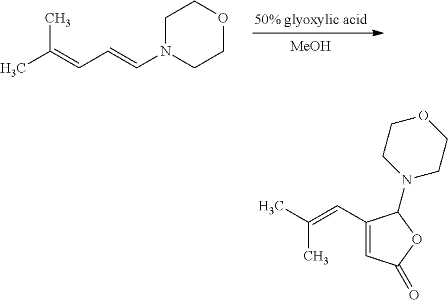 [SMILES: "MeOH" ; CC(C)=C/C=C/N1CCOCC1 ; CC(C)=CC1=CC(=O)OC1N1CCOCC1]

The crude 4-(4-methyl-1,3-pentadien-1-yl)morpholine dienamine of example 2 (123.3 g, 40% assay) was dissolved in methanol (300 mL) at room temperature. On complete dissolution, glyoxylic acid (50 wt %, 60 g, 1.1 eq) was added and the resultant biphasic mixture stirred at 50° C. for 18 h. The reaction mixture was cooled to room temperature and the solvent removed by rotary evaporation. The residue was partitioned between ethyl acetate (200 mL) and saturated sodium carbonate solution (200 mL). The aqueous phase was extracted with ethyl acetate (100 mL) and combined organics washed with brine and evaporated to a thick oil which solidified on standing (89.0 g, 67% assay by qNMR, 60% yield).

GC/MS: m/z=223

NMR: as example 1.

### Example 4

### Preparation of 4-(2-methyl-1-propenyl)-5-morpholino-5H-2-furanone in one pot from isobutyraldehyde and acetaldehyde

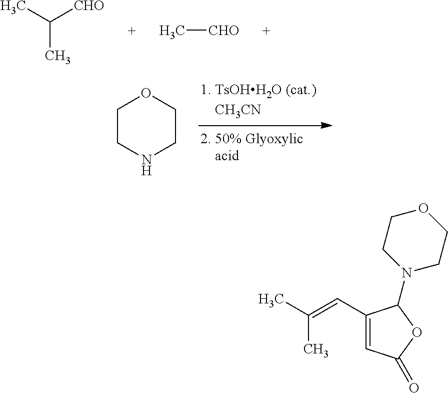

Isobutyraldehyde (102.90 g, 1.43 mol) was stirred in acetonitrile (600 mL). Morpholine (124.3 g, 1.43 mol) followed by pTsOH (19.0 g, 0.1 equiv) were slowly added at room temperature to the isobutyraldehyde solution. A solution of acetaldehyde (44.05 g, 1.0 mol) in acetonitrile (150 mL) was added drop-wise over 1 hr with internal temperature monitoring at 50° C. After complete addition the mixture was stirred for 30 min at 50° C. and then cooled to <10° C. Glyoxylic acid (50 wt %, 211.3 g, 1.43 mol) was added and the resultant biphasic mixture stirred at 50° C. for 18 h. The reaction mixture was cooled to room temperature and the solvent removed by rotary evaporation. The residue was partitioned between ethyl acetate (1 L) and saturated sodium carbonate solution (1 L). The aqueous was extracted with ethyl acetate and combined organics washed with brine and evaporated to a thick oil which solidified on standing (166 g). The crude product was triturated with MTBE at room temperature. The product is collected by filtration and washed with MTBE to give pure 4-(2-methyl-1-propenyl)-5-morpholino-5H-2-furanone (84 g, 37% yield) identical to the material prepared in Example 1. Analysis of the crude solid (166 g) indicated ca 50% yield.

### Example 5

Preparation of 1,4-bis-(4-methyl-1,3-pentadien-1-yl)piperazine

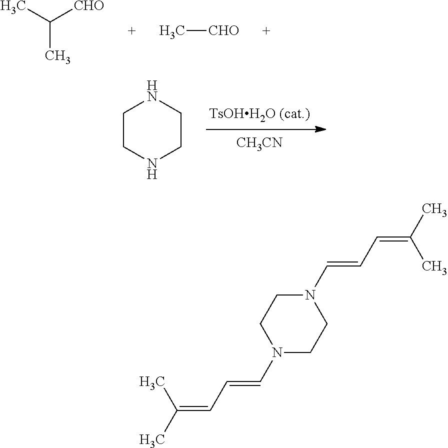

A solution of piperazine (43.0 g, 0.5 mol) and 4-toluenesulfonic acid monohydrate (4.4 g, 0.023 mol) in acetonitrile (600 mL) was prepared. This solution was heated to 50° C. and isobutyraldehyde (100 mL, 1.10 mol) added over 10 minutes. The solution went red-orange in colour and a transient white precipitate occurred. A solution of acetaldehyde (30.8 g, 0.70 mol) in acetonitrile (30 mL) was then added via syringe pump over 3 h at 50° C. A suspension was formed which was stirred at 50° C. for 0.5 h. The solvent was removed and the residual solid isolated from methanol (500 mL) at −5° C. It was filtered and washed with chilled methanol and dried to afford 52.9 g (60%) of 1,4-bis-(4-methyl-1,3-pentadien-1-yl)piperazine.

GC/MS: m/z=246.

¹H NMR: δ 6.00 (d, 2H); 5.70 (d, 2H); 5.28 (dd, 2H); 2.92 (s, 8H); 1.73 (s, 6H); 1.68 (s, 6H). ¹³C NMR (CDCl₃): 140.6 (C), 126.3 (CH), 123.4 (CH), 100.2 (CH), 48.2 (CH₂), 25.8 (CH₃), 18.1 (CH₃).

### Example 6

### Preparation of 5,5′-(piperazine-1,4-diyl)bis(4-(2-methylprop-1-en-1-yl)furan-2(5H)-one) from 1,4-bis-(4-methyl-1,3-pentadien-1-yl)piperazine

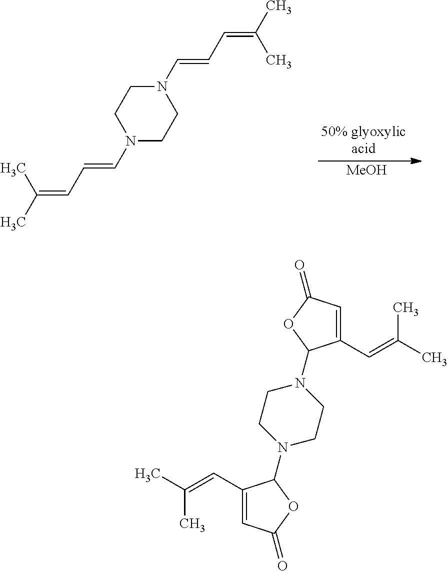

1,4-bis-(4-Methyl-1,3-pentadien-1-yl)piperazine (37.3 g, 0.151 mol; see Example 5) was charged to methanol (300 mL). The temperature was adjusted to 34° C. and a 50% solution of glyoxylic acid in water (44.9 g, 0.302 mol) was added rapidly (over 5 minutes). The resulting suspension was stirred at 45° C. for 15 h; cooled to 0-10° C. for 2 h and filtered. The solid was washed with methanol (100 mL) and dried to afford pure 5,5′-(piperazine-1,4-diyl)bis(4-(2-methylprop-1-en-1-yl)furan-2(5H)-one) (32.3 g, 60%). An extra 5% could be obtained from the mother-liquor by concentration.

¹H NMR: [symbol] 5.96 (d, 2H), 5.87 (d, 2H), 5.56 (m, 2H), 2.71 (s, 8H), 2.07-1.90 (m, 12H). ¹³C NMR (CDCl₃): 172.3 (C), 159.2 (C), 150.9 (C), 116.6 (CH), 115.4 (CH), 99.2 (CH), 46.7 (CH₂), 28.2 (CH₃), 21.4 (CH₃).

This reaction can also be conducted in isopropanol, acetonitrile-water, toluene-water or in heptane water with similar yields.

### Example 7

### Preparation of 5,5′-(piperazine-1,4-diyl)bis(4-(2-methylprop-1-en-1-yl)furan-2(5H)-one) in one pot from isobutyraldehyde and acetaldehyde

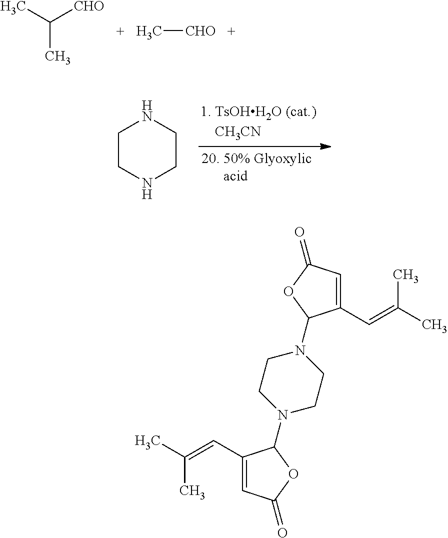

Tosic acid (6.5 g, 0.03 mol) was charged to a solution of piperazine (59 g, 0.69 mol) in acetonitrile (240 mL). The reaction was heated to 50° C. and agitated until dissolution of solids was observed, before addition of isobutyraldehyde (138 mL, 1.51 mol). The reaction was held at 50° C. before a solution of acetaldehyde (60 mL; 1.07 mol) in acetonitrile (30 mL) was charged to the vessel over 3 hrs via syringe pump. On complete addition, the reaction was stirred for a further 30 minutes before a 50% w/w solution of glyoxylic acid in water (148 g, 1.0 mol) was added over 5 min to the reaction mixture followed by water (50 ml). The reaction was then heated to 70° C. for 2 h, then to 50° C. overnight. The reaction was then cooled to 5° C. and held for 30 minutes. 5,5′-(Piperazine-1,4-diyl)bis(4-(2-methylprop-1-en-1-yl)furan-2(5H)-one) precipitated and was isolated by filtration and washed with acetonitrile (2×100 mL) to give the desired product (126 g, 93.5% assay, 66% yield from acetaldehyde).

### Example 8

### Solvent free preparation of 5,5′-(piperazine-1,4-diyl)bis(4-(2-methylprop-1-en-1-yl)furan-2(5H)-one)

Isobutyraldehyde (79 g, 100 mL, 1.1 mol) was charged to a 3-necked round bottom flask and argon was flushed through the system. Piperazine (43 g, 0.5 mol) and TsOH—H₂O (4.4 g; 0.023 mol) were divided into 4 equal portions containing piperazine (10.75 g) and TsOH—H₂O (1.1 g). Addition of the first portion of piperazine (10.75 g) resulted in exotherm from 24° C. to 35° C. This was followed by the first portion of TsOH (1.1 g). The reaction was agitated until all the piperazine had dissolved (t=35° C.), after which the second portion of piperazine (10.75 g) was added, followed by TsOH (1.1 g) (t=41° C.). Stirring was continued until all of piperazine had dissolved (t=41° C.), then the third portion of piperazine (10.75 g) was added, followed by TsOH (1.1 g). After the 3^{rd} charge a clear solution was generated and then the fourth portion of piperazine (10.75 g) & TsOH—H₂O were added, followed by TsOH (1.1 g) (t=52° C.). On complete addition the reaction was stirred at 50° C. for 30 min.

Another flask (50 mL) was charged with acetaldehyde (30.8 g, 39 mL, 0.7 mol) and placed in an ice-water bath (0-2° C.). The acetaldehyde flask was connected to the 3-necked flask by cannula via septa. A stream of argon was then passed through the acetaldehyde flask at the rate which ensures that the addition of the acetaldehyde was complete within 3 h. After all acetaldehyde was added, the reaction was stirred for 30 min at 50° C., then cooled to 40° C. and 50% glyoxylic acid (104 g, 78 mL) was added dropwise over 45 min at a rate to keep temperature below 50° C. Water (100 mL) was then added and the reaction was heated at 70° C. for 5 h. The reaction was cooled to 4° C. in an ice-water bath. The precipitated product was filtered and filter cake was washed with cold water (100 mL). After drying in vacuo at 50° C. 88.9 g (71%) of desired product was obtained. Precipitate contains 78.5% of the title compound (HPLC assay).

### Example 9

### Alternative preparation of 5,5′-(piperazine-1,4-diyl)bis(4-(2-methylprop-1-en-1-yl)furan-2(5H)-one) with simultaneous addition of both aldehydes

Piperazine (236.6 g) was charged to a clean 3 L dry vessel fitted with thermometer, addition funnel (100 mL) and reflux condenser. pTsOH (25.3 g) was charged to vessel followed by acetonitrile (550 mL). The agitator was started and the vessel was inserted with nitrogen. Isobutyraldehyde (150 mL, 27% of the total charge) was charged to the agitated piperazine/pTsOH slurry and a temperature rise to ca 43° C. was observed. The white suspension was then heated to 50° C. (+/−5° C.). A pre-mixed chilled solution of isobutyraldehyde (400 mL, 73% of total charge) and chilled acetaldehyde (260 mL) were charged to a clean, dry 1 L vessel and this mixture was maintained in an ice bath. The isobutyraldehyde/acetaldehyde mixture was charged to the contents of the 3 L vessel in 100 mL aliquots over 5-6 h at 50° C. The contents of the reaction flask changed from a white suspension to a wine red solution and finally an orange suspension during the addition of the acetaldehyde. After complete addition the suspension was agitated at 50° C. for ca. 0.5 to 1.0 h.

Aqueous glyoxylic acid (50% wt/wt) solution was charged to the suspension over 10-15 min and the temperature rose to ca 75° C. 5,5′-(Piperazine-1,4-diyl)bis(4-(2-methylprop-1-en-1-yl)furan-2(5H)-one) was observed to crystallise from solution. The suspension was stirred at 70° C. (+/5° C.) for 6 h, then cooled with stirring to ambient. The batch was then cooled to −5 to 0° C. for and held for 3-4 h before the suspension was filtered and the cake washed with chilled methanol (1×500 mL) then 2×500 mL of methanol at ambient temperature. The washed product was dried in a vacuum oven at 40-50° C. to constant weight to afford 474 g of 98% pure 5,5′-(piperazine-1,4-diyl)bis(4-(2-methylprop-1-en-1-yl)furan-2(5H)-one) (70%).

### Example 10

### Preparation of 5-hydroxy-4-(2-methylprop-1-en-1-yl)furan-2(5H)-one from 4-(2-methyl-1-propenyl)-5-morpholino-2(5H)-furanone or 5,5′-(piperazine-1,4-diyl)bis(4-(2-methylprop-1-en-1-yl)furan-2(5H)-one)

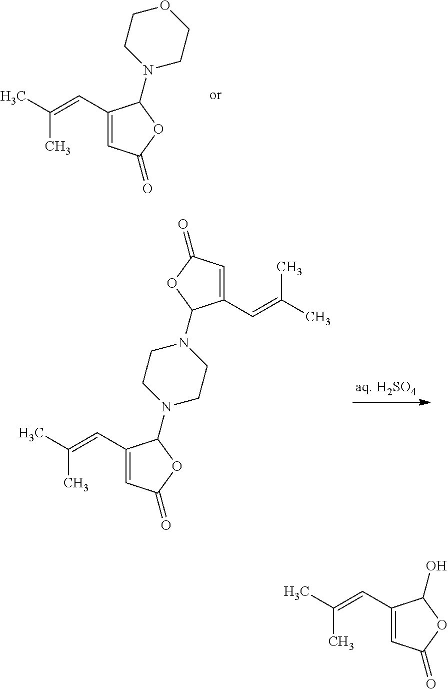

A 10% w/w aqueous sulphuric acid solution (110 g) was charged to 4-(2-methyl-1-propenyl)-5-morpholino-2(5H)-furanone (20.0 g, 0.896 mol) and the mixture was stirred at reflux for 4 h or until TLC (100:3 CPME:HOAc) indicated reaction completion. The mixture remained a suspension at all times. It was then cooled to 5° C., held for 2 h, and filtered. The white solid was washed with water and dried to afford 5-hydroxy-4-(2-methylprop-1-en-1-yl)furan-2(5H)-one (12.9 g, 93%).

Alternatively, a 10% w/w aqueous solution of sulphuric acid (412 g) was charged to a vessel containing 5,5′-(piperazine-1,4-diyl)bis(4-(2-methylprop-1-en-1-yl)furan-2(5H)-one) (60 g, 0.167 mol). The contents were agitated and then heated to reflux. The reaction was held at reflux until the starting material was consumed, which was determined by dissolution. On completion of reaction the batch was cooled to 35° C. at which point the target compound began to crystallise. The slurry was further cooled to 0-5° C. and then transferred to a filter. The product was filtered, washed with water (2×100 mL) and then dried under vacuo at <50° C., to give 5-hydroxy-4-(2-methylprop-1-en-1-yl)furan-2(5H)-one (42.0 g, 81%).

GC/MS: m/z=154

¹H NMR (CDCl₃): δ 6.10 (s, 1H); 5.92 (s, 1H); 5.88 (s, 1H); 5.35 (bs, 1H, O—H); 1.98 (s, 3H); 1.93 (s, 3H). ¹³C NMR (CDCl₃): 172.8 (C), 161.4 (C), 152.3 (C), 115.3 (CH), 114.9 (CH), 99.5 (CH), 28.2 (CH₃), 21.4 (CH₃).

### Example 11

### Preparation of 5-hydroxy-4-(2-methylprop-1-en-1-yl)furan-2(5H)-one in one pot from isobutyraldehyde and acetaldehyde

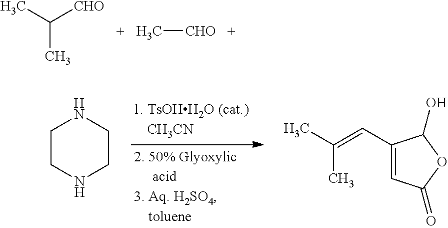

A mixture of piperazine (43.0 g, 0.5 mol) and isobutyraldehyde (200 mL) were refluxed under Dean-Stark until the theoretical amount of water (18 mL) collected. The excess isobutyraldehyde was distilled off to leave a crystalline mass of 1,4 bis(2-methylpropen-1-yl)piperazine. This was dissolved in acetonitrile (1.2 L) and extra isobutyraldehyde (100 mL, 1.0 mol) added. Extra piperazine (4.4 g) was added followed by p-toluenesulfonic acid (4.4 g, 23.2 mmol). The temperature was adjusted to 40° C. and a solution of acetaldehyde (44 g, 1.0 mol) in acetonitrile (40 mL) was added over 4 h. The reaction mixture was stirred at 40° C. for 1 h and at ambient temperature for 4 h. The acetonitrile was distilled off and the residue suspended in toluene (400 mL). A mixture of 50% glyoxylic acid (148 g) and water (150 mL) was added over 0.5 h. The mixture was then stirred at 45° C. for 15 h. There was a solid precipitate. Dilute sulphuric acid (10%, 1 L) was added and the mixture refluxed for 3 h. A biphasic mixture resulted. The toluene phase was separated. It contained ca 50% yield (based on acetaldehyde) of 5-hydroxy-4-(2-methylpropen-1-yl)-2-furanone by analysis

### Example 12

### Preparation of 5-methoxy-4-(2-methylprop-1-en-1-yl)furan-2(5H)-one

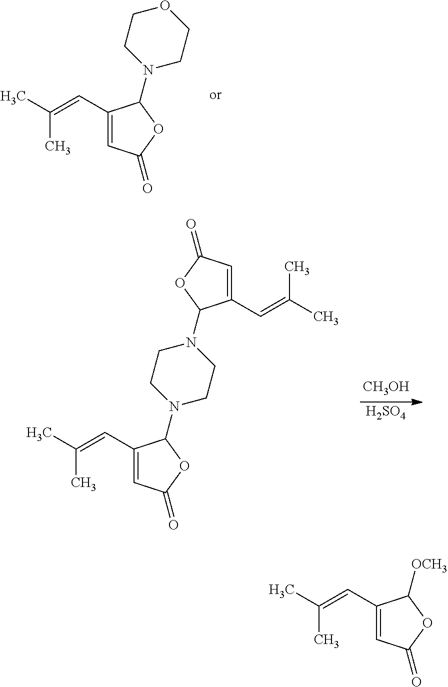

4-(2-Methyl-1-propenyl)-5-morpholino-2(5H)-furanone (7.70 g, 34.5 mmol) was stirred in MeOH (50 mL) with H₂SO₄ (4 mL, 2.2 eq.) at reflux for 3 h until disappearance of the starting material by GC. The reaction mixture was cooled to room temperature. The solvent was then evaporated in vacuo and the residue was diluted with EtOAc (50 mL). The organic phase was washed with H₂O (3×50 mL). Solvent was then evaporated in vacuo to give 5-methoxy-4-(2-methylprop-1-en-1-yl)furan-2(5H)-one as an orange oil (5.50 g, 95%) which can be used without purification.

Alternatively, 5,5′-(piperazine-1,4-diyl)bis(4-(2-methylprop-1-en-1-yl)furan-2(5H)-one) (130 g, 363 mmol) and MeOH (690 mL) were charged in 2 L round bottom flask with mechanical agitation to form a suspension. Concentrated H₂SO₄ (43 mL, 762 mmol) was added dropwise with stirring at 20-25° C.—a slight exotherm was observed, and nature of the solids present changed from dispersed and easily mixed to a thicker slurry. The reaction mixture was refluxed for 5 h. After 0.5 h complete dissolution (orange liquid) was observed. After 5 h LCMS showed ˜99% of the desired product. The mixture was cooled to ambient and left for crystallization of piperazine sulfate. The sediments were filtered, and filter cake was washed with ice cold MeOH (400 mL). The filtrate was concentrated in vacuo (40° C. bath temperature), and the residue was partitioned between water (50 mL) and MTBE (300 mL). Water layer was separated and additionally extracted (2×300 mL of MTBE). Combined organic extracts were washed with saturated aq. NaHCO₃ (200 mL). MTBE layer was dried over Na₂SO₄ with stirring for 1.5 h, filtered and evaporated to give 5-methoxy-4-(2-methylprop-1-en-1-yl)furan-2(5H)-one as an orange oil (120 g, 98%).

Distilled at 0.2-0.3 mbar vacuum with by 100-105° C. GC/MS: m/z=168; ¹H NMR (400 MHz, CDCl₃) δ 5.59 (1H, s) 5.88-5.86 (1H, m) 5.75 (1H, d, J=0.6) 3.53 (3H, s) 2.01-2.00 (3H, m) 1.97-1.96 (3H, m). ¹³C NMR (CDCl₃): 171.4 (C), 159.2 (C), 151.9 (C), 115.8 (CH), 115.2 (CH), 104.4 (CH), 56.1 (CH₃), 28.2 (CH₃), 21.4 (CH₃).

The following compounds were prepared from 4-(2-methyl-1-propenyl)-5-morpholino-2(5H)-furanone according to the first of the above methods by replacing methanol with 3-methylbutanol (isoamyl alcohol), n-pentanol (amyl alcohol) or n-butanol:

### Example 12A

### 5-(3-Methylbutoxy)-4-(2-methylprop-1-en-1-yl)furan-2(5H)-one

Distilled at 0.4 mbar vacuum with by 139-140° C.; GC/MS: m/z=224; ¹H-NMR (400 MHz, CDCl₃) δ 5.94 (1H, s) 5.87-5.84 (1H, m) 5.80 (1H, s) 3.86-3.80 (1H, m) 3.70-3.64 (1H, m) 2.00 (3H, s) 1.96 (3H, s) 1.77-1.66 (1H, m) 1.57-1.50 (2H, m) 0.93-0.91 (3H, m) 0.91-0.89 (3H, m)

### Example 12B

### 4-(2-Methylprop-1-en-1-yl)-5-pentyloxyfuran-2(5H)-one

Distilled at 0.08 mbar vacuum with by 123-134° C.; GC/MS: m/z=224; ¹H-NMR (400 MHz, CDCl₃) δ 5.94 (s, 1H); 5.86 (s, 1H); 5.80 (s, 1H); 3.84-3.75 (m, 1H); 3.68-3.59 (m, 1H); 2.01 (s, 3H); 1.95 (s, 3H); 1.69-1.59 (m, 2H); 1.38-1.29 (m, 4H); 0.95-0.85 (m, 3H)

### Example 12C

### 5-Butoxy-4-(2-methylprop-1-en-1-yl)furan-2(5H)-one

Distilled at 0.05 mbar vacuum with by 136-146° C.; GC/MS: m/z=210; ¹H-NMR (400 MHz, CDCl₃) δ 5.94 (s, 1H); 5.86 (s, 1H); 5.80 (s, 1H); 3.85-3.76 (m, 1H); 3.69-3.60 (m, 1H); 2.00 (s, 3H); 1.95 (s, 3H); 1.68-1.58 (m, 2H); 1.45-1.32 (m, 2H); 0.93 (t, 3H, J=7.4 Hz)

### Example 13

### Preparation of 5-methoxy-4-(2-methylpropyl)-dihydrofuran-2(3H)-one

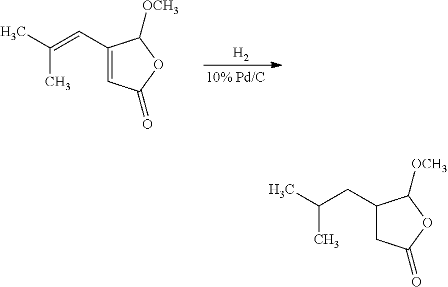

A solution of 5-methoxy-4-(2-methylprop-1-en-1-yl)furan-2(5H)-one (100 g, see example 12) in MTBE (700 mL), 5 g (5 wt %) of 10° A Pd/C was charged to a hydrogenation vessel. 1 atm of hydrogen gas was introduced and pressure was kept constant during the reaction. After 7 h at 20° C., the reaction was filtered through a celite pad (060 mm, H=30 mm) and rinsed with MTBE (3×50 mL). The filtrate was washed with 1M NaHCO₃ solution (200 mL), water, brine and dried on Na₂SO₄. After solvent removal 5-methoxy-4-(2-methylpropyl)-dihydrofuran-2(3H)-one was obtained as colourless liquid (98 g, 96%).

GC/MS: m/z=172

¹H NMR (400 MHz, CDCl₃) δ 5.25 (d, J=5.0 Hz, 0.53H, major isomer), 5.04 (d, J=2.4 Hz, 0.26H, minor isomer), 3.48 (s, 0.92H, minor isomer), 3.46 (s, 1.66H, major isomer), 2.78 (dd, J=17.7, 8.6 Hz, 0.33H), 2.59-2.42 (m, 1.27H, major isomer), 2.42-2.34 (m, 0.33H, minor isomer), 2.29 (dd, J=16.7, 11.8 Hz, 0.63H, major isomer), 2.15 (dd, J=17.7, 4.6 Hz, 0.33H, minor isomer), 1.67-1.31 (m, 2.63H both isomers), 1.28-1.19 (m, 0.33H, minor isomer), 0.95-0.86 (m, 6H both isomers). ¹³C NMR (CDCl₃): 177.7 (C, major isomer), 176.04 (C, minor isomer), 110.1 (CH, minor isomer), 106.1 (CH, major isomer), 57.0 (CH₃, minor isomer), 56.6 (CH₃, major isomer), 41.2 (CH₂, minor isomer), 39.1 (CH₂, minor isomer), 38.3 (CH₂, major isomer), 37.1 (CH₂, major isomer), 33.9 (CH, minor isomer), 32.8 (CH, major isomer), 26.0 (CH, major isomer), 25.8 (CH, minor isomer), 23.0 (CH₃, major isomer), 22.9 (minor isomer), 22.7 (major isomer), 22.6 (minor isomer).

Using the same general method and starting from the compounds of examples 12A, 12B and 12C respectively, the following compounds were also obtained:

### Example 13A

### 5-(3-Methylbutoxy)-4-(2-methylpropyl)-dihydrofuran-2(3H)-one

GC/MS: m/z=228; ¹H-NMR (400 MHz, CDCl₃) δ 5.35 (0.86H, d, J=5.0 Hz) 5.13 (0.14H, d, J=2.7 Hz) 3.87-3.79 (m, 1H) 3.57-3.45 (m, 1H) 2.58-2.37 (2H, m) 2.35-2.27 (0.79H, m) 2.20-2.11 (0.21H, m) 1.74-1.62 (1H, m) 1.61-1.44 (4H, m) 1.42-1.31 (1H, m) 0.95-0.86 (12H, m)

### Example 13B

### 4-(2-Methylpropyl)-5-pentyloxydihydrofuran-2(3H)-one

GC/MS: m/z=228; ¹H-NMR (400 MHz, CDCl₃) δ 5.35 (0.77H, d, J=5.0 Hz) 5.13 (0.23H, d, J=2.6 Hz,) 3.83-3.75 (1H, m) 3.55-3.40 (1H, m) 2.59-2.37 (2H, m) 2.32 (0.73H, dd, J=16.6, 11.8 Hz) 2.15 (0.27H, dd, J=17.7, 5.0 Hz) 1.67-1.45 (4H, m) 1.41-1.25 (5H, m) 0.96-0.86 (9H, m)

### Example 13C

5-Butoxy-4-(2-methylpropyl)-dihydrofuran-2(3H)-one GC/MS: m/z=214; ¹H-NMR (400 MHz, CDCl₃) δ 5.35 (0.8H, d, J=5.0 Hz) 5.13 (0.2H, d, J=2.6 Hz,) 3.85-3.75 (1H, m) 3.56-3.41 (1H, m) 2.59-2.38 (2H, m) 2.31 (0.76H, dd, J=16.5, 11.7 Hz) 2.15 (0.24H, dd, J=17.6, 5.0 Hz) 1.66-1.45 (4H, m) 1.43-1.30 (3H, m) 0.95-0.87 (9H, m)

### Example 14

### Preparation and hydrogenation of 5-(L-Menthyloxy)-4-(2-methyl-1-propenyl)-2(5H)-furanone

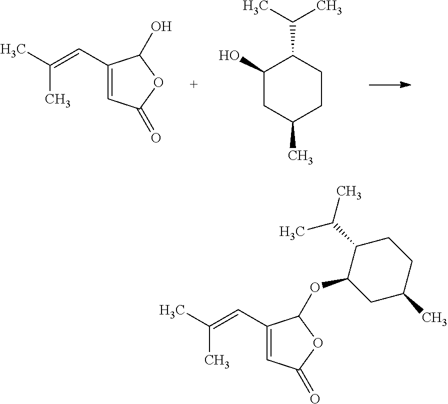 [SMILES: CC(C)=CC1=CC(=O)OC1O ; CC(C)[C@@H]1CC[C@@H](C)C[C@H]1O ; CC(C)=CC1=CC(=O)OC1O[C@@H]1C[C@H](C)CC[C@H]1C(C)C]

A mixture of 5-hydroxy-4-(2-methyl-1-propenyl)-2-furanone (59.5 g, 0.386 mol), L-menthol (89.5 g, 1.5 equivalents) and methanesulfonic acid (1.5 g) was stirred at 70-80° C. under vacuum (to remove water) for 100 h. The liquid mass was poured (hot) into acetonitrile (450 mL) and the title compound isolated by cooling, filtration and washing. The yield was 76.2 g (67%). Suitable crystals for XRD were grown by slow evaporation of an acetone solution. The configuration at the acetal carbon was (R).

Hydrogenation of 5-(L-menthyloxy)-4-(2-methyl-1-propenyl)-2-furanone in ethyl acetate using the conditions of Example 13, gave the saturated derivative in quantitative yield. By ¹H NMR, the compound was a 1:1 mixture of diastereomers.

### Example 15

### Preparation of 5-acetoxy-4-(2-methylprop-1-en-1-yl)furan-2(5H)-one

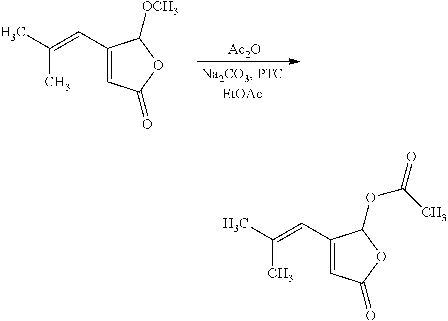

A suspension of 5-hydroxy-4-(2-methylprop-1-en-1-yl)furan-2(5H)-one (19 g, 0.123 mol) in ethyl acetate (100 mL) was treated with solid sodium carbonate (13.1 g, 0.123 mol) and tetrabutylammonium hydrogensulfate (0.5 g). Acetic anhydride (18.8 g, 1.5 eq.) was added in one portion. A mildly exothermic reaction ensued. The mixture was stirred overnight, water (100 mL) added and the organic phase separated (pH of aqueous phase was 6.0). The ethyl acetate phase was water washed and concentrated to leave a solid (24.6 g, 100%). This was pure by TLC (100:3 CPME:acetic acid). If this reaction is carried out in isopropyl acetate, the pure compound can be isolated by cooling the isopropyl acetate solution after the water washing in about 70% yield.

m/z: 196

### Example 16

### Hydrogenation of 5-acetoxy-4-(2-methylprop-1-en-1-yl)furan-2(5H)-one

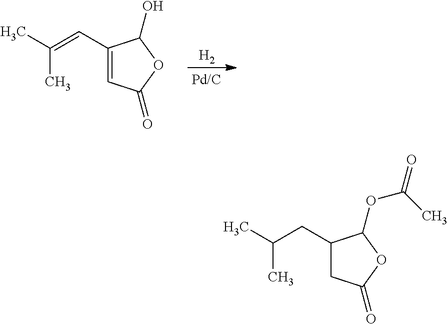

The material of example 34 was hydrogenated under similar conditions to those outlined in Example 13 to give a >90% yield of 5-acetoxy-4-(2-methylpropyl)-3,4-dihydro-2(5H)-furanone as a 1:1 mixture of diastereomers. The product is accompanied by ca. 5% of 4-(2-methylpropyl)-3,4-dihydrofuran-2(5H)-one. The product can be hydrolysed as in Example 30 to give yields of 5-hydroxy-4-(2-methylpropyl)-3,4-dihydro-5H-2-furanone/3-formyl-5-methylhexanoic acid.

### Example 17

### Preparation of 5-hydroxy-4-(2-methylpropyl)dihydrofuran-2(3H)-one by hydrogenation of 5-hydroxy-4-(2-methylprop-1-en-1-yl)furan-2(5H)-one in alkaline solution

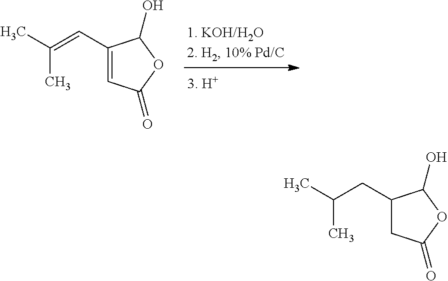

5-Hydroxy-4-(2-methylpropen-1-yl)-2-furanone (3.35 g, 21.7 mmol) and water (20 mL) were charged to a hydrogenator. Potassium hydroxide (1.21 g, 1 eq) was then charged to the vessel and the contents were heated to 40° C. until dissolution had occurred. 10% Pd/carbon catalyst (0.67 g) was charged to the vessel and the reactor contents were then hydrogenated at 40° C. and 5 barg hydrogen pressure. On completion of reaction the reaction was cooled and the catalyst removed by filtration. The pH was adjusted to pH 2 by addition of 36% hydrochloric acid and the aqueous layer was washed with toluene to extract the desired product. The combined toluene extracts were concentrated to give the title compound (3.17 g, 92%).

### Example 18

### Hydrogenation of 5-hydroxy-4-(2-methylprop-1-en-1-yl)furan-2(5H)-one in neutral solution

5-Hydroxy-4-(2-methylpropen-1-yl)-2-furanone was hydrogenated in 6 mL of water per gram starting material with 10% w/w of Pd/C catalyst. (22 h, 40° C., 10 bar). On filtration, an oily phase separated from the water. This was a 1:1 mixture of 5-hydroxy-4-(2-methylpropyl)dihydrofuran-2(3H)-one (I^{A}) and 4-(2-methylpropyl)-dihydrofuran-2-one. Compound (I^{A}) can easily be separated pure by an acid base extraction. This reaction was repeated in organic solvents with 2-propanol giving relatively pure (I^{A}).

### Example 19

### Preparation of 5-hydroxy-4-(2-methylpropyl)dihydrofuran-2(3H)-one (I^{A}) from 5,5′-(piperazine-1,4-diyl)bis(4-(2-methylprop-1-en-1-yl)furan-2(5H)-one) in one pot

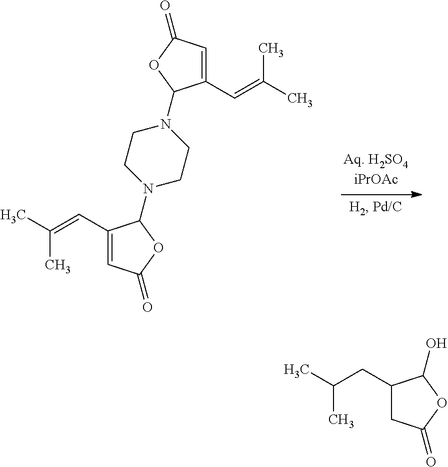

5,5′-(Piperazine-1,4-diyl)bis(4-(2-methylprop-1-en-1-yl)furan-2(5H)-one) (50.0 g, 0.14 mol) was charged to water (300 mL) containing sulphuric acid (16.0 g, 0.163 mol). Isopropyl acetate (200 mL) was added. 50% water wet 5% palladium on carbon (3.0 g) was added and the mixture was hydrogenated (5 bar hydrogen) at ambient temperature for 15 h. The reaction mixture was filtered from catalyst, washed with isopropyl acetate (300 mL)—this was also used to wash out the vessel. The organic phase was separated, washed with water (100 mL) and concentrated on a rotary evaporator to afford 38.8 g of clear oil. This was a mixture of desired compound (I^{A}) and the over-reduced lactone (4-(2-methylpropyl)-dihydrofuran-2-one). It was readily purified by extraction with aqueous potassium carbonate solution and wash with toluene. Acidification of the potassium carbonate extract with formic acid gave the desired product (I^{A}) (25.6 g, 58%).

¹H NMR (D₂O with added K₂CO₃): δ 9.5 (1H), 2.8 (m, 2H), 2.40 (m, 2H), 1.55 (m, 2H), 1.30 (m, 2H), 0.95 (m, 6H).

### Example 20

### Preparation of 3-isobutylidene-2-oxopentanedioic acid mono-potassium salt

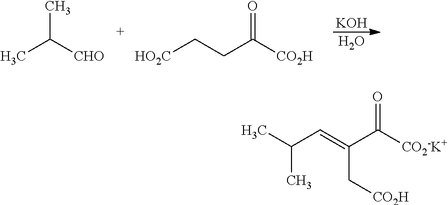

A 2 L round bottom was charged 300 g α-ketoglutaric acid and 450 mL of ice water. With stirring and cooling 314 g of potassium hydroxide in 450 mL water was charged keeping the pot temperature less than 25° C. Reactor was placed under nitrogen and isobutyraldehyde was added at 0.2 mL/min over 50 hrs. The resulting two phase yellow solution was separated and washed with 100 mL MTBE. The lower aqueous phase pH was adjusted to 3.2-3.4 and was concentrated to ⅓ volume at 65° C. under vacuum. After cooling to <5° C. the resulting solids were collected and washed with several small volumes of water to give after drying 230 g of a white solid containing 60% 3-isobutylidene-2-oxopentanedioic acid mono-potassium salt and remainder KCl.

¹H NMR (D₂O at pH 8-10, 400 MHz) δ 6.4 (d, 1H), 3.3 (s, 2H), 2.7 (m, 1H), 0.96 (d, 6H).

¹³C NMR (D₂O at pH 8-10, 400 MHz) δ 23 (2CH₃), 31 (CH), 35 (CH₂), 133 (C), 164 (CH), 170 (C), 176 (C), 182 (C).

### Example 21

### Preparation of 3-(2-methylpropyl)-2-oxopentanedioic acid mono-potassium salt

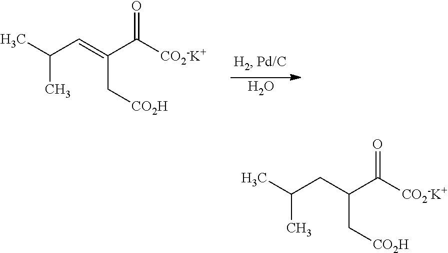

An aqueous solution of 3-isobutylidene-2-oxopentanedioic acid mono-potassium salt as generated in example 20 was adjusted to pH 6-8 and 5% Pd/C was added and the solution was hydrogenated at 10 bar H₂ 25° C. for 10 hours. The catalyst was removed by filtration and the pH of the solution adjusted to pH 3.8. The mixture was cooled to <5° C. and filtered and washed with a small amount of cold water. The resulting white crystals were dried at 40° C. under vacuum to give the title compound, 286 g, 58% yield over 2 steps.

¹H NMR (D₂O at pH 8-10, 400 MHz) δ 3.5 (m, 1H), 2.5 (dd, 1H), 2.3 (dd, 1H), 1.6-1.5 (m, 2H) 1.3-1.2 (m, 1H) 0.9 (d, 6H). ¹³C NMR (D₂O at pH 8-10, 400 MHz) δ 21.7 (CH₃), 22.2 (CH₃), 25.5 (CH), 38.4 (CH₂), 39.3 (CH₂), 43.4 (CH), 167.8 (C), 170.6 (C), 180.7 (C).

### Example 22

### Expression of Decarboxylase Enzymes in E. coli

PubMed was used to search the literature for decarboxylase enzymes with a broad spectrum of activity. The KEGG (Kyoto Encyclopedia of Genes and Genomes) program was used to search for microbial decarboxylases with activity on compounds somewhat similar in structure to 3-(2-methylpropyl)-2-oxopentanedioic acid or 3-isobutylidene-2-oxopentanedioic acid. Seven classes of decarboxylases were chosen for investigation based on reports of activity on compounds with some structural similarity. The decarboxylase classes were glutamate decarboxylase, diaminopimelate decarboxylase, indolepyruvate decarboxylase, branched-chain α-keto acid decarboxylase, aromatic-L-amino-acid decarboxylase, lysine decarboxylases and benzoylformate decarboxylase. Forty one sequences of genes for proven, or putative decarboxylase enzymes were selected. The genes were codon optimized for expression in E. coli, synthesized by GeneArt (Germany), DNA2.0 (Menlo Park, Calif. USA), or Blue Heron Biotechnology (Bothell, Wash. USA), cloned into expression vector pSTRC52 (Pfizer Inc., USA), and put into the expression strain E. coli BDG62 (Pfizer Inc., USA) (Table 1 below). Two genes were amplified from genomic DNA from the appropriate organisms by PCR and cloned into the same expression vector and E. coli strain. Four decarboxylase enzymes were purchased from Sigma and tested for activity on 3-(2-methylpropyl)-2-oxopentanedioic acid.

Each E. coli strain containing the cloned decarboxylase gene was grown overnight in LB broth with the appropriate antibiotic. A small amount (50 μL-100 μL) of the overnight seed culture was used to inoculate 4.0 mL of Terrific Broth medium with appropriate antibiotics in 20 mm diameter culture tubes. The cultures were grown in a shaking incubator at 32° C. and 300 rpm. After 5 h of growth IPTG was added to 0.4 mM final concentration to induce the enzyme expression. The cultures were returned to the incubator and grown for an additional 19 h.

### Example 23

### Decarboxylation of 3-(2-methylpropyl)-2-oxopentanedioic acid potassium salt and 3-isobutylidene-2-oxopentanedioic acid potassium salt with Decarboxylase Enzymes

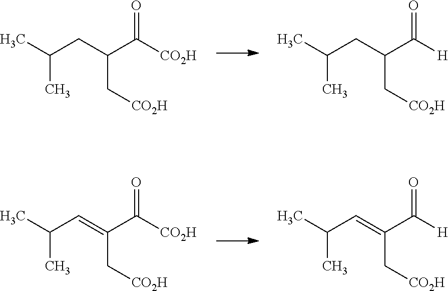

Recombinant decarboxylase enzymes were tested for decarboxylation of 3-(2-methylpropyl)-2-oxopentanedioic acid (XV) and 3-isobutylidene-2-oxopentanedioic acid (XII) using E. coli cells prepared as described in Example 22. Reactions (1 mL) were carried out at 37° C. in potassium phosphate buffer (100 mM, pH 6.4) with decarboxylase (60 mg wet cells), 50 mM 3-(2-methylpropyl)-2-oxopentanedioic acid or 3-isobutylidene-2-oxopentanedioic acid, ThDP (0.1 mM), and MgSO₄ (2.5 mM). Decarboxylation of 3-(2-methylpropyl)-2-oxopentanedioic acid and 3-isobutylidene-2-oxopentanedioic acid was determined by a UPLC assay. An aliquot (0.1 mL) of the reaction was treated with 0.5 mL of potassium phosphate buffer (100 mM, adjusted to pH 2.2 with phosphoric acid) and 0.3 mL of a solution of 2,4-dinitrophenylhydrazine (20 mM in 1M HCl:acetonitrile, 3:1, v/v) for 30 min at 50° C. The derivatized samples were diluted with 0.5 mL of acetonitrile, filtered and analyzed by UPLC on a Agilent Eclipse Plus C18 column (100 mm×3.0 mm, 1.8 μm) eluted with 0.1% trifluoroacetic acid in water:acetonitrile (55:45, v/v) at 1.1 mL/min. The column was maintained at 40° C. and the effluent was monitored at 360 nm and ES⁺ mass spectroscopy. Positive results were indicated by the presence of a peak for 3-formyl-5-methylhexanoic acid from 3-(2-methylpropyl)-2-oxopentanedioic acid or 3-formyl-5-methylhex-3-enoic acid from 3-isobutylidene-2-oxopentanedioic acid. Results of the analyses are shown in Table 1.

TABLE 1

| | | | |
|---|---|---|---|
| Enzyme | | Reaction | Reaction |
| Name | Source Organism | on (XV) | on (XII) |

Glutamate decarboxylases

| | | | |
|---|---|---|---|
| Aa gad | Arthrobacter aurescens | −^{a} | NT^{b} |
| Rs gad | Rhodococcus sp. RHA1 | − | NT |
| Sc gad | Streptomyces coelicolor | − | NT |
| Dz gad | Dickeya zeae | − | NT |
| Pa gad | Pyrobaculum arsenaticum | − | NT |
| Mm gad | Mycobacterium marinum | − | NT |
| UT gad | UTI89 metagenomic DNA | − | NT |
| Rm gad | Ralstonia metaffidurans | − | NT |
| Re gad | Ralstonia eutropha | − | NT |
| Xn gad | Xenorhabdus nematophila | − | NT |
| Pd gad | Photobacterium damselae | − | NT |

Branched-chain α-Keto Acid decarboxylase

| | | | |
|---|---|---|---|
| Pa bkd | Photorhabdus asymbiotica | − | − |
| Sv bkdA | Streptomyces virginiae | − | − |
| Ll kdcA | Lactococcus lactis | + | + |

Indolepyruvate decarboxylases

| | | | |
|---|---|---|---|
| Ss ipd | Staphylococcus saprophyticus | − | + |
| Dd ipd | Desulfovibrio desulfuricans | + | + |
| Bm ipd | Bacillus megaterium | − | − |
| Dv ipd | Desulfovibrio vulgaris | − | − |
| Aa ipd | Aromatoleum aromaticum | − | − |
| Ab ipd | Azospirillum brasilense | − | − |
| Bs ipd | Bradyrhizobium sp. BTAi1 | − | − |
| NC ipd | NC10 bacterium | − | − |
| Rr ipd | Rhodospirillum rubrum | − | − |

Aromatic-L-amino-acid decarboxylases

| | | | |
|---|---|---|---|
| Ab dcd | Acinetobacter baumannii | − | − |
| Bp dcd | Bacillus pumilus | − | − |
| Cg dcd | Chryseobacterium gleum | − | − |
| As dcd | Anaeromyxobacter sp. Fw109-5 | − | − |
| Bs dcd | Burkholderia sp. 383 | + | − |
| Cs dcd | Cyanothece sp. PCC 8801 | − | − |

Lysine decarboxylases

| | | | |
|---|---|---|---|
| Cr lysA | Candidatus Carsonella ruddii | − | − |
| Dt lysA | Dictyoglomus thermophilum | − | − |
| Sa lysA | Sphingopyxis alaskensis | − | − |

Benzoylformate decarboxylases

| | | | |
|---|---|---|---|
| Rp bfd | Rhodopseudomonas palustris | + | + |
| Sc bfd | Streptomyces coelicolor | + | + |
| Rc bfd | Ricinus communis | − | − |
| Pp bfd | Pseudomonas putida | + | + |
| Ms bfd | Mycobacterium smegmatis | − | − |
| As bfd | Arthrobacter sp. FB24 | + | + |
| Pb bfd | Pseudomonas brassicacearum | + | + |
| Ct bfd | Comamonas testosterone | + | + |
| Dd bfd | Desulfovibrio desulfuricans | + | + |
| Rj bfd | Rhodococcus jostii | + | + |
| Pf bfd | Pseudomonas fluorescens | + | + |

Purified Enzymes (purchased from Sigma-Aldrich)

| | | | |
|---|---|---|---|
| L-Histidine | Lactobacillus 30a | − | NT |
| Decarboxylase | | | |
| L-Lysine | Bacterium cadaveris | − | NT |
| Decarboxylase | | | |
| L-Tyrosine | Streptococcus faecalis | − | NT |
| Decarboxylase | | | |
| Pyruvate | Saccharomyces cerevisiae | − | NT |
| Decarboxylase | | | |

^{a}−: No detection of appropriate product
^{b}NT: Not tested
^{c}+: Detection of appropriate product

### Example 24

### Preparation of 3-Formyl-5-methylhex-3-enoic acid

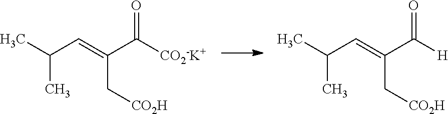

In a 250 mL round bottom flask 80 g of cell concentrate comprising E. Coli expressing Pseudomonas putida benzoylformate decarboxylase (see example 23) was charged. To this was added a pH 6.2 adjusted solution of 3-isobutylidene-2-oxopentanedioic acid mono-potassium salt (10 g, see example 20) in 50 mL water, with 0.5 g of magnesium sulfate and 0.5 g of thiamine pyrophosphate. The resulting pH 6.2 slurry was heated to 50° C. and stirred for 145 hrs keeping the pH adjusted between 6.2 and 7.2 with concentrated HCl. The reaction mixture was cooled to RT and centrifuged. The aqueous decants were washed with 50 mL MTBE with minimum agitation. The pH of the aqueous phase was adjusted to 4 and filtered through celite. The filtrate was extracted with minimum agitation three times with 50 mL MTBE. The MTBE was dried over anhydrous sodium sulfate and concentrated to a thick red oil. This oil was extracted with several portions of hot hexanes; the combined hexanes were cooled to <0° and the resulting crystals were isolated and dried in air to give 3-formyl-5-methylhex-3-enoic acid as a white solid (0.6 g).

¹H NMR (D₂O at pH 8-10, 400 MHz) δ 9.2 (s, 1H), 6.6 (d, 1H), 3.1 (s, 2H), 2.7 (m, 1H), 1 (d, 6H)

### Example 25

### Expression of Enoate Reductase Homologues in E. coli

The DNA sequence corresponding to the gene for Lycopersicon esculentum (tomato) 12-oxophytodienoate reductase 1 (OPR1) was retrieved from the Genbank database (accession number AC Q9XG54) and was synthesized by GeneArt (Germany). The sequence was codon optimized for expression in E. coli, and was subcloned into an E. coli expression plasmid pSTRC18 (Pfizer Inc., USA). The protein sequence is shown below. The OPR1 expression construct was transformed into BL21(DE3) E. coli (Stratagene, Agilent Technologies, Santa Clara, Calif., USA) as directed and overnight cultures were incubated in LB+streptomycin media. The LB culture was used to inoculate expression cultures (LB, M9Y, or TB), which were incubated at 37° C. (210 rpm) After the culture reached a suitable biomass concentration (OD 1 at A600), IPTG was added (1 mM) and cultures were incubated for another 20 h (30° C., 210 rpm). The cells were harvested by centrifugation (4000×g, 30 min, 4° C.) and stored at −20° C.

The BLASTP program was used to search the NCBI non-redundant protein sequences database for gene sequences related to 12-Oxophytodienoate reductase (OPR1) from Lycopersicon esculentum. Thirty eight sequences for related genes were selected, codon optimized for expression in E. coli, and subcloned into the pET28b(+) E. coli expression plasmid (Novagen, EMD Chemicals, Gibbstown, N.J., USA). The OPR1 related expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C.

Lycopersicon esculentum (tomato) 12-Oxophytodienoate Reductase 1 protein sequence:

(SEQ ID NO. 8)

MENKWEEKQ VDKIPLMSPC KMGKFELCHR WLAPLTRQR
SYGYIPQPHA ILHYSQRSTN GGLLIGEATV ISETGIGYKD
VPGIWTKEQV EAWKPIVDAV HAKGGIFFCQ IWHVGRVSNK
DFQPNGEDPI SCTDRGLTPQ IRSNGIDIAH FTRPRRLTTD
EIPQIVNEFR VAARNAIEAG FDGVEIHGAH GYLIDQFMKD
QVNDRSDKYG GSLENRCRFA LEIVEAVANE IGSDRVGIRI
SPFAHYNEAG DTNPTALGLY MVESLNKYDL AYCHWEPRM
KTAWEKIECT ESLVPMRKAY KGTFIVAGGY DREDGNRALI
EDRADLVAYG RLFISNPDLP KRFELNAPLN KYNRDTFYTS
DPIVGYTDYP FLETMT

Lycopersicon esculentum (tomato) 12-Oxophytodienoate Reductase 1 codon optimized sequence:

(SEQ ID NO. 9)

ATGGAAAACAAAGTTGTGGAAGAAAAACAGGTTGATAAAATCCCGCTGAT
GAGCCCGTGTAAAATGGGTAAATTCGAGCTGTGTCATCGCGTTGTACTGG
CACCGCTGACTCGTCAGCGTTCTTATGGTTACATTCCGCAGCCGCACGCA
ATCCTGCATTACTCTCAGCGCAGCACCAACGGTGGCCTGCTGATCGGTGA
AGCAACCGTGATCAGCGAAACTGGCATCGGTTACAAAGATGTGCCGGGTA
TCTGGACGAAAGAGCAGGTTGAGGCCTGGAAACCGATCGTCGACGCGGTG
CATGCCAAAGGTGGTATTTTCTTTTGTCAGATCTGGCACGTTGGTCGTGT
ATCCAACAAAGATTTTCAGCCGAACGGCGAAGATCCGATTTCCTGTACTG
ACCGCGGCCTGACCCCGCAGATCCGTTCCAACGGCATTGACATTGCCCAC
TTCACCCGTCCACGTCGCCTGACTACTGACGAGATTCCGCAGATCGTGAA
CGAGTTCCGCGTTGCAGCGCGTAATGCTATTGAAGCGGGTTTCGATGGCG
TCGAGATTCATGGTGCCCACGGTTACCTGATCGACCAATTCATGAAAGAC
CAAGTTAACGACCGCAGCGATAAGTATGGCGGTTCTCTGGAGAACCGTTG
TCGCTTCGCGCTGGAAATCGTTGAAGCAGTAGCCAACGAGATTGGCTCCG
ACCGTGTTGGTATCCGTATCTCTCCATTCGCACACTACAACGAAGCGGGC
GACACTAACCCGACCGCACTGGGCCTGTATATGGTGGAGAGCCTGAATAA
ATACGACCTGGCGTATTGTCACGTGGTCGAGCCGCGCATGAAAACCGCCT
GGGAAAAGATTGAGTGCACCGAAAGCCTGGTGCCGATGCGTAAAGCCTAC
AAAGGCACCTTCATCGTAGCTGGTGGCTACGACCGTGAAGACGGTAACCG
CGCTCTGATCGAAGACCGTGCCGACCTGGTTGCGTACGGTCGTCTGTTCA
TCAGCAACCCAGACCTGCCGAAGCGTTTTGAACTGAACGCTCCGCTGAAC
AAATACAACCGTGACACTTTCTACACTTCCGACCCGATCGTTGGTTACAC
CGATTACCCGTTTCTGGAAACTATGACTTAATAA

### Example 26

### Reduction of (E)-3-formyl-5-methylhex-3-enoic acid with Recombinant Reductases

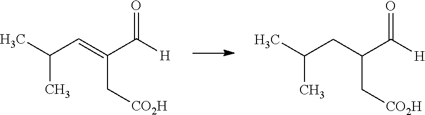

Recombinant enoate reductases were tested for reduction of (E)-3-formyl-5-methylhex-3-enoic acid using E. coli cells prepared as described in Example 25. Reactions (0.5 mL) were carried out at 30° C. in potassium phosphate buffer (100 mM, pH 7.0) with E. coli cells (100 mg wet cells/mL), NADPH (10 mM), NADH (10 mM) and (E)-3-formyl-5-methylhex-2-enoic acid (10 mM). After 16 h, acetonitrile (0.5 ml) was added to each reaction and the resulting mixtures were centrifuged (2000 rpm×5 min). Aliquots (0.1 mL) of the resulting supernatants were treated with 0.1 mL of potassium phosphate buffer (100 mM, adjusted to pH 2.2 with phosphoric acid) and 0.225 mL of a solution of 2,4-dinitrophenylhydrazine (20 mM in 1M HCl:acetonitrile, 3:1, v/v) for 30 min at 50° C. The derivatized samples were diluted with 0.225 mL of acetonitrile and analyzed by HPLC on a Phenomenex Lux 5μ Amylose-2 column (250 mm×4.6 mm id) eluted with 0.1% trifluoroacetic acid in water:acetonitrile (65:35, v/v) at 2 mL/min. The column was maintained at 50° C. and the effluent was monitored at 360 nm. Results of HPLC analyses are shown in Table 2.

TABLE 2

| | | Accession | % |
|---|---|---|---|
| Entry | Enzyme Name/Source | Number | Conversion¹ |
| 1 | Old yellow enzyme 1/ | Q02899 | 6.5 |
| | Saccharomyces carlsbergensis | | |
| 2 | Old yellow enzyme 2/ | Q03558 | 9.2 |
| | Saccharomyces cerevisiae | | |
| 3 | Old yellow enzyme 3/ | P41816 | 8.6 |
| | Saccharomyces cerevisiae | | |
| 4 | NADH:flavin oxidoreductase/ | Q5NLA1 | 85.0 |
| | Zymomonas mobilis | | |
| 5 | fumarate reductase/Shewanella | Q07WU7 | 3.4 |
| | frigidimarina | | |
| 6 | Pentaerythritol tetranitrate | Q6JL81 | 85.6 |
| | reductase/Enterobacter cloacae | | |
| 7 | Unnamed enzyme/Arabidopsis | BAH57049 | 3.7 |
| | thaliana | | |
| 8 | Allyl alcohol dehydrogenase/ | BAA89423 | 5.0 |
| | Nicotiana tabacum | | |
| 9 | Artemisinic aldehyde delta-11(13) | 1WLY_A | 37.1 |
| | reductase/Artemisia annua | | |
| 10 | 2-haloacrylate reductase/ | NP_390263 | 4.2 |
| | Burkholderia sp. WS | | |
| 11 | NADPH dehydrogenase/Bacillus | YP_390263 | 82.8 |
| | subtilis subsp. subtilis str. 168 | | |
| 12 | NADH:flavin | YP_001664021 | 54.9 |
| | oxidoreductase/NADH oxidase/ | | |
| | Thermoanaerobacter | | |
| | pseudoethanolicus | | |
| 13 | Unnamed enzyme/Clostridium | CAA71086 | 6.0 |
| | tyrobutyricum | | |
| 14 | Unnamed enzyme/Moorella | Q2RGT7 | 4.8 |
| | thermoautotrophica | | |
| 15 | oxophytodienoate reductase | Q9XG54 | 31.4 |
| | (OPR1)/Lycopersicon esculentum | | |
| 16 | Oxophytodienoate reductase | Q9FEW9 | 15.7 |
| | (OPR3)/Lycopersicon esculentum | | |
| 17 | N-ethylmaleimide reductase/ | Q3Z206 | 100.0 |
| | Shigella sonnei | | |
| 18 | 12-oxo-phytodienoic acid | Q49HE0 | 100.0 |
| | reductase/Zea mays | | |
| 19 | 12-oxo-phytodienoic acid | Q49HE4 | 100.0 |
| | reductase/Zea mays | | |
| 20 | Unnamed enzyme/Vitis vinifera | A5BF80 | 7.5 |
| 21 | Unnamed enzyme/Populus | B9MWG6 | 100.0 |
| | trichocarpa | | |
| 22 | 12-oxophytodienoate reductase/ | Q8GYB8 | 100.0 |
| | Arabidopsis thaliana | | |
| 23 | 12-oxophytodienoate reductase/ | B9SK95 | 5.4 |
| | Castor bean | | |
| 24 | NADH:flavin | D0YIM0 | 100.0 |
| | oxidoreductase/NADH oxidase/ | | |
| | Klebsiella variicola | | |
| 25 | Unnamed enzyme/Citrobacter | A8AH31 | 100.0 |
| | koseri | | |
| 26 | N-ethylmaleimide reductase/ | C1M473 | 100.0 |
| | Citrobacter sp. | | |
| 27 | N-ethylmaleimide reductase/ | D2THI8 | 100.0 |
| | Citrobacter rodentium | | |
| 28 | N-ethylmaleimide reductase/ | Q5PH09 | 100.0 |
| | Salmonella paratyphi | | |
| 29 | N-ethylmaleimide reductase/ | C9Y3L1 | 100.0 |
| | Cronobacter turicensis | | |
| 30 | Unnamed enzyme/Providencia | B2Q290 | 100.0 |
| | stuartii | | |
| 31 | NADPH dehydrogenase/Yarrowia | Q6CI57 | 9.1 |
| | lipolytica | | |
| 32 | N-ethylmaleimide reductase/ | Q88129 | 17.4 |
| | Pseudomonas putida | | |
| 33 | 12-oxophytodienoate reductase/ | 150864790 | 7.8 |
| | Pichia stipitis | | |
| 34 | NAPDH dehydrogenase/Pichia | 126131638 | 12.6 |
| | stipitis | | |
| 35 | Unnamed enzyme/Pichia | 146393506 | 21.6 |
| | guiffiermondii | | |
| 36 | unnamed enzyme/Candida | 50293551 | 36.5 |
| | glabrata | | |
| 37 | Unnamed enzyme/ | 50405397 | 9.1 |
| | Debaryomyces hansenii | | |
| 38 | Unnamed enzyme/Geobacillus | Q5KXG9 | 100.0 |
| | kaustophilus HTA426 | | |
| 39 | Oxophytodienoate reductase | Q9XG54 | 14.0 |
| | (OPR2)/Lycopersicon | | |
| | esculentium | | |

¹% Conversion of (E)-3-formyl-5-methylhex-2-enoic acid to 3-formyl-5-methylhexanoic acid

### Example 27

### Reduction of (E)-3-formyl-5-methylhex-3-enoic acid with Recombinant Reductases and Formate Dehydrogenase

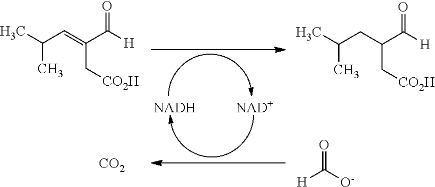

Recombinant enoate reductases were evaluated for reduction of (E)-3-formyl-5-methylhex-3-enoic acid with NAD⁺ and formate dehydrogenase. Enoate reductases were expressed in E. coli cells as described in Example 25. Formate dehydrogenase was expressed in E. coli cells as follows: The pET26b formate dehydrogenase expression construct was transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB Medium+kanamycin (Novagen, EMD Chemicals, Gibbstown, N.J., USA). Cultures were incubated for 20 h at 30° C., and the cells were harvested by centrifugation (4000×g, 30 min, 4° C.) and stored at −20° C. A variant (D74M) of oxophytodienoate reductase 3 (Accession number Q9FEW9) was expressed in E. coli cells as follows: QuikChange Site-directed Mutagenesis kit from Stratagene (La Jolla, Calif., USA) was used to create oxophytodienoate reductase 3 variant D74M as directed. Primers were ordered from Integrated DNA Technologies (Coralville, Iowa, USA). The pSTRC18 oxophytodienoate reductase 3 (OPR3) expression construct was transformed into BL21(DE3) E. coli (Stratagene, Agilent Technologies, Santa Clara, Calif., USA) as directed and overnight cultures were grown in expansion broth+streptomycin (Zymo Research, Irvine, Calif., USA). The LB cultures were used to inoculate expression cultures grown in Overexpression broth+streptomycin (Zymo Research, Irvine, Calif., USA). Cultures were incubated for 20 h at 23° C., and the cells were harvested by centrifugation (4000×g, 30 min, 4° C.) and stored at −20° C.

Reactions (0.5 mL) were carried out at 30° C. in potassium phosphate buffer (100 mM, pH 7.0) with enoate reductases (40 mg wet cells/mL), formate dehydrogenase (80 mg wet cells/mL), NAD⁺ (0.02 mM), ammonium formate (30 mM) and (E)-3-formyl-5-methylhex-3-enoic acid (20 mM). After 24 h, reaction mixtures were acidified with 0.025 mL of 4N HCl and extracted with 1 mL of ethyl acetate. Aliquots (0.5 mL) of the ethyl acetate extracts (0.5 mL) were dried over anhydrous sodium sulfate and treated with methanol (0.02 mL) and (trimethylsilyl)diazomethane (0.01 mL of a 2M solution in diethyl ether) to derivatize carboxylic acid moieties to their corresponding methyl esters. The derivatized samples were analyzed by GC on a Chiraldex™ G-TA column (30M×0.25 mm column, column temperature: 135° C. isothermal, injector temperature: 200° C., carrier gas: helium, flow rate approximately 1 mL/min) to give the results shown in Table 3.

TABLE 3

| | | Accession | % |
|---|---|---|---|
| Entry | Enzyme Name/Source | Number | Conversion¹ |
| 1 | NADH:flavin oxidoreductase/ | Q5NLA1 | 23.8 |
| | Zymomonas mobilis | | |
| 2 | Pentaerythritol tetranitrate | Q6JL81 | 19.6 |
| | reductase/Enterobacter cloacae | | |
| 3 | NADPH dehydrogenase/Bacillus | YP_390263 | 17.0 |
| | subtilis subsp. subtilis str. 168 | | |
| 4 | NADH:flavin oxidoreductase/NADH | YP_001664021 | 15.9 |
| | oxidase/Thermoanaerobacter | | |
| | pseudoethanolicus | | |
| 5 | oxophytodienoate reductase (OPR | | 43.5 |
| | 3) variant D74M/Lycopersicon | | |
| | esculentum | | |
| 6 | N-ethylmaleimide reductase/ | Q3Z206 | 24.0 |
| | Shigella sonnei | | |
| 7 | Unnamed enzyme/Populus | B9MWG6 | 31.8 |
| | trichocarpa | | |
| 8 | N-ethylmaleimide reductase/ | C1M473 | 22.8 |
| | Citrobacter sp. 30_2 | | |
| 9 | N-ethylmaleimide reductase/ | C9Y3L1 | 26.3 |
| | Cronobacter turicensis DSM 18703 | | |
| 10 | Unnamed enzyme/Geobacillus | Q5KXG9 | 21.7 |
| | kaustophilus HTA426 | | |
| 11 | 12-oxophytodienoate reductase | Q9XG54 | 39.8 |
| | (OPR1)/Lycopersicon esculentum | | |

¹% Conversion of (E)-3-formyl-5-methylhex-2-enoic acid to 3-formyl-5-methylhexanoic acid (all reactions gave approximately 1:1 mixtures of (S)- and (R)-enantiomers

### Example 28

### Reduction of (E)-3-formyl-5-methylhex-3-enoic acid with Recombinant Reductases

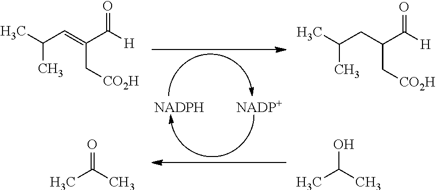

Recombinant enoate reductases were evaluated for reduction of (E)-3-formyl-5-methylhex-3-enoic acid with NADP⁺ and Lactobacillus brevis alcohol dehydrogenase (X-zyme). Enoate reductases were expressed in E. coli cells as described in Example 25. A variant (D74M) of oxophytodienoate reductase 3 (OPR3) was prepared as described in Example 27. Reactions (0.5 mL) were carried out at 30° C. in potassium phosphate buffer (100 mM, pH 7.0) with enoate reductases (40 mg wet cells/mL), Lactobacillus brevis alcohol dehydrogenase (32 U/mL), NADP⁺ (0.02 mM), 2-propanol (3 vol %) and (E)-3-formyl-5-methylhex-2-enoic acid (20 mM). After 24 h, reaction mixtures were acidified with 0.025 mL of 4N HCl and extracted with 1 mL of ethyl acetate. Aliquots (0.5 mL) of the ethyl acetate extracts (0.5 mL) were dried over anhydrous sodium sulfate and treated with methanol (0.02 mL) and (trimethylsilyl)diazomethane (0.01 mL of a 2M solution in diethyl ether) to derivatize carboxylic acid moieties to their corresponding methyl esters. The derivatized samples were analyzed by GC on a Chiraldex™ G-TA column (30M×0.25 mm column, column temperature: 135° C. isothermal, injector temperature: 200° C., carrier gas: helium, flow rate approximately 1 mL/min) to give the results shown in Table 4.

TABLE 4

| | | Accession | % |
|---|---|---|---|
| Entry | Enzyme Name/Source | Number | Conversion¹ |
| 1 | NADH:flavin oxidoreductase/ | Q5NLA1 | 73.0 |
| | Zymomonas mobilis | | |
| 2 | Pentaerythritol tetranitrate reductase/ | Q6JL81 | 100.0 |
| | Enterobacter cloacae | | |
| 3 | NADPH dehydrogenase/Bacillus | YP_390263 | 100.0 |
| | subtilis subsp. subtilis str. 168 | | |
| 4 | NADH:flavin oxidoreductase/NADH | YP_001664021 | 100.0 |
| | oxidase/Thermoanaerobacter | | |
| | pseudoethanolicus | | |
| 5 | oxophytodienoate reductase (OPR 3) | | 96.8 |
| | variant D74M/Lycopersicon | | |
| | esculentum | | |
| 6 | N-ethylmaleimide reductase/Shigella | Q3Z206 | 100.0 |
| | sonnei | | |
| 7 | Unnamed enzyme/Populus | B9MWG6 | 100.0 |
| | trichocarpa | | |
| 8 | N-ethylmaleimide reductase/ | C1M473 | 100.0 |
| | Citrobacter sp. 30_2 | | |
| 9 | N-ethylmaleimide reductase/ | C9Y3L1 | 99.5 |
| | Cronobacter turicensis DSM 18703 | | |
| 10 | Unnamed enzyme/Geobacillus | Q5KXG9 | 100.0 |
| | kaustophilus HTA426 | | |
| 11 | 12-oxophytodienoate reductase | Q9XG54 | 34.3 |
| | (OPR1)/Lycopersicon esculentum | | |

¹%Conversion of (E)-3-formyl-5-methylhex-2-enoic acid to 3-formyl-5-methylhexanoic acid (all reactions gave approximately 1:1 mixtures of (S)- and (R)-enantiomers

### Example 29

### Preparation of 3-formyl-5-methylhexanoic acid

A reaction vessel was charged with 7.5 mL potassium phosphate buffer (0.1M, pH 7.0), 8.4 mg NADP⁺, 0.2 mL Lactobacillus brevis alcohol dehydrogenase (32 U/mL, X-zyme), 0.3 mL 2-propanol, pentaerythritol tetranitrate reductase (2 mL of a 200 mg/mL suspension of E. coli cells in potassium phosphate buffer), and 156 mg of (E)-3-formyl-5-methylhex-3-enoic acid, and agitated at 40° C. After 6.75 h, the reaction mixture was centrifuged and the supernatant was adjusted to pH 2 with 4N HCl and extracted with ethyl acetate (2×10 mL). The ethyl acetate extract was dried over anhydrous magnesium sulfate, filtered and concentrated under reduced pressure to give 141 mg of colorless oil (89% yield).

¹H NMR (D₂O at pH 8-10) δ 2.53-2.43 (m, 2H), 2.34-2.28 (m, 1H), 1.53-1.42 (m, 1H), 1.41-1.34 (m, 1H), 1.20-1.12 (m, 1H), 0.76 (d, 3H), 0.74 (d, 3H).

### Example 30

### Biotransformation of 3-formyl-5-methylhexanoic acid to pregabalin with recombinant ω-transaminases

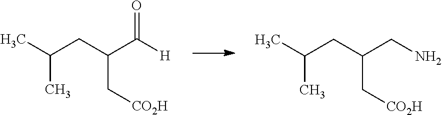

Transamination of 3-formyl-5-methylhexanoic acid to form Pregabalin was evaluated with various recombinant transaminases. Recombinant ω-transaminases from Vibrio fluvialis, Rhodobacter sphaeroides, and Paracoccus denitrificans were expressed in E. coli as follows: The pET28b ω-transaminase expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB+kanamycin Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C.

Reactions (0.5 mL) were carried out at 30° C. in potassium phosphate buffer (100 mM, pH 7.0), pyridoxal phosphate (2 mM), isopropylamine (150 mM), 3-formyl-5-methylhexanoic acid (50 mM) and ω-transaminase (40 mg wet cells/mL) from Vibrio fluvialis, Rhodobacter sphaeroides, or Paracoccus denitrificans. After 24 h, reaction samples (0.1 mL) were diluted with 0.4 mL acetonitrile:water (1:1, v/v). Aliquots (0.05 mL) of the diluted reaction samples were treated with saturated aqueous sodium bicarbonate (0.01 mL) and Marfey's reagent (N-α-(2,4-dinitro-5-fluorophenyl)alaninamide, 0.2 mL of 5 g/L solution in acetonitrile) at 40° C. After 1 h, the derivatization reactions were quenched with 0.01 mL 1N aqueous hydrochloric acid and diluted with 0.23 mL of acetonitrile. The derivatized reaction samples were analyzed by UPLC (column: BEH C18, 50 mm×2.1 mm id, gradient elution: 70% A:30% B to 55% A:45% B in 5 min (A=1% triethylamine (pH 3 with phosphoric acid); B=acetonitrile), flow rate: 0.8 mL/min, column temperature: 30° C., detection: 210-400 nm) to give the results shown in Table 5.

TABLE 5

| | | Accession | % Yield | % ee (S)- |
|---|---|---|---|---|
| Entry | Enzyme | Number | Pregabalin | Pregabalin |
| 1 | Vibrio fluvialis | AEA39183 | 37.5 | 60.6 |
| | ω-transaminase | | | |
| 2 | Rhodobacter sphaeroides | YP_001043908 | 24 | 84.4 |
| | ω-transaminase | | | |
| 3 | Paracoccus denitrificans | YP_917746 | 44 | 60.6 |
| | ω-transaminase | | | |

### Example 31

### Biotransformation of 3-formyl-5-methylhexanoic acid to pregabalin with recombinant ω-transaminases

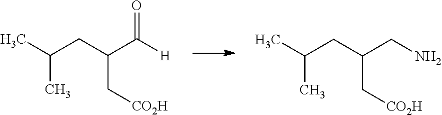

Recombinant variants of Vibrio fluvialis were tested for the reductive amination of 3-formyl-5-methylhexanoic acid to form Pregabalin. Variants of V. fluvialis ω-transaminase (Accession number AEA39183) were expressed in E. coli as follows: QuikChange Site-directed Mutagenesis kits from Stratagene (La Jolla, Calif., USA) was used to create V. fluvialis aminotransferase variants as directed. Primers were ordered from Integrated DNA Technologies (Coralville, Iowa, USA). The pET28b ω-transaminase expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB+kanamycin Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C.

Reactions (0.5 mL) were carried out at 30° C. in potassium phosphate buffer (100 mM, pH 7.0) with pyridoxal phosphate (2 mM), isopropylamine (300 mM), 3-formyl-5-methylhexanoic acid (100 mM) and V. fluvialis ω-transaminase wild-type or variants (40 mg wet cells/mL). After 28 h, reaction samples (0.1 mL) were diluted with 0.4 mL acetonitrile:water (1:1, v/v). Aliquots (0.05 mL) of the diluted reaction samples were treated with saturated aqueous sodium bicarbonate (0.01 mL) and Marfey's reagent (N-α-(2,4-dinitro-5-fluorophenyl)alaninamide, 0.2 mL of 5 g/L solution in acetonitrile) at 40° C. After 1 h, the derivatization reactions were quenched with 0.01 mL 1N aqueous hydrochloric acid and diluted 0.23 mL of acetonitrile. The derivatized reaction samples were analyzed by UPLC (column: BEH C18, 50 mm×2.1 mm id, gradient elution: 70% A:30% B to 55% A:45% B in 5 min (A=1% triethylamine (pH 3 with phosphoric acid); B=acetonitrile), flow rate: 0.8 mL/min, column temperature: 30° C., detection: 210-400 nm) to give the results shown in Table 6.

TABLE 6

| | V. fluvialis | | |
|---|---|---|---|
| | ω-transaminase | % | |
| Entry | variant | Conversion | % ee |
| 1 | W57F/K163L/R415F | 6.1 | 81.1 |
| 2 | Y165F | 9.5 | 75.3 |
| 3 | W147N | 10.0 | 75.0 |
| 4 | A228G | 32.3 | 74.9 |
| 5 | N166V | 16.6 | 74.5 |
| 6 | W57F/A228G | 33.3 | 73.9 |
| 7 | V156M | 13.2 | 70.3 |
| 8 | S159A | 7.9 | 70.2 |
| 9 | W57F/R415F | 10.7 | 68.5 |
| 10 | R415F | 11.8 | 68.1 |
| 11 | M59N | 10.1 | 68.0 |
| 12 | P301K | 19.1 | 67.6 |
| 13 | Y113F | 7.9 | 65.2 |
| 14 | F86G | 32.1 | 64.1 |
| 15 | I254V | 32.1 | 64.0 |
| 16 | H326N | 15.9 | 63.8 |
| 17 | C414I | 10.1 | 63.0 |
| 18 | W57F/K163L/V153A | 46.1 | 41.2 |
| 19 | W57F/K163L | 43.5 | 36.1 |
| 20 | D21Y | 45.1 | 34.2 |
| 21 | W57F/D21Y | 46.3 | 34.0 |
| 22 | M294V | 42.4 | 33.2 |
| 23 | W57F/M294V | 41.2 | 32.5 |
| 24 | W57F/V177I | 39.9 | 29.0 |
| 25¹ | Vat 565 | 20.0 | 90.5 |
| 26¹ | Vfat665 | 15.0 | 93.1 |
| 27¹ | Vfat701 | 9.0 | 94.4 |
| 28¹ | Vfat707 | 7.5 | 95.7 |
| 29¹ | Vfat747 | 50.0 | 97.5 |
| 30¹ | Vfat825 | 75.0 | 95.7 |
| 31¹ | Vfat 850 | 81.3 | 94.6 |
| 32¹ | Vfat 875 | 80.5 | 95.1 |
| 33¹ | Vfat888² | 95.0 | 95.5 |

Note
¹Entry 25 thru 33—reactions were performed using 400 mM of 3-formyl-5-methylhexanoic acid, 3 mM PLP, 800 mM IPM at 45° C.

2: Vfat 888:
DNA SEQUENCE

(SEQ ID NO. 10)

ATGAATAAACCACAAAGCTGGGAAGCGCGTGCTGAAACTTACTCTCTGTA
CGGCTTCACTGATATGCCATCTCTGCACCAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTAACAACGCATAC
CACGGTGTAACTGCTGTTTCCGCTTCCATGACGGGTCTGCCGTACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGGCGAAACGCCTGGAAACGGCGATCGAAGCTATCGAAGAGTT
CCCGCACGGCTTTACGGCCAGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
GAGCGTATCGCTAACACCTGTACCGACCTGGGCCTGATCTGTAGCCCGAT
GGGTCAGTCCGTTATCCTGTGCCCGCCGTTCATCCTGACCGAGGCGCAAA
TGGATGAGATGTTTGACAAACTGGAGAAGGCTCTGGACAAAGTCTTTGCG
GAGGTGGCGTAA

### Amino Acid Sequence

(SEQ ID NO. 2)

MNKPQSWEARAETYSLYGFTDMPSLHQRGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELAKRLETAIEAIEEFPHGFTASGHPVGCAIALKAIDVVMNEGLAENVR
RLAPRFEERLKHIAERPNIGEYRGIGFMWALEAVKDKASKTPFDGNLSVS
ERIANTCTDLGLICSPMGQSVILCPPFILTEAQMDEMFDKLEKALDKVFA
EVA

### Example 32

### Enzymatic reduction of (E)-3-formyl-5-methylhex-2-enoic acid to 3-formyl-5-methylhexanoic acid and in situ conversion to pregabalin with ω-transaminase

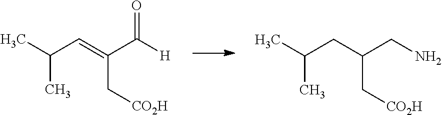

Reduction of (E)-3-formyl-5-methylhex-3-enoic acid to 3-formyl-5-methylhexanoic acid with pentaerythritol tetranitrate reductase and in situ conversion to pregabalin was evaluated with various ω-transaminases. Recombinant ω-transaminases from Vibrio fluvialis, Rhodobacter sphaeroides, and Paracoccus denitrificans were expressed in E. coli as follows: The pET28b ω-transaminase expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB+kanamycin Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C.

Reactions (0.5 mL) were carried out at 30° C. in potassium phosphate buffer (100 mM, pH 7.0) with pentaerythritol tetranitrate reductase (40 mg wet cells/mL), Lactobacillus brevis alcohol dehydrogenase (32 U/mL), NADP⁺ (0.02 mM), 2-propanol (3 vol %), pyridoxal phosphate (2 mM), isopropylamine (100 mM), (E)-3-formyl-5-methylhex-2-enoic acid (20 mM) and ω-transaminase (40 mg wet cells/mL) from Vibrio fluvialis, Rhodobacter sphaeroides, or Paracoccus denitrificans. After 43 h, reaction samples (0.1 mL) were diluted with 0.1 mL acetonitrile:water (1:1, v/v). Aliquots (0.1 mL) of the diluted reaction samples were treated with saturated aqueous sodium bicarbonate (0.01 mL) and Marfey's reagent (N-α-(2,4-dinitro-5-fluorophenyl)alaninamide, 0.4 mL of 5 g/L solution in acetonitrile) at 40° C. After 1 h, the derivatization reactions were quenched with 0.1 mL 1N aqueous hydrochloric acid. The derivatized reaction samples were analyzed by UPLC (column: BEH C18, 50 mm×2.1 mm id, gradient elution: 70% A:30% B to 55% A:45% B in 5 min (A=1% triethylamine (pH 3 with phosphoric acid); B=acetonitrile), flow rate: 0.8 mL/min, column temperature: 30° C., detection: 210-400 nm) to give the results shown in Table 7.

TABLE 7

| | | Accession | % Yield | % ee (S)- |
|---|---|---|---|---|
| Entry | Enzyme | Number | Pregabalin | Pregabalin |
| 1 | Vibrio fluvialis | AEA39183 | 60.8 | 42.0 |
| | ω-transaminase | | | |
| 2 | Rhodobacter sphaeroides | YP_001043908 | 60.4 | 54.6 |
| | ω-transaminase | | | |
| 3 | Paracoccus denitrificans | YP_917746 | 33.6 | 61.5 |
| | ω-transaminase | | | |

### Example 33

### Enzymatic reduction of (E)-3-formyl-5-methylhex-2-enoic acid to 3-formyl-5-methylhexanoic acid and in-situ conversion to pregabalin with V. fluvialis ω-transaminase

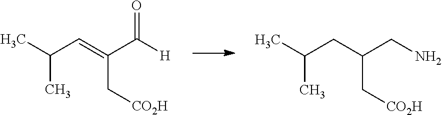

Reduction of (E)-3-formyl-5-methylhex-3-enoic acid to 3-formyl-5-methylhexanoic acid with pentaerythritol tetranitrate reductase and in-situ conversion to pregabalin was evaluated with variants of V. fluvialis aminotransferase. Variants of V. fluvialis co-transaminase (Accession number AEA39183) were expressed in E. coli as follows: QuikChange Site-directed Mutagenesis kits from Stratagene (La Jolla, Calif., USA) was used to create V. fluvialis aminotransferase variants as directed. Primers were ordered from Integrated DNA Technologies (Coralville, Iowa, USA). The pET28b ω-transaminase expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB+kanamycin Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C. Reactions (0.5 mL) were carried out at 30° C. in potassium phosphate buffer (100 mM, pH 7.0) with pentaerythritol tetranitrate reductase (40 mg wet cells/mL), Lactobacillus brevis alcohol dehydrogenase (32 U/mL), NADP⁺ (0.1 mM), 2-propanol (3 vol %), pyridoxal phosphate (2 mM), isopropylamine (300 mM), (E)-3-formyl-5-methylhex-2-enoic acid (100 mM) and V. fluvialis ω-transaminase wild-type or variants (40 mg wet cells/mL). After 48 h, reaction samples (0.02 mL) were diluted with 0.18 mL acetonitrile:water (1:1, v/v). Aliquots (0.1 mL) of the diluted reaction samples were treated with saturated aqueous sodium bicarbonate (0.01 mL) and Marfey's reagent (N-α-(2,4-dinitro-5-fluorophenyl)alaninamide, 0.4 mL of 5 g/L solution in acetonitrile) at 40° C. After 1 h, the derivatization reactions were quenched with 0.1 mL 1N aqueous hydrochloric acid. The derivatized reaction samples were analyzed by UPLC (column: BEH C18, 50 mm×2.1 mm id, gradient elution: 70% A:30% B to 55% A:45% B in 5 min (A=1% triethylamine (pH 3 with phosphoric acid); B=acetonitrile), flow rate: 0.8 mL/min, column temperature: 30° C., detection: 210-400 nm) to give the results shown in Table 8.

TABLE 8

| | V. fluvialis transaminase | % Yield | % ee |
|---|---|---|---|
| Entry | Variant | Pregabalin | (S)-Pregabalin |
| 1 | M294V | 64.8 | 73.7 |
| 2 | T268M | 47.4 | 77.8 |
| 3 | P233L | 77.3 | 71 |
| 4 | N166V | 38.1 | 73.1 |
| 5 | C424A | 76.5 | 70.6 |
| 6 | L100M | 83.7 | 71.4 |
| 7 | S283A | 68.1 | 71 |
| 8 | L417M | 68.7 | 71.8 |
| 9 | Wild-type | 62.7 | 61.5 |

### Example 34

### Alternative Variants of Vibrio fluvalis ω-Transaminase

The following further recombinant variants of Vibrio fluvialis ω-transaminase were expressed in E. coli as follows: The pET28b ω-transaminase expression constructs were transformed into BL21(DE3) E. coli (Stratagene, Agilent Technologies, Santa Clara, Calif., USA) as directed and overnight cultures were grown in LB+kanamycin media. The LB cultures were used to inoculate expression cultures grown in Overnight Express Instant TB+kanamycin Medium (Novagen, EMD Chemicals, Gibbstown, N.J., USA). Cultures were incubated for 20 h at 30° C., and the cells were harvested by centrifugation (4000×g, 30 min, 4° C.) and stored at −20° C.

### Example 34a

### Vfat906

DNA Sequence:

(SEQ ID NO. 11)

ATGAATAAACCACAAAGCTGGGAAGCGCGTGCTGAAACTTACTCTCTGTA
CGGCTTCACTGATATGCCATCTCTGCACgAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTaacAACGCATAC
CACGGTGTAACTGCTGTTTCCGCTTCCATGACGGGTCTGCCGTACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCggcGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
aaaCGTATCGCTAACACCTGTcagGACCTGGGCCTGATCTGTAGCgCGCT
GGGTCAGTCCGTTATCCTGTGCCCGCCGTTCATCCTGACCGAGGCGCAAA
TGGATGAGATGTTTGACAAACTGGAGAAGGCTCTGGACAAAGTCTTTGCG
GAGGTGGCGTAA
Amino acid sequence:

(SEQ ID NO. 3)

MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSALGQSVILCPPFILTEAQMDEMFDKLEKALDKVFA
EVA

### Example 34b

### Vfat999

DNA Sequence:

(SEQ ID NO. 12)

ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACGTGGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGCT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA
Amino acid sequence:

(SEQ ID NO. 4)

MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSALGQSVILSPPFILTEAQMDEMFDKLEKALDKVFA
EVA

### Example 34c

### Vfat1010

DNA Sequence:

(SEQ ID NO. 13)

ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTATGCCGCACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGCACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA
Amino acid sequence:

(SEQ ID NO. 5)

MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGMPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PALSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSAMGQSVILSPPFILTEAQMDEMFDKLEKALDKVFA
EVA

### Example 34d

### Vfat1020

DNA Sequence:

(SEQ ID NO. 14)

ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACGTGGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGGGTTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA
Amino acid sequence:

(SEQ ID NO. 6)

MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLGCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICAAMGQSVILSPPFILTEAQMDEMFDKLEKALDKVFA
EVA

### Example 34e

### Vfat1030

DNA Sequence:

(SEQ ID NO. 15)

ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACATCGTGGACGTCCACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGAGCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA
Amino acid sequence:

(SEQ ID NO. 7)

MNKPQSWEARAETYSLYGFTDMPSLHERGTVVVTHGEGPYIVDVHGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLSCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSAMGQSVILSPPFILTEAQMDEMFDKLEKALDKVFA
EVA

### Example 35

### Biotransformation of 3-isobutylidene-2-oxopentanedioic acid to Pregabalin

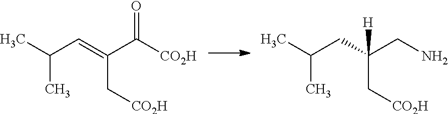

The genes for the Pseudomonas putida benzoylformate decarboxylase, Enterobacter cloacae pentaerythritol tetranitrate reductase, Lactobacillus brevis alcohol dehydrogenase, and Vibrio fluvialis ω-transaminase were cloned into expression vector pDSTRC52 (Pfizer Inc., USA) and put into the expression strain E. coli BDG62 (Pfizer Inc., USA). The culture was grown and enzyme production was induced as described in Example 22. Enzyme expression was determined by polyacrylamide gel electrophoresis using Novex gels and stains (Invitrogen Corporation Carlsbad, Calif.).

Reactions (1.0 mL) were carried out at 40° C. in phosphate buffer (100 mM, pH 6.4) with P. putida decarboxylase, V. fluvialis transaminase, L. brevis alcohol dehydrogenase, E. cloacae pentaerythritol tetranitrate reductase, and NADP (0.1 mM) in one cell. 200 μL 1M 3-isobutylidene-2-oxopentanedioic acid, 100 μL 1 mM ThDp+25 mM MgSO4, 40 μL 50 mM PLP, 30 μL isopropanol, 250 μL 2M isopropylamine. Adjust pH to 6.4 for 24 hours, then adjust pH to 6.8 for additional 24 hours. An aliquot (0.5 mL) of the reaction was treated with 1M aqueous sodium bicarbonate (0.05 mL) and Marfey's reagent (N-α-(2,4-dinitro-5-fluorophenyl)alaninamide, 0.5 mL of 5 g/L solution in acetonitrile) at 40° C. After 1 h, the derivatization reactions were quenched with 0.05 mL 1N aqueous hydrochloric acid. The derivatized reaction samples were filtered and analyzed by UPLC (column: Agilent Eclipse Plus C18 column (100 mm×3.0 mm, 1.8 μm) eluted with 0.1% trifluoroacetic acid in water:acetonitrile (60:40, v/v) at 1.3 mL/min. The column was maintained at 30° C. and the effluent was monitored at 340 nm and ES⁺ mass spectroscopy. The reaction yielded a small amount of Pregabalin of which 82% was the desired S-isomer.

### Example 36

### Biotransformation of 3-(2-methylpropyl)-2-oxopentanedioic acid to Pregabalin

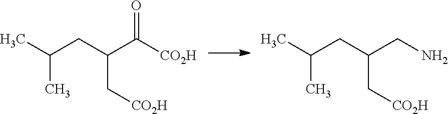

The genes for the Pseudomonas putida benzoylformate decarboxylase and Vibrio fluvialis ω-transaminase were cloned into expression vector pDSTRC52 (Pfizer Inc., USA) and put into the expression strain E. coli BDG62 (Pfizer Inc., USA). The culture was grown and enzyme production was induced as described in Example 22. Enzyme expression was determined by polyacrylamide gel electrophoresis using Novex gels and stains (Invitrogen Corporation Carlsbad, Calif.).

Reactions (2.0 mL) were carried out at 40° C. in phosphate buffer (100 mM, pH 6.4) with 500 μL of culture (42.5 mg dry cell weight) from a 24 hour fermentation tanks with P. putida decarboxylase and V. fluvialis ω-transaminase cloned in one plasmid, 400 μL 0.5M 3-(2-methylpropyl)-2-oxopentanedioic acid, 200 uL 10×ThDP (final 0.1 mM) and MgSO₄ (final 2.5 mM), 80 μL 50 mM PLP (final 2 mM), 300 μL 2M isopropylamine (final 0.3M). Adjust pH to 6.4 and incubate at 45° C. for 24 hours, then adjust pH to 6.8 for additional 24 hours. An aliquot (0.5 mL) of the reaction was treated with 1M aqueous sodium bicarbonate (0.05 mL) and Marfey's reagent (N-α-(2,4-dinitro-5-fluorophenyl)alaninamide, 0.5 mL of 5 g/L solution in acetonitrile) at 40° C. After 1 h, the derivatization reactions were quenched with 0.05 mL 1N aqueous hydrochloric acid. The derivatized reaction samples were filtered and analyzed by UPLC (column: Agilent Eclipse Plus C18 column (100 mm×3.0 mm, 1.8 μm) eluted with 0.1% trifluoroacetic acid in water:acetonitrile (60:40, v/v) at 1.3 mL/min. The column was maintained at 30° C. and the effluent was monitored at 340 nm and ES⁺ mass spectroscopy. The reaction yielded 100 μg/mL of Pregabalin of which 65% was the desired S-isomer.

### Example 37

### Preparation of (S)-Pregabalin via hydrolysis of 5-methoxy-4-(2-methylpropyl)-dihydrofuran-2(3H)-one and enzymatic transamination

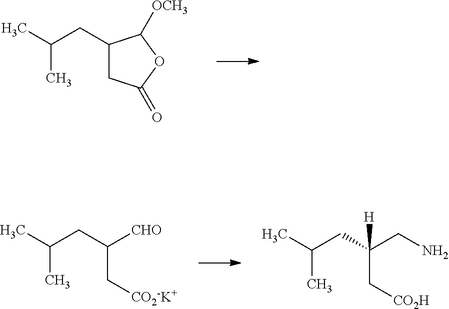

5-Methoxy-4-(2-methylpropyl)-dihydrofuran-2(3H)-one (2.58 g, 15 mmol, see example 13) was suspended in DIW (5.2 g) and cooled in an ice/water bath. Aq KOH (50% w/w, 1.77 g, 1.05 eq) was added dropwise via syringe over 5 mins. The reaction was removed from the ice/water bath and stirred at rt for 90 mins. The pH was adjusted to 7.0 using formic acid. The reaction mixture was then used as feedstock in the subsequent transaminase reaction.

Transaminase enzyme solution (12.5 g), PLP (35 mg), DIW (15 mL) and 4.0M isopropylamine.HCl aq soln (7.5 mL, 30 mmol) were charged to a 100 mL flask and warmed to 45° C. The hydrolysis reaction was added in one portion followed by DIW (6 mL, used as a vessel rinse). The pH of the reaction was adjusted to 7.25 using neat isopropylamine and the reaction was stirred at 45° C. until reaction completion. The reaction mixture was then heated to an internal temperature of 55° C. and the pH adjusted to 4.0 using 95% formic acid. Darco carbon (125 mg) was added and the mixture was allowed to cool to room temperature before cooling on ice/water for 20 minutes. The mixture was then filtered through Whatman paper no. 3. The filtrate was concentrated to ⅓ of its weight and then heated to 55° C. The pH of the solution was then adjusted to pH 7.5 using 50% KOH after which the solution was cooled to ambient and then to 0-5° C. in an ice/water bath. Precipitation of product was observed in the cooldown. The slurry was filtered and washed with DIW/EtOH (10 mL, 1:1, 0° C.). The white precipitate was dried for 12 hours in a vacuum oven (45° C.) to yield (S)-Pregabalin in 61% yield, 98.6% w/w purity and 99.8% ee (preferred S-isomer).

### Example 38

### Preparation of (S)-Pregabalin via hydrolysis of 5-butoxy-4-(2-methylpropyl)-dihydrofuran-2(3H)-one and enzymatic transamination

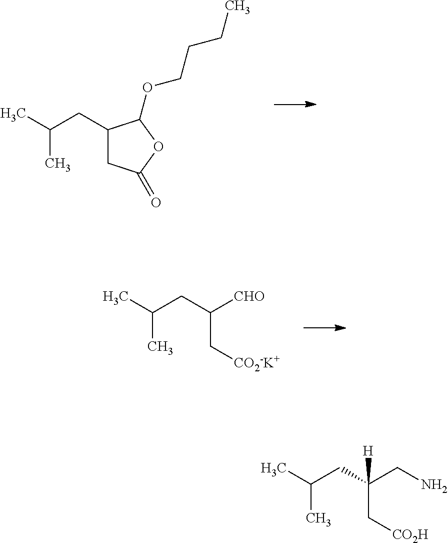

5-Butoxy-4-(2-methylpropyl)-dihydrofuran-2(3H)-one (3.21 g, 15 mmol, see example 13C) was suspended in DIW (8.5 g) and cooled in an ice/water bath. Aq KOH (50% w/w, 2.02 g, 1.2 eq) was added dropwise via syringe over 5 mins. The reaction was removed from the ice/water bath and stirred at rt for 90 mins. The reaction pH was then adjusted to pH 7.0 using formic acid, and the reaction mixture was then used as feedstock in the subsequent transaminase reaction.

Transaminase enzyme solution (12.5 g), PLP (35 mg), DIW (15 mL) and 4.0M isopropylamine.HCl solution (7.5 mL, 30 mmol) were charged to a 100 mL flask and warmed to 45° C. The hydrolysis reaction (see above) was added in one portion followed by DIW (6 mL, used as a vessel rinse). The pH of the reaction was adjusted to 7.25 using neat isopropylamine and the reaction was stirred at 45° C.

The reaction mixture was heated to an internal temperature of 55° C. and pH adjusted to 4.0 using 95% formic acid. Darco carbon (125 mg) was added and the mixture was allowed to cool to room temperature before being cooled on ice/water for 20 minutes. The mixture was then filtered through Whatman paper no 3. The filtrate was concentrated to ⅓ of its weight then heated to 55° C. The pH of the solution was then adjusted to pH 7.5 using 50% KOH after which the solution was cooled to ambient and then to 0-5° C. in an ice/water bath. Precipitation of product was observed in the cooldown. The slurry was filtered and washed with DIW/EtOH (10 mL, 1:1, 0° C.). The white precipitate was dried for 12 hours in a vacuum oven (45° C.) to yield (S)-Pregabalin in 51% yield, 98.4% w/w purity and 99.9% e.e preferred S-isomer.

### Example 39

### Preparation of (S)-Pregabalin from 5-hydroxy-4-(2-methylpropyl)-dihydrofuran-2(3H)-one via enzymatic transamination

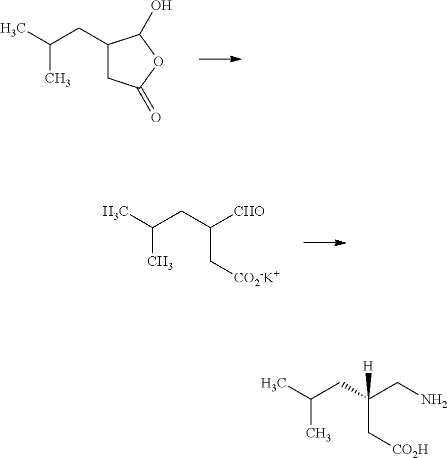

5-Hydroxy-4-(2-methylpropyl)-dihydrofuran-2(3H)-one (2.0 g, 12.5 mmol) was suspended in DIW (8.5 g) and cooled in an ice/water bath. Potassium carbonate (0.863 g, 6.3 mmol) was added portion wise over the course of 5 mins. The reaction was removed from the ice/water bath and stirred at rt for 90 mins. The pH was adjusted to 7.0 using formic acid and the reaction mixture was then used as feedstock in the subsequent transaminase reaction.

Transaminase enzyme solution (10.4 g), PLP (30 mg), DIW (12.5 mL) and aq 4.0M isopropylamine.HCl aq soln (6.3 mL, 30 mmol) were charged to a 100 mL flask and warmed to 45° C. The hydrolysis reaction was added in one portion followed by DIW (5 mL, used as a vessel rinse). The pH of the reaction was adjusted to 7.25 using neat isopropylamine and the reaction was stirred at 45° C. The reaction mixture was heated to an internal temperature of 55° C. and adjusted to pH 4.0 using 95% formic acid. Darco carbon (125 mg) was added and the mixture was allowed to cool to room temperature before being cooled on ice/water for 20 minutes. The mixture was then filtered through Whatman paper no 3. The filtrate was concentrated to ⅓ of its weight then heated to 55° C. The pH of the solution was then adjusted to pH 7.5 using 50% KOH after which the solution was cooled to ambient and then to 0-5° C. in an ice/water bath. Precipitation of product was observed in the cooldown. The slurry was filtered and washed with DIW/EtOH (10 mL, 1:1, 0° C.). The white precipitate was dried for 12 hours in a vacuum oven (45° C.) to yield (S)-Pregabalin in 61% yield, 98.3% w/w purity and 99.9% e.e of the preferred S-isomer.

### Example 40

### Preparation of (R/S)-3-aminomethyl-5-methylhexanoic acid via Reductive amination of 5-hydroxy-4-(2-methylprop-1-en-1-yl)furan-2(5H)-one

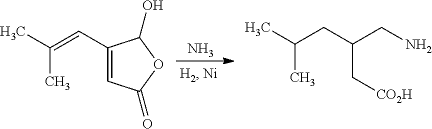

A 0.01M solution of 5-hydroxy-4-(2-methylprop-1-en-1-yl)furan-2(5H)-one in 30% aqueous ammonia solution was hydrogenated (10 bar, ambient temperature) for 48 h in the presence of 10 mol % Raney Nickel catalyst. The catalyst was filtered and the solution concentrated to leave a solid. The product was isolated by addition of hydrochloric acid to a methanol suspension and found to be pure 3-aminomethyl-5-methylhexanoic acid hydrochloride.

The use of palladium as a catalyst gave mixtures of 3-aminomethyl-5-methylhexanoic acid and the corresponding secondary amine, 3-[(2-carboxymethyl-4-methyl-pentylamino)-methyl]-5-methyl-hexanoic acid.

### Example 41

### Conversion of R-3-aminomethyl-5-methylhexanoic acid to 5-hydroxy-4-(2-methylpropyl)-3,4-dihydro-5H-2-furanone using a transaminase

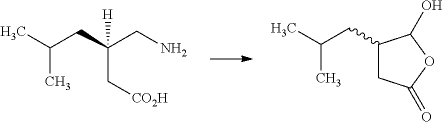

A solution of R-3-aminomethyl-5-methylhexanoic acid in D.I. water at pH 7.5/45° C. is stirred with a transaminase enzyme lysate, or whole cell preparation containing pyridoxal phosphate (PLP) and acetone. The isopropylamine produced is removed via a nitrogen sweep. Analysis of the solution after 24 h show 3-aminomethyl-5-methyl-hexanoic acid with a lower e.e. and the presence of compound 5-hydroxy-4-(2-methylpropyl)-3,4-dihydro-5H-2-furanone. The lowering of the chiral purity of the 5-hydroxy-4-(2-methylpropyl)-3,4-dihydro-5H-2-furanone is due to the selective conversion of (4S)-5-hydroxy-4-(2-methylpropyl)-3,4-dihydro-5H-2-furanone to S-pregabalin.

### Example 42

### De-Racemisation of Rac-Pregabalin Using a Transaminase

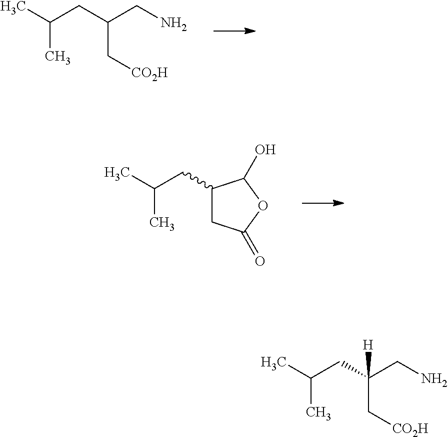

A solution of racemic pregabalin in DIW at pH 7.5 is treated with a suitable transaminase lysate. PLP is added and the reaction progressed for 12 h at 45° C. with a nitrogen sweep. Then, isopropylamine is added and the reaction is continued for a further 12 h. The product is isolated as in Example 38 to afford enantiomerically enriched pregabalin.

It is also possible to effect the transformations exemplified in Examples 41 and 42 using a suitable amine oxidase/imine reductase enzyme system with a co-factor such as NADP.

### SEQUENCE LISTING

SEQ ID NO. 1

Generic Vfat aa sequence
MNKPQSWEARAETYSLYGFTDMPSLHX²⁷RGTVVVTHGEGPYX⁴¹VDV
X⁴⁵GRRYLDANSGLYNMVAGFDHKGLIDAAKAQYERFPGYHSFFGRMSDQ
TVMLSEKLVEVSPFDSGRVFYTNSGSEANDTMVKMLWFLHAAEGKPQKRK
ILTRX¹⁴⁷NAYHGVTAVSASMTGX¹⁶³PX¹⁶⁵NSVFGLPLPGFVHL
X¹⁸⁰CPHYWRYGEEGETEEQFVARLARELEETIQREGADTIAGFFAEP
VMGAGGVIPPAKGYFQAILPILRKYDIPVISDEVICGFGRTGNTWGCVTY
DFTPDAIISSKNLTAGFFPVGAVILGPELX³⁰⁴KRLETAIEAIEEFPHGF
TAX³²⁴GHPVGCAIALKAIDWMNEGLAENVRRLAPRFEERLKHIAERPN
IGEYRGIGFMWALEAVKDKASKTPFDGNLSVSX⁴⁰¹RIANTCX⁴⁰⁸D
LGLICX⁴¹⁵X⁴¹⁶X⁴¹⁷GQSVILX⁴²⁴PPFILTEAQMDEMFDKLEKALD
KVFAEVA

X²⁷ is selected from glutamine (Q) and glutamic acid (E); X⁴¹ is selected from isoleucine (I) and valine (V); X⁴⁵ is selected from asparigine (N) and histidine (H); X¹⁴⁷ is selected from asparigine (N) and glutamine (Q); X¹⁶³ is selected from leucine (L) and methionine (M); X¹⁶⁵ is selected from tyrosine (Y) and histidine (H); X¹⁸⁰ is selected from threonine (T); glycine (G) and serine (S); X³⁰⁴ is selected from alanine (A) and serine (S); X³²⁴ is selected from glycine (G) and serine (S); X⁴⁰¹ is selected from lysine (K) and glutamic acid (E); X⁴⁰⁸ is selected from threonine (T) and glutamine (Q); X⁴¹⁵ is selected from serine (S) and alanine (A); X⁴¹⁶ is selected from proline (P) and alanine (A); X⁴¹⁷ is selected from leucine (L) and methionine (M); and X⁴²⁴ is selected from cysteine (C) and serine (S).

SEQ ID NO. 2

Vfat888 aa sequence
MNKPQSWEARAETYSLYGFTDMPSLHQRGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELAKRLETAIEAIEEFPHGFTASGHPVGCAIALKAIDWMNEGLAENVRR
LAPRFEERLKHIAERPNIGEYRGIGFMWALEAVKDKASKTPFDGNLSVSE
RIANTCTDLGLICSPMGQSVILCPPFILTEAQMDEMFDKLEKALDKVFAE
VA

SEQ ID NO. 3

Vfat906 aa sequence
MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICSALGQSVILCPPFILTEAQMDEMFDKLEKALDKVFAE
VA

SEQ ID NO. 4

Vfat999 aa sequence
MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSALGQSVILSPPFILTEAQMDEMFDKLEKALDKVFA
EVA

SEQ ID NO. 5

Vfat1010 aa sequence
MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGMPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PALSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSAMGQSVILSPPFILTEAQMDEMFDKLEKALDKVFA
EVA

SEQ ID NO. 6

Vfat1020 aa sequence
MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLGCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICAAMGQSVILSPPFILTEAQMDEMFDKLEKALDKVFA
EVA

SEQ ID NO. 7

Vfat1030 aa sequence
MNKPQSWEARAETYSLYGFTDMPSLHERGTVVVTHGEGPYIVDVHGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLSCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDVVMNEGLAENVR
RLAPRFEERLKHIAERPNIGEYRGIGFMWALEAVKDKASKTPFDGNLSVS
KRIANTCQDLGLICSAMGQSVILSPPFILTEAQMDEMFDKLEKALDKVFA
EVA

SEQ ID NO. 8

Lycopersicon esculentum (tomato)
12-Oxophytodienoate Reductase 1 protein
sequence:
MENKVVEEKQVDKIPLMSPCKMGKFELCHRWLAPLTRQRSYGYIPQPHAI
LHYSQRSTNGGLLIGEATVISETGIGYKDVPGIVVTKEQVEAWKPIVDAV
HAKGGIFFCQIWHVGRVSNKDFQPNGEDPISCTDRGLTPQIRSNGIDIAH
FTRPRRLTTDEIPQIVNEFRVAARNAIEAGFDGVEIHGAHGYLIDQFMKD
QVNDRSDKYGGSLENRCRFALEIVEAVANEIGSDRVGIRISPFAHYNEAG
DTNPTALGLYMVESLNKYDLAYCHVVEPRMKTAWEKIECTESLVPMRKAY
KGTFIVAGGYDREDGNRALIEDRADLVAYGRLFISNPDLPKRFELNAPLN
KYNRDTFYTSDPIVGYTDYPFLETMT

SEQ ID NO. 9

Lycopersicon esculentum (tomato)
12-Oxophytodienoate Reductase 1 codon optimized
sequence:
ATGGAAAACAAAGTTGTGGAAGAAAAACAGGTTGATAAAATCCCGCTGAT
GAGCCCGTGTAAAATGGGTAAATTCGAGCTGTGTCATCGCGTTGTACTGG
CACCGCTGACTCGTCAGCGTTCTTATGGTTACATTCCGCAGCCGCACGCA
ATCCTGCATTACTCTCAGCGCAGCACCAACGGTGGCCTGCTGATCGGTGA
AGCAACCGTGATCAGCGAAACTGGCATCGGTTACAAAGATGTGCCGGGTA
TCTGGACGAAAGAGCAGGTTGAGGCCTGGAAACCGATCGTCGACGCGGTG
CATGCCAAAGGTGGTATTTTCTTTTGTCAGATCTGGCACGTTGGTCGTGT
ATCCAACAAAGATTTTCAGCCGAACGGCGAAGATCCGATTTCCTGTACTG
ACCGCGGCCTGACCCCGCAGATCCGTTCCAACGGCATTGACATTGCCCAC
TTCACCCGTCCACGTCGCCTGACTACTGACGAGATTCCGCAGATCGTGAA
CGAGTTCCGCGTTGCAGCGCGTAATGCTATTGAAGCGGGTTTCGATGGCG
TCGAGATTCATGGTGCCCACGGTTACCTGATCGACCAATTCATGAAAGAC
CAAGTTAACGACCGCAGCGATAAGTATGGCGGTTCTCTGGAGAACCGTTG
TCGCTTCGCGCTGGAAATCGTTGAAGCAGTAGCCAACGAGATTGGCTCCG
ACCGTGTTGGTATCCGTATCTCTCCATTCGCACACTACAACGAAGCGGGC
GACACTAACCCGACCGCACTGGGCCTGTATATGGTGGAGAGCCTGAATAA
ATACGACCTGGCGTATTGTCACGTGGTCGAGCCGCGCATGAAAACCGCCT
GGGAAAAGATTGAGTGCACCGAAAGCCTGGTGCCGATGCGTAAAGCCTAC
AAAGGCACCTTCATCGTAGCTGGTGGCTACGACCGTGAAGACGGTAACCG
CGCTCTGATCGAAGACCGTGCCGACCTGGTTGCGTACGGTCGTCTGTTCA
TCAGCAACCCAGACCTGCCGAAGCGTTTTGAACTGAACGCTCCGCTGAAC
AAATACAACCGTGACACTTTCTACACTTCCGACCCGATCGTTGGTTACAC
CGATTACCCGTTTCTGGAAACTATGACTTAATAA

SEQ ID NO. 10

Vfat 888 DNA SEQUENCE
ATGAATAAACCACAAAGCTGGGAAGCGCGTGCTGAAACTTACTCTCTGTA
CGGCTTCACTGATATGCCATCTCTGCACCAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTAACAACGCATAC
CACGGTGTAACTGCTGTTTCCGCTTCCATGACGGGTCTGCCGTACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGGCGAAACGCCTGGAAACGGCGATCGAAGCTATCGAAGAGTT
CCCGCACGGCTTTACGGCCAGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
GAGCGTATCGCTAACACCTGTACCGACCTGGGCCTGATCTGTAGCCCGAT
GGGTCAGTCCGTTATCCTGTGCCCGCCGTTCATCCTGACCGAGGCGCAAA
TGGATGAGATGTTTGACAAACTGGAGAAGGCTCTGGACAAAGTCTTTGCG
GAGGTGGCGTAA

SEQ ID NO. 11

Vfat 906 DNA SEQUENCE
ATGAATAAACCACAAAGCTGGGAAGCGCGTGCTGAAACTTACTCTCTGTA
CGGCTTCACTGATATGCCATCTCTGCACgAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTaacAACGCATAC
CACGGTGTAACTGCTGTTTCCGCTTCCATGACGGGTCTGCCGTACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCggcGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
aaaCGTATCGCTAACACCTGTcagGACCTGGGCCTGATCTGTAGCgCGCT
GGGTCAGTCCGTTATCCTGTGCCCGCCGTTCATCCTGACCGAGGCGCAAA
TGGATGAGATGTTTGACAAACTGGAGAAGGCTCTGGACAAAGTCTTTGCG
GAGGTGGCGTAA

SEQ ID NO. 12

Vfat 999 DNA SEQUENCE
ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACGTGGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGCT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA

SEQ ID NO. 13

Vfat 1010 DNA SEQUENCE
ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACATCGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTATGCCGCACAACTC
TGTATTCGGCCTGCCGCTGCCGGGTTTCGTTCACCTGACCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGCACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA

SEQ ID NO. 14

Vfat 1020 DNA SEQUENCE
ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACGTGGTGGACGTCAACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGGGTTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA

SEQ ID NO. 15

Vfat 1030 DNA SEQUENCE
ATGAATAAACCACAAAGCTGGGAAGCGCGTGCTGAAACTTACTCTCTGTA
CGGCTTCACTGATATGCCATCTCTGCACGAGCGTGGTACCGTGGTTGTCA
CCCACGGCGAGGGCCCATACATCGTGGACGTCCACGGTCGCCGTTACCTG
GACGCAAACTCCGGCCTGTACAATATGGTTGCCGGCTTCGACCACAAGGG
TCTGATCGACGCAGCAAAGGCCCAGTACGAACGCTTCCCGGGTTACCATA
GCTTCTTCGGTCGTATGTCTGATCAAACTGTTATGCTGAGCGAGAAACTG
GTAGAGGTGTCTCCATTCGACAGCGGTCGCGTGTTCTATACTAACTCCGG
CTCCGAGGCTAACGATACTATGGTGAAAATGCTGTGGTTTCTGCACGCCG
CAGAGGGCAAGCCGCAAAAACGCAAAATCCTGACTCGTCAAAACGCATAC
CACGGTGTAACTGCTGTTTCCGCTTCCATGACGGGTCTGCCGCACAACTC
TGTATTCGGCCTGCCGCTGCCGGGTTTCGTTCACCTGAGCTGTCCGCACT
ATTGGCGTTACGGCGAAGAAGGTGAAACCGAAGAGCAGTTTGTTGCTCGT
CTGGCCCGCGAGCTGGAGGAAACTATCCAACGTGAAGGCGCGGACACGAT
TGCGGGCTTCTTTGCTGAGCCGGTCATGGGCGCGGGCGGCGTAATCCCGC
CGGCGAAAGGTTACTTCCAGGCGATCCTGCCGATTCTGCGTAAGTACGAC
ATCCCGGTTATCTCTGATGAAGTTATCTGCGGCTTTGGTCGTACCGGTAA
TACTTGGGGTTGCGTTACCTATGACTTCACCCCGGATGCGATCATCTCCA
GCAAAAATCTGACCGCCGGTTTCTTTCCGGTTGGTGCTGTGATTCTGGGT
CCGGAACTGAGCAAACGCCTGGAAACGGCGATCGAAGCTATCGAAGAGTT
CCCGCACGGCTTTACGGCCGGCGGTCACCCGGTGGGTTGCGCTATCGCTC
TGAAAGCAATCGATGTTGTGATGAATGAGGGTCTGGCAGAGAACGTGCGC
CGCCTGGCACCGCGTTTTGAGGAGCGTCTGAAACACATTGCCGAACGTCC
GAACATCGGTGAATATCGTGGCATCGGTTTTATGTGGGCACTGGAGGCTG
TGAAAGACAAAGCATCTAAAACCCCATTCGATGGTAATCTGTCTGTGAGC
AAACGTATCGCTAACACCTGTCAGGACCTGGGCCTGATCTGTAGCGCGAT
GGGTCAGTCCGTTATCCTGAGCCCGCCGTTCATCCTGACCGAGGCGCAAA
TGGATGAGATGTTTGACAAACTGGAGAAGGCTCTGGACAAAGTCTTTGCG
GAGGTGGCGTAA

1. A compound according to formula (VI) 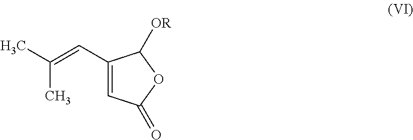
wherein R is selected from:
hydrogen,
(C₁-C₆)alkyl,
(C₁-C₆)haloalkyl,
(C₁-C₃)alkoxy(C₂-C₆)alkyl,
(C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy,
aryl(C₁-C₆)alkyl, wherein the aryl group may optionally be substituted with 1, 2 or 3 groups independently selected from halo, (C₁-C₃)alkyl, and (C₁-C₃)alkyloxy,
R¹—C(O)—, and
R²—SO₂—;
R¹ is selected from:
hydrogen,
(C₁-C₆)alkyl,
(C₁-C₆)haloalkyl,
(C₁-C₃)alkoxy(C₂-C₆)alkyl, (C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy, and
aryl(C₁-C₆)alkyl, wherein the aryl group may optionally be substituted with 1, 2 or 3 groups independently selected from halo, (C₁-C₃)alkyl, and (C₁-C₃)alkyloxy; and
R² is selected from:
(C₁-C₆)alkyl,
(C₁-C₆)haloalkyl,
(C₁-C₃)alkoxy(C₂-C₆)alkyl,
(C₂-C₆)alkenyl,
(C₃-C₁₀)cycloalkyl, which may optionally be substituted with 1, 2 or 3 groups independently selected from halo, (C₁-C₃)alkyl, and (C₁-C₃)alkyloxy,
(C₃-C₁₀)cycloalkyl(C₁-C₆)alkyl, wherein the cycloalkyl group may optionally be substituted with 1, 2 or 3 groups independently selected from halo, (C₁-C₃)alkyl, and (C₁-C₃)alkyloxy,
aryl, which may optionally be substituted with 1, 2 or 3 groups independently selected from halo, (C₁-C₃)alkyl, and (C₁-C₃)alkyloxy, and
aryl(C₁-C₆)alkyl, wherein the aryl group may optionally be substituted with 1, 2 or 3 groups independently selected from halo, (C₁-C₃)alkyl, and (C₁-C₃)alkyloxy.

2. The compound of claim 1 which is 5-hydroxy-4-(2-methyl-1-propenyl)-5H-2-furanone according to formula (VI^{A})

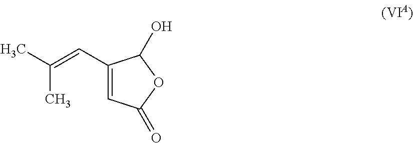

3. The compound according to claim 1 of formula (VI^{B}) 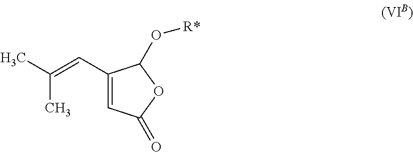
wherein R* is a chiral (C₅-C₁₅)hydrocarbon group.

4. The compound according to claim 3 wherein R* is selected from (R)- or (S)-α-methyl benzyl, (R)- or (S)-1-(1-naphthyl)ethyl, (R)- or (S)-1-(2-naphthyl)ethyl, menthyl and bornyl.

5. The compound according to claim 1 wherein R is R¹—C(O)— or R²—SO₂- and R¹ and R² are chiral (C₅-C₁₅) hydrocarbon groups.

6. A compound of formula (IX) 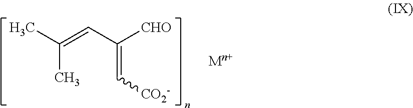
wherein:
n is 1 and M⁺ is selected from Li⁺, Na⁺, K⁺, Rb⁺, NH₄⁺, ((C₁-C₃)alkyl)NH₃⁺, ((C₁-C₃)alkyl)₂N H₂⁺, ((C₁-C₃)alkyl)₃NH⁺ and ((Cᵣ C₃)alkyl)₄N⁺; or
n is 2 and M²⁺ is selected from Mg²⁺, Ca²⁺ and Zn²⁺.

7. The compound according to claim 6 wherein n is 1 and M⁺ is selected from NH₄⁺ and ((C₁-C₃)alkyl)NH₃⁺.

8. The compound according to claim 6 wherein n is 1 and M⁺ is selected from Li⁺, Na⁺ and K⁺.

9. A compound of formula (VII) 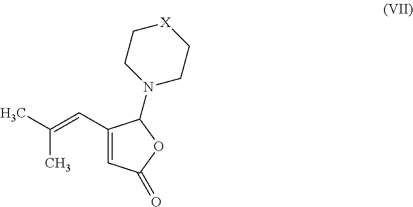
wherein —X— represents a single bond, —CH₂—, —O—; —NH—, —N((C₁-C₃)alkyl)-, —N(benzyl)-, or

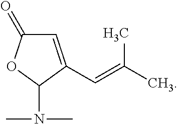

10. The compound according to claim 9 selected from:
4-(2-methylpropenyl)-5-pyrrolidin-1-yl-5H-furan-2-one;
4-(2-methylpropenyl)-5-piperidin-1-yl-5H-furan-2-one;
4-(2-methylpropenyl)-5-morpholin-4-yl-5H-furan-2-one; or
1,4-bis-(4-(2-methylpropenyl)-5H-furan-2-on-5-yl)piperazine.

11. A process for the manufacture of the compound of formula (VI^{A}) according to claim 2 comprising the step of treating a compound of formula (VII) 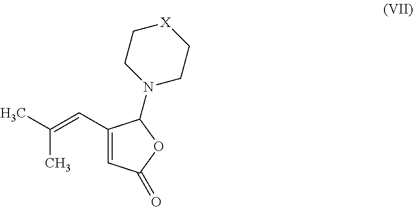
wherein —X— represents a single bond, —CH₂—, —O—, —NH—, —N((C₁-C₃)alkyl)-, —N(benzyl)-, or 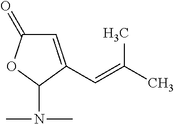
with water in the presence of an acid catalyst.

12. A process for the manufacture of a compound of formula (VI) according to claim 1 wherein R is other than hydrogen, R¹—C(O)—, and R²—SO₂—, comprising the step of treating a compound of formula (VI^{A}) with an alcohol R—OH in the presence of an acid catalyst.

13. A process for the manufacture of a compound of formula (VI) according to claim 1 wherein R is other than hydrogen, R¹—C(O)—, and R²—SO₂—, comprising the step of treating a compound of formula (VII) with an alcohol R—OH in the presence of stoichiometric acid.

14. A process for the manufacture of a compound of formula (VI) according to claim 1 wherein R is R¹—C(O)—, comprising the steps of treating a compound of formula (VI^{A}) with an acid chloride R¹—C(O)—Cl or acid anhydride (R¹—C(O))₂O.

15. A process for the manufacture of a compound of formula (VI) according to claim 1 wherein R is R²—SO₂—; comprising the step of treating a compound of formula (VI^{A}) with a sulfonyl chloride R²—SO₂—Cl.

16. A process for the manufacture of 3-aminomethyl-5-methylhexanoic acid (II) 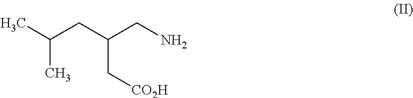
or a pharmaceutically acceptable salt thereof, comprising the steps:
(a) preparing 5-hydroxy-4-(2-methyl-1-propenyl)-5H-2-furanone (VI^{A}) 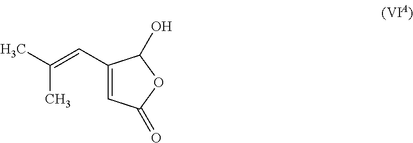
(b) converting said 5-hydroxy-4-(2-methyl-1-propenyl)-5H-2-furanone into 5-hydroxy-4-(2-methylpropyl)-3,4-dihydro-5H-2-furanone (I^{A}) 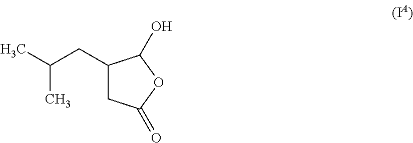
and
(c) converting said 5-hydroxy-4-(2-methylpropyl)-3,4-dihydro-5H-2-furanone into 3-aminomethyl-5-methylhexanoic acid (II).

17. The process according to claim 16 wherein the 3-aminomethyl-5-methylhexanoic acid (II) is (S)-3-aminomethyl-5-methylhexanoic acid ((S)-II) 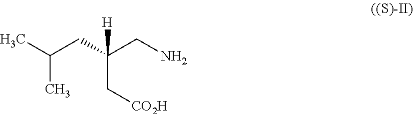
wherein said (S)-3-aminomethyl-5-methylhexanoic acid has an enantiomeric excess of at least 80%.

18. The process according to claim 16 wherein step (a) comprises a process according to claim 11.

19. The process according to claim 16 wherein step (b) comprises the steps:
(b1) treating the 5-hydroxy-4-(2-methyl-1-propenyl)-5H-2-furanone (VI^{A}) with a metal oxide, hydroxide, carbonate or bicarbonate, ammonia, a mono-di- or tri-(C₁-C₃)alkylamine, or a tetra-(C₁-C₃)alkylammonium hydroxide to form a salt of formula (IX) 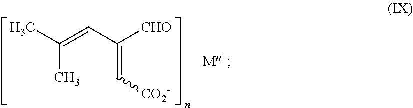
wherein:
n is 1 and M⁺ is selected from Li⁺, Na⁺, K⁺, Rb⁺, NH₄⁺, ((C₁-C₃)alkyl)NH₃⁺, ((C₁-C₃)alkyl)₂NH₂⁺, ((C₁-C₃)alkyl)₃NH⁺ and ((C₁-C₃)alkyl)₄N⁺; or
n is 2 and M²⁺ is selected from Mg²⁺, Ca²⁺ and Zn²⁺;
(b2) hydrogenating the salt of formula (IX) to obtain a salt of formula (X) 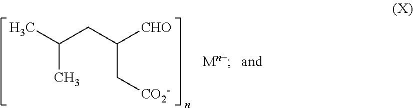
and
(b3) treating the salt of formula (X) with an acid.

20. The process according to claim 16 wherein step (b) comprises the steps:
(b1) converting the 5-hydroxy-4-(2-methyl-1-propenyl)-5H-2-furanone (VI^{A}) to a compound of formula (VI) as defined in claim 3 wherein R is a chiral (C₅-C₁₅)hydrocarbon group;
(b2) hydrogenating the compound of formula (VI) to obtain a compound of formula (XI) 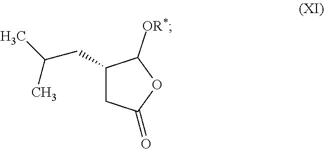
wherein R* is a chiral (C₅-C₁₅)hydrocarbon group; and
(b3) treating the compound of formula (XI) with an acid to give ((S)-I^{A}).

21. A process for the manufacture of 3-aminomethyl-5-methylhexanoic acid (II) 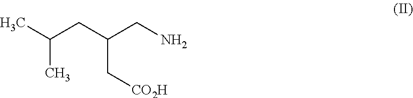
or a pharmaceutically acceptable salt thereof, comprising the steps:
(a) preparing 5-hydroxy-4-(2-methyl-1-propenyl)-5H-2-furanone (VI^{A}) 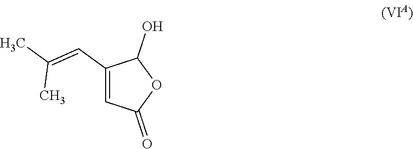
(b) treating the 5-hydroxy-4-(2-methyl-1-propenyl)-5H-2-furanone (VI^{A}) with ammonia or a mono-(C₁-C₃)alkylamine to form a salt of formula (IX^{A}) 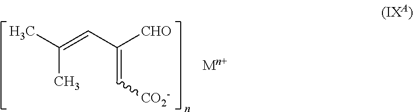
wherein:
n is 1 and M⁺ is selected from NH₄⁺ and ((C₁-C₃)alkyl)NH₃⁺;
(c) hydrogenating the salt of formula (IX^{A}) to obtain a salt of formula (X^{A}) 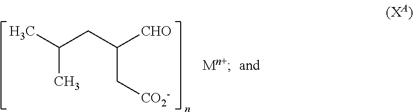
and
(d) treating the salt of formula (X^{A}) with a transaminase or an amine oxidase/imine reductase enzyme to provide 3-aminomethyl-5-methylhexanoic acid (II).

22. The process according to claim 21 wherein the 3-aminomethyl-5-methylhexanoic acid (II) is (S)-3-aminomethyl-5-methylhexanoic acid ((S)-II) 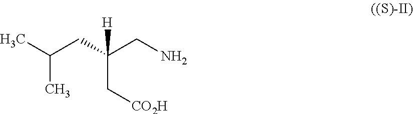
wherein said (S)-3-aminomethyl-5-methylhexanoic acid has an enantiomeric excess of at least 80%.

23. The process according to claim 21 wherein step (a) comprises a process according to claim 11.

24. A process for the manufacture of 5-hydroxy-4-(2-methylpropyl)-3,4-dihydro-5H-2-furanone (I^{A}) 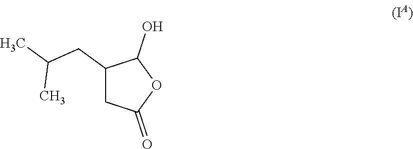
which comprises the steps of:
(a) obtaining 3-isobutylidene-2-oxopentanedioic acid (XII^{A}) or its cyclised isomer (XII^{B}) 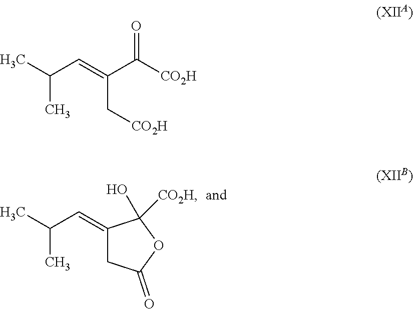
and
(b) sequentially or simultaneously reducing the carbon-carbon double bond and decarboxylating the α-keto acid functional group.

25. The process according to claim 24 wherein the carbon-carbon double bond is reduced to provide 3-isobutyl-2-oxopentanedioic acid (XV) or its cyclised isomer (XV^{A}) 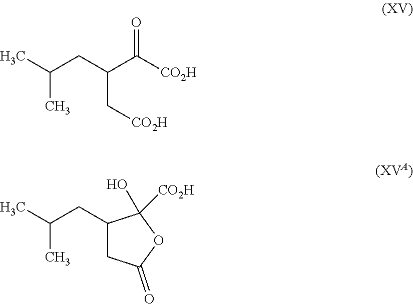
before the decarboxylation of the α-keto acid functional group.

26. The process according to claim 24 wherein the α-keto acid functional group is decarboxylated to provide 3-formyl-5-methyl-3-pentenoic acid (XVI) or its cyclised isomer (XVI^{A}) 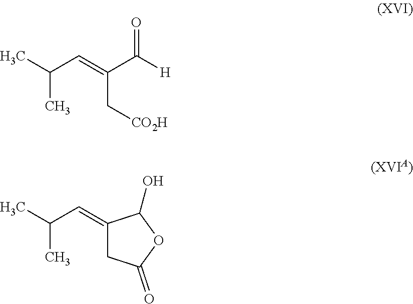
before the reduction of the carbon-carbon double bond.

27. The process according to claim 24 wherein the α-keto acid functional group is decarboxylated and the carbon-carbon double bond is reduced simultaneously.

28. The process according to claim 24 wherein the decarboxylation is carried out in the presence of a decarboxylase enzyme.

29. The process according to claim 24 wherein the reduction of the carbon-carbon double bond is carried out in the presence of an enoate reductase enzyme.

30. A compound selected from the group consisting of:
3-isobutylidene-2-oxopentanedioic acid;
3-isobutyl-2-oxopentanedioic acid; and
3-formyl-5-methyl-3-pentenoic acid,
or a salt, (C₁-C₆)alkyl ester or cyclised isomer thereof.

31. A process for the manufacture of (S)-3-aminomethyl-5-methylhexanoic acid ((S)-II) 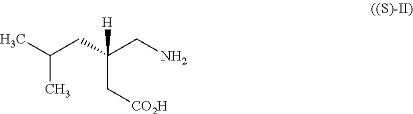
or a pharmaceutically acceptable salt thereof, comprising the steps:
(a) manufacturing 5-hydroxy-4-(2-methylpropyl)-3,4-dihydro-5H-2-furanone (I^{A}) using a process according to any one of claims 23 to 28; and
(b) converting said 5-hydroxy-4-(2-methylpropyl)-3,4-dihydro-5H-2-furanone into (S)-3-aminomethyl-5-methylhexanoic acid.

32. A process for converting (R)-3-aminomethyl-5-methylhexanoic acid into (S)-3-aminomethyl-5-methylhexanoic acid comprising treating the (R)-3-aminomethyl-5-methylhexanoic acid with a transaminase enzyme or an amine dehydrogenase/imine reductase enzyme.

33. A process for increasing the proportion of (S)-3-aminomethyl-5-methylhexanoic acid in a mixture of (R)- and (S)-3-aminomethyl-5-methylhexanoic acid comprising treating the mixture with a transaminase enzyme or an amine dehydrogenase/imine reductase enzyme.

34. A transaminase enzyme having an amino acid sequence that has at least 95% homology to the amino acid sequence
(SEQ ID NO. 1)
MNKPQSWEARAETYSLYGFTDMPSLHX²⁷RGTVVVTHGEGPYX⁴¹VD
VX⁴⁵GRRYLDANSGLYNMVAGFDHKGLIDAAKAQYERFPGYHSFFGRMSD
QTVMLSEKLVEVSPFDSGRVFYTNSGSEANDTMVKMLWFLHAAEGKPQK
RKILTRX¹⁴⁷NAYHGVTAVSASMTGX¹⁶³PX¹⁶⁵NSVFGLPLPGFVHL
X¹⁸⁰CPHYVVRYGEEGETEEQFVARLARELEETIQREGADTIAGFFAEP
VMGAGGVIPPAKGYFQAILPILRKYDIPVISDEVICGFGRTGNIVVGCVT
YDFTPDAIISSKNLTAGFFPVGAVILGPELX³⁰⁴KRLETAIEAIEEFPHG
FTAX³²⁴GHPVGCAIALKAIDVVMNEGLAENVRRLAPRFEERLKHIAE
RPNIGEYRGIGFMWALEAVKDKASKTPFDGNLSVSX⁴⁰¹RIANTC
X⁴⁰⁸DLGLICX⁴¹⁵X⁴¹⁶X⁴¹⁷GQSVILX⁴²⁴PPFILTEAQMDEMF
DKLEKALDKVFAEVA
wherein
X²⁷ is selected from glutamine (Q) and glutamic acid (E);
X⁴¹ is selected from isoleucine (I) and valine (V);
X⁴⁵ is selected from asparigine (N) and histidine (H);
X¹⁴⁷ is selected from asparigine (N) and glutamine (Q);
X¹⁶³ is selected from leucine (L) and methionine (M);
X¹⁶⁵ is selected from tyrosine (Y) and histidine (H);
X¹⁸⁰ is selected from threonine (T); glycine (G) and serine (S);
X³⁰⁴ is selected from alanine (A) and serine (S);
X³²⁴ is selected from glycine (G) and serine (S);
X⁴⁰¹ is selected from lysine (K) and glutamic acid (E);
X⁴⁰⁸ is selected from threonine (T) and glutamine (Q);
X⁴¹⁵ is selected from serine (S) and alanine (A);
X⁴¹⁶ is selected from proline (P) and alanine (A);
X⁴¹⁷ is selected from leucine (L) and methionine (M); and
X⁴²⁴ is selected from cysteine (C) and serine (S).

35. The transaminase enzyme according to claim 34 having the amino acid sequence of SEQ ID NO. 1.

36. The transaminase enzyme according to claim 34,
wherein
X²⁷ is glutamic acid (E);
X¹⁴⁷ is glutamine (Q);
X¹⁶⁵ is histidine (H);
X³⁰⁴ is serine (S);
X³²⁴ is glycine (G);
X⁴⁰¹ is lysine (K);
X⁴⁰⁸ A is glutamine (Q);
X⁴¹⁶ is alanine (A);
X⁴¹⁷ is methionine (M); and
X⁴²⁴ is serine (S).

37. The transaminase enzyme according to claim 35 having an amino acid sequence selected from:
(SEQ ID NO. 2)
MNKPQSWEARAETYSLYGFTDMPSLHQRGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELAKRLETAIEAIEEFPHGFTASGHPVGCAIALKAIDVVMNEGLAENVR
RLAPRFEERLKHIAERPNIGEYRGIGFMWALEAVKDKASKTPFDGNLSVS
ERIANTCTDLGLICSPMGQSVILCPPFILTEAQMDEMFDKLEKALDKVFA
EVA;
(SEQ ID NO. 3)
MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRNNAY
HGVTAVSASMTGLPYNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICSALGQSVILCPPFILTEAQMDEMFDKLEKALDKVFAE
VA;
(SEQ ID NO. 4)
MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICSALGQSVILSPPFILTEAQMDEMFDKLEKALDKVFAE
VA;
(SEQ ID NO. 5)
MNKPQSWEARAETYSLYGFTDMPSLHERGTVVVTHGEGPYIVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGMPHNSVFGLPLPGFVHLTCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PALSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICSAMGQSVILSPPFILTEAQMDEMFDKLEKALDKVFAE
VA;
(SEQ ID NO. 6)
MNKPQSWEARAETYSLYGFTDMPSLHERGTVVVTHGEGPYVVDVNGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLGCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICAAMGQSVILSPPFILTEAQMDEMFDKLEKALDKVFAE
VA;
and
(SEQ ID NO. 7)
MNKPQSWEARAETYSLYGFTDMPSLHERGTVVVTHGEGPYIVDVHGRRYL
DANSGLYNMVAGFDHKGLIDAAKAQYERFPGYHSFFGRMSDQTVMLSEKL
VEVSPFDSGRVFYTNSGSEANDTMVKMLWFLHAAEGKPQKRKILTRQNAY
HGVTAVSASMTGLPHNSVFGLPLPGFVHLSCPHYWRYGEEGETEEQFVAR
LARELEETIQREGADTIAGFFAEPVMGAGGVIPPAKGYFQAILPILRKYD
IPVISDEVICGFGRTGNTWGCVTYDFTPDAIISSKNLTAGFFPVGAVILG
PELSKRLETAIEAIEEFPHGFTAGGHPVGCAIALKAIDWMNEGLAENVRR
LAPRFEERLKHIAERPNIGEYRGIGFMWALEAVKDKASKTPFDGNLSVSK
RIANTCQDLGLICSAMGQSVILSPPFILTEAQMDEMFDKLEKALDKVFAE
VA.

38. A process for the manufacture of (S)-3-aminomethyl-5-methylhexanoic acid ((S)-II) 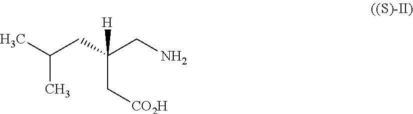
or a pharmaceutically acceptable salt thereof, comprising the step of treating 5-hydroxy-4-(2-methylpropyl)-3,4-dihydro-5H-2-furanone (I^{A}) and an amine with a transaminase enzyme according to claim 34.


SEQUENCE LISTING (15 sequences)

SEQ ID NO: 1 - protein, 453 aa, Artificial Sequence
  Modification of Vibrio fluvialis enzyme
  MISC_FEATURE (27)..(27): Xaa(27) is selected from glutamine (Gln) and glutamic acid (Glu)
  MISC_FEATURE (41)..(41): Xaa(41) is selected from isoleucine (Ile) and valine (Val)
  MISC_FEATURE (45)..(45): Xaa(45) is selected from asparigine (Asn) and histidine (His)
  MISC_FEATURE (147)..(147): Xaa(147) is selected from asparigine (Asn) and glutamine (Gln)
  MISC_FEATURE (163)..(163): Xaa(163) is selected from leucine (Leu) and methionine (Met)
  MISC_FEATURE (165)..(165): Xaa(165) is selected from tyrosine (Tyr) and histidine (His)
  MISC_FEATURE (180)..(180): Xaa(180) is selected from threonine (Thr); glycine (Gly) and serine (Ser)
  MISC_FEATURE (304)..(304): Xaa(304) is selected from alanine (Ala) and serine (Ser)
  MISC_FEATURE (324)..(324): Xaa(324) is selected from glycine (Gly) and serine (Ser)
  MISC_FEATURE (401)..(401): Xaa(401) is selected from lysine (Lys) and glutamic acid (Glu)
  MISC_FEATURE (408)..(408): Xaa(408) is selected from threonine (Thr) and glutamine (Gln)
  MISC_FEATURE (415)..(415): Xaa(415) is selected from serine (Ser) and alanine (Ala)
  MISC_FEATURE (416)..(416): Xaa(416) is selected from proline (Pro) and alanine (Ala)
  MISC_FEATURE (417)..(417): Xaa(417) is selected from leucine (Leu) and methionine (Met)
  MISC_FEATURE (424)..(424): Xaa(424) is selected from cysteine (Cys) and serine (Ser)
MNKPQSWEARAETYSLYGFTDMPSLHXRGTVVVTHGEGPYXVDVXGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRXNAYHGVTAVSASMTGXPXNSVFGLPLPGFVHLXCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELXKRLETAIEAIEEFPHGFTAXGHPVGCAIALKAIDVVMNEGLAENVRRLAPRFEERLKHIAERPNIGEYRGIGFMWALEAVKDKASKTPFDGNLSVSXRIANTCXDLGLICXXXGQSVILXPPFILTEAQMDEMFDKLEKALDKVFAEVA

SEQ ID NO: 2 - protein, 453 aa, Artificial Sequence
  Modification of Vibrio fluvialis enzyme
MNKPQSWEARAETYSLYGFTDMPSLHQRGTVVVTHGEGPYIVDVNGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRNNAYHGVTAVSASMTGLPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELAKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICSPMGQSVILCPPFILTEAQMDEMFDKLEKALDKVFAEVA

SEQ ID NO: 3 - protein, 453 aa, Artificial Sequence
  Modification of Vibrio fluvialis enzyme
MNKPQSWEARAETYSLYGFTDMPSLHERGTVVVTHGEGPYIVDVNGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRNNAYHGVTAVSASMTGLPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELSKRLETAIEAIEEFPHGFTAGGHPVGCAIALKAIDVVMNEGLAENVRRLAPRFEERLKHIAERPNIGEYRGIGFMWALEAVKDKASKTPFDGNLSVSKRIANTCQDLGLICSALGQSVILCPPFILTEAQMDEMFDKLEKALDKVFAEVA

SEQ ID NO: 4 - protein, 453 aa, Artificial Sequence
  Modification of Vibrio fluvialis enzyme
MNKPQSWEARAETYSLYGFTDMPSLHERGTVVVTHGEGPYVVDVNGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRQNAYHGVTAVSASMTGLPHNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELSKRLETAIEAIEEFPHGFTAGGHPVGCAIALKAIDVVMNEGLAENVRRLAPRFEERLKHIAERPNIGEYRGIGFMWALEAVKDKASKTPFDGNLSVSKRIANTCQDLGLICSALGQSVILSPPFILTEAQMDEMFDKLEKALDKVFAEVA

SEQ ID NO: 5 - protein, 453 aa, Artificial Sequence
  Modification of Vibrio fluvialis enzyme
MNKPQSWEARAETYSLYGFTDMPSLHERGTVVVTHGEGPYIVDVNGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRQNAYHGVTAVSASMTGMPHNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPALSKRLETAIEAIEEFPHGFTAGGHPVGCAIALKAIDVVMNEGLAENVRRLAPRFEERLKHIAERPNIGEYRGIGFMWALEAVKDKASKTPFDGNLSVSKRIANTCQDLGLICSAMGQSVILSPPFILTEAQMDEMFDKLEKALDKVFAEVA

SEQ ID NO: 6 - protein, 453 aa, Artificial Sequence
  Modification of Vibrio fluvialis enzyme
MNKPQSWEARAETYSLYGFTDMPSLHERGTVVVTHGEGPYVVDVNGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRQNAYHGVTAVSASMTGLPHNSVFGLPLPGFVHLGCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELSKRLETAIEAIEEFPHGFTAGGHPVGCAIALKAIDVVMNEGLAENVRRLAPRFEERLKHIAERPNIGEYRGIGFMWALEAVKDKASKTPFDGNLSVSKRIANTCQDLGLICAAMGQSVILSPPFILTEAQMDEMFDKLEKALDKVFAEVA

SEQ ID NO: 7 - protein, 453 aa, Artificial Sequence
  Modification of Vibrio fluvialis enzyme
MNKPQSWEARAETYSLYGFTDMPSLHERGTVVVTHGEGPYIVDVHGRRYLDANSGLYNMVAGFDHKGLIDAAKAQYERFPGYHSFFGRMSDQTVMLSEKLVEVSPFDSGRVFYTNSGSEANDTMVKMLWFLHAAEGKPQKRKILTRQNAYHGVTAVSASMTGLPHNSVFGLPLPGFVHLSCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPVGAVILGPELSKRLETAIEAIEEFPHGFTAGGHPVGCAIALKAIDVVMNEGLAENVRRLAPRFEERLKHIAERPNIGEYRGIGFMWALEAVKDKASKTPFDGNLSVSKRIANTCQDLGLICSAMGQSVILSPPFILTEAQMDEMFDKLEKALDKVFAEVA

SEQ ID NO: 8 - protein, 376 aa, Lycopersicon esculentum
MENKVVEEKQVDKIPLMSPCKMGKFELCHRVVLAPLTRQRSYGYIPQPHAILHYSQRSTNGGLLIGEATVISETGIGYKDVPGIWTKEQVEAWKPIVDAVHAKGGIFFCQIWHVGRVSNKDFQPNGEDPISCTDRGLTPQIRSNGIDIAHFTRPRRLTTDEIPQIVNEFRVAARNAIEAGFDGVEIHGAHGYLIDQFMKDQVNDRSDKYGGSLENRCRFALEIVEAVANEIGSDRVGIRISPFAHYNEAGDTNPTALGLYMVESLNKYDLAYCHVVEPRMKTAWEKIECTESLVPMRKAYKGTFIVAGGYDREDGNRALIEDRADLVAYGRLFISNPDLPKRFELNAPLNKYNRDTFYTSDPIVGYTDYPFLETMT

SEQ ID NO: 9 - DNA, 1134 bp, Lycopersicon esculentum
atggaaaacaaagttgtggaagaaaaacaggttgataaaatcccgctgatgagcccgtgtaaaatgggtaaattcgagctgtgtcatcgcgttgtactggcaccgctgactcgtcagcgttcttatggttacattccgcagccgcacgcaatcctgcattactctcagcgcagcaccaacggtggcctgctgatcggtgaagcaaccgtgatcagcgaaactggcatcggttacaaagatgtgccgggtatctggacgaaagagcaggttgaggcctggaaaccgatcgtcgacgcggtgcatgccaaaggtggtattttcttttgtcagatctggcacgttggtcgtgtatccaacaaagattttcagccgaacggcgaagatccgatttcctgtactgaccgcggcctgaccccgcagatccgttccaacggcattgacattgcccacttcacccgtccacgtcgcctgactactgacgagattccgcagatcgtgaacgagttccgcgttgcagcgcgtaatgctattgaagcgggtttcgatggcgtcgagattcatggtgcccacggttacctgatcgaccaattcatgaaagaccaagttaacgaccgcagcgataagtatggcggttctctggagaaccgttgtcgcttcgcgctggaaatcgttgaagcagtagccaacgagattggctccgaccgtgttggtatccgtatctctccattcgcacactacaacgaagcgggcgacactaacccgaccgcactgggcctgtatatggtggagagcctgaataaatacgacctggcgtattgtcacgtggtcgagccgcgcatgaaaaccgcctgggaaaagattgagtgcaccgaaagcctggtgccgatgcgtaaagcctacaaaggcaccttcatcgtagctggtggctacgaccgtgaagacggtaaccgcgctctgatcgaagaccgtgccgacctggttgcgtacggtcgtctgttcatcagcaacccagacctgccgaagcgttttgaactgaacgctccgctgaacaaatacaaccgtgacactttctacacttccgacccgatcgttggttacaccgattacccgtttctggaaactatgacttaataa

SEQ ID NO: 10 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcaccagcgtggtaccgtggttgtcacccacggcgagggcccatacatcgtggacgtcaacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtaacaacgcataccacggtgtaactgctgtttccgcttccatgacgggtctgccgtacaactctgtattcggcctgccgctgccgggtttcgttcacctgacctgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggaactggcgaaacgcctggaaacggcgatcgaagctatcgaagagttcccgcacggctttacggccagcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcgagcgtatcgctaacacctgtaccgacctgggcctgatctgtagcccgatgggtcagtccgttatcctgtgcccgccgttcatcctgaccgaggcgcaaatggatgagatgtttgacaaactggagaaggctctggacaaagtctttgcggaggtggcgtaa

SEQ ID NO: 11 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcacgagcgtggtaccgtggttgtcacccacggcgagggcccatacatcgtggacgtcaacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtaacaacgcataccacggtgtaactgctgtttccgcttccatgacgggtctgccgtacaactctgtattcggcctgccgctgccgggtttcgttcacctgacctgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggaactgagcaaacgcctggaaacggcgatcgaagctatcgaagagttcccgcacggctttacggccggcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcaaacgtatcgctaacacctgtcaggacctgggcctgatctgtagcgcgctgggtcagtccgttatcctgtgcccgccgttcatcctgaccgaggcgcaaatggatgagatgtttgacaaactggagaaggctctggacaaagtctttgcggaggtggcgtaa

SEQ ID NO: 12 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcacgagcgtggtaccgtggttgtcacccacggcgagggcccatacgtggtggacgtcaacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtcaaaacgcataccacggtgtaactgctgtttccgcttccatgacgggtctgccgcacaactctgtattcggcctgccgctgccgggtttcgttcacctgacctgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggaactgagcaaacgcctggaaacggcgatcgaagctatcgaagagttcccgcacggctttacggccggcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcaaacgtatcgctaacacctgtcaggacctgggcctgatctgtagcgcgctgggtcagtccgttatcctgagcccgccgttcatcctgaccgaggcgcaaatggatgagatgtttgacaaactggagaaggctctggacaaagtctttgcggaggtggcgtaa

SEQ ID NO: 13 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcacgagcgtggtaccgtggttgtcacccacggcgagggcccatacatcgtggacgtcaacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtcaaaacgcataccacggtgtaactgctgtttccgcttccatgacgggtatgccgcacaactctgtattcggcctgccgctgccgggtttcgttcacctgacctgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggcactgagcaaacgcctggaaacggcgatcgaagctatcgaagagttcccgcacggctttacggccggcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcaaacgtatcgctaacacctgtcaggacctgggcctgatctgtagcgcgatgggtcagtccgttatcctgagcccgccgttcatcctgaccgaggcgcaaatggatgagatgtttgacaaactggagaaggctctggacaaagtctttgcggaggtggcgtaa

SEQ ID NO: 14 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcacgagcgtggtaccgtggttgtcacccacggcgagggcccatacgtggtggacgtcaacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtcaaaacgcataccacggtgtaactgctgtttccgcttccatgacgggtctgccgcacaactctgtattcggcctgccgctgccgggtttcgttcacctgggttgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggaactgagcaaacgcctggaaacggcgatcgaagctatcgaagagttcccgcacggctttacggccggcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcaaacgtatcgctaacacctgtcaggacctgggcctgatctgtagcgcgatgggtcagtccgttatcctgagcccgccgttcatcctgaccgaggcgcaaatggatgagatgtttgacaaactggagaaggctctggacaaagtctttgcggaggtggcgtaa

SEQ ID NO: 15 - DNA, 1362 bp, Artificial Sequence
  Modification of Vibrio fluvialis enzyme
atgaataaaccacaaagctgggaagcgcgtgctgaaacttactctctgtacggcttcactgatatgccatctctgcacgagcgtggtaccgtggttgtcacccacggcgagggcccatacatcgtggacgtccacggtcgccgttacctggacgcaaactccggcctgtacaatatggttgccggcttcgaccacaagggtctgatcgacgcagcaaaggcccagtacgaacgcttcccgggttaccatagcttcttcggtcgtatgtctgatcaaactgttatgctgagcgagaaactggtagaggtgtctccattcgacagcggtcgcgtgttctatactaactccggctccgaggctaacgatactatggtgaaaatgctgtggtttctgcacgccgcagagggcaagccgcaaaaacgcaaaatcctgactcgtcaaaacgcataccacggtgtaactgctgtttccgcttccatgacgggtctgccgcacaactctgtattcggcctgccgctgccgggtttcgttcacctgagctgtccgcactattggcgttacggcgaagaaggtgaaaccgaagagcagtttgttgctcgtctggcccgcgagctggaggaaactatccaacgtgaaggcgcggacacgattgcgggcttctttgctgagccggtcatgggcgcgggcggcgtaatcccgccggcgaaaggttacttccaggcgatcctgccgattctgcgtaagtacgacatcccggttatctctgatgaagttatctgcggctttggtcgtaccggtaatacttggggttgcgttacctatgacttcaccccggatgcgatcatctccagcaaaaatctgaccgccggtttctttccggttggtgctgtgattctgggtccggaactgagcaaacgcctggaaacggcgatcgaagctatcgaagagttcccgcacggctttacggccggcggtcacccggtgggttgcgctatcgctctgaaagcaatcgatgttgtgatgaatgagggtctggcagagaacgtgcgccgcctggcaccgcgttttgaggagcgtctgaaacacattgccgaacgtccgaacatcggtgaatatcgtggcatcggttttatgtgggcactggaggctgtgaaagacaaagcatctaaaaccccattcgatggtaatctgtctgtgagcaaacgtatcgctaacacctgtcaggacctgggcctgatctgtagcgcgatgggtcagtccgttatcctgagcccgccgttcatcctgaccgaggcgcaaatggatgagatgtttgacaaactggagaaggctctggacaaagtctttgcggaggtggcgtaa